SEQUENCE LISTING (136 sequences)

SEQ ID NO: 1 - DNA, 20 bp, Homo sapiens
ggagccatggattgcacatt

SEQ ID NO: 2 - DNA, 18 bp, Homo sapiens
ggcccgggaagtcactgt

SEQ ID NO: 3 - DNA, 22 bp, Homo sapiens
agccgtgaccactgacaacgag

SEQ ID NO: 4 - DNA, 23 bp, Homo sapiens
gctgcatggttctgagtgctaag

SEQ ID NO: 5 - DNA, 19 bp, Homo sapiens
ccacagctgctgcagaaca

SEQ ID NO: 6 - DNA, 18 bp, Homo sapiens
gaagggtcgcatggcaaa

SEQ ID NO: 7 - DNA, 18 bp, Homo sapiens
tgggcaaaatggcaagga

SEQ ID NO: 8 - DNA, 21 bp, Homo sapiens
tctgccccaggaatcaaaaat

SEQ ID NO: 9 - DNA, 21 bp, Homo sapiens
catgatggagaacgccatcag

SEQ ID NO: 10 - DNA, 21 bp, Homo sapiens
caccgtgaggatgatgttggg

SEQ ID NO: 11 - DNA, 20 bp, Homo sapiens
tttgggcatcagtggaggtc

SEQ ID NO: 12 - DNA, 20 bp, Homo sapiens
cctggaggttgggattgctt

SEQ ID NO: 13 - DNA, 20 bp, Homo sapiens
cacagtcagacttccagctc

SEQ ID NO: 14 - DNA, 20 bp, Homo sapiens
atgggtttggtctcaagtgg

SEQ ID NO: 15 - DNA, 20 bp, Homo sapiens
ccctcgagtcagtacggatg

SEQ ID NO: 16 - DNA, 21 bp, Homo sapiens
gcattccttcgggaatacttt

SEQ ID NO: 17 - DNA, 20 bp, Homo sapiens
cctcctcttcttcatcaggg

SEQ ID NO: 18 - DNA, 20 bp, Homo sapiens
gagtgactcggactcggaag

SEQ ID NO: 19 - DNA, 20 bp, Homo sapiens
gatcatcggttttgggatgt

SEQ ID NO: 20 - DNA, 20 bp, Homo sapiens
ataaaccggttcacaaggcg

SEQ ID NO: 21 - DNA, 20 bp, Homo sapiens
attctgagaaggggtctccc

SEQ ID NO: 22 - DNA, 18 bp, Homo sapiens
aagaagggcagcgagcag

SEQ ID NO: 23 - DNA, 20 bp, Homo sapiens
tcctcaagcccagcattact

SEQ ID NO: 24 - DNA, 19 bp, Homo sapiens
caagaaaggcagcgaagtg

SEQ ID NO: 25 - DNA, 22 bp, Homo sapiens
attggcgatgacctcagtgtga

SEQ ID NO: 26 - DNA, 22 bp, Homo sapiens
gcaatagttggctatgatgccg

SEQ ID NO: 27 - DNA, 20 bp, Homo sapiens
gtatggaatcctgtggcatc

SEQ ID NO: 28 - DNA, 20 bp, Homo sapiens
aagcacttgcggtgcacgat

SEQ ID NO: 29 - DNA, 23 bp, Homo sapiens
gatatctgtcaggagcagtttct

SEQ ID NO: 30 - DNA, 21 bp, Homo sapiens
ctcctcgtcctctcacagata

SEQ ID NO: 31 - DNA, 24 bp, Homo sapiens
aacagcagcagagacaaatgcacc

SEQ ID NO: 32 - DNA, 24 bp, Homo sapiens
tgcagttccagagagttccacact

SEQ ID NO: 33 - DNA, 24 bp, Homo sapiens
aaggaggatgccctgaacctgaaa

SEQ ID NO: 34 - DNA, 24 bp, Homo sapiens
tgcaccttatggatgggaaaggga

SEQ ID NO: 35 - DNA, 24 bp, Homo sapiens
tgaatggctgcagtgacccagata

SEQ ID NO: 36 - DNA, 24 bp, Homo sapiens
tggatgtggagccagtggaagaat

SEQ ID NO: 37 - DNA, 22 bp, Homo sapiens
ctctgcccaatgttaaccagat

SEQ ID NO: 38 - DNA, 20 bp, Homo sapiens
gagtgctccgagatgaatcc

SEQ ID NO: 39 - DNA, 22 bp, Homo sapiens
aggatgcagaaggagatcactg

SEQ ID NO: 40 - DNA, 24 bp, Homo sapiens
gggtgtaacgcaactaagtcatag

SEQ ID NO: 41 - DNA, 22 bp, Homo sapiens
gcgagtgctgatacctgtcaag

SEQ ID NO: 42 - DNA, 20 bp, Homo sapiens
tcattgagccctttcccaat

SEQ ID NO: 43 - DNA, 18 bp, Homo sapiens
ggccgagatgtgcgaact

SEQ ID NO: 44 - DNA, 22 bp, Homo sapiens
ttgttgatgagctggagcatgt

SEQ ID NO: 45 - DNA, 19 bp, Homo sapiens
gcgttctggagaccatgga

SEQ ID NO: 46 - DNA, 25 bp, Homo sapiens
acaaagttgctctgaaaacaaatca

SEQ ID NO: 47 - DNA, 22 bp, Homo sapiens
atttgctcaccgtggagatgtt

SEQ ID NO: 48 - DNA, 25 bp, Homo sapiens
gaagtcatccaggccactactaatg

SEQ ID NO: 49 - DNA, 22 bp, Homo sapiens
tcacagtgactgagcttcagca

SEQ ID NO: 50 - DNA, 22 bp, Homo sapiens
tcatcttcatcacacccaggac

SEQ ID NO: 51 - DNA, 25 bp, Homo sapiens
ccctcaatgaatgtactgaaggatt

SEQ ID NO: 52 - DNA, 23 bp, Homo sapiens
tgtgaagtgaagcagaccaatgt

SEQ ID NO: 53 - DNA, 18 bp, Homo sapiens
ccgggcagagctcaggat

SEQ ID NO: 54 - DNA, 23 bp, Homo sapiens
gaagcagaccaatgtttcaatgg

SEQ ID NO: 55 - DNA, 20 bp, Homo sapiens
ggatagggttggagtcagca

SEQ ID NO: 56 - DNA, 20 bp, Homo sapiens
cttgccgcttcagtttcttc

SEQ ID NO: 57 - DNA, 22 bp, Homo sapiens
atggacaccaaacatttcctgc

SEQ ID NO: 58 - DNA, 19 bp, Homo sapiens
ccagtggagagccgattcc

SEQ ID NO: 59 - DNA, 21 bp, Homo sapiens
agcttgcccgaattgtatgtg

SEQ ID NO: 60 - DNA, 23 bp, Homo sapiens
tctgttgtgaaccatgtgacttc

SEQ ID NO: 61 - DNA, 18 bp, Homo sapiens
gccgtgaactgggtcgaa

SEQ ID NO: 62 - DNA, 25 bp, Homo sapiens
gcatatatagcaatgtctcctgcaa

SEQ ID NO: 63 - DNA, 22 bp, Homo sapiens
caggaagtaagatgcctggaac

SEQ ID NO: 64 - DNA, 21 bp, Homo sapiens
ttcacccccttggatgacatt

SEQ ID NO: 65 - DNA, 20 bp, Homo sapiens
gacttggtgcaatggactcg

SEQ ID NO: 66 - DNA, 21 bp, Homo sapiens
ggtcttgcttgtagaatctgg

SEQ ID NO: 67 - DNA, 21 bp, Homo sapiens
aggctggatctcaaggacaat

SEQ ID NO: 68 - DNA, 19 bp, Homo sapiens
gccagactagcatggcctg

SEQ ID NO: 69 - DNA, 21 bp, Homo sapiens
ttcccggacctgtttctggat

SEQ ID NO: 70 - DNA, 19 bp, Homo sapiens
ctctgggtgcgagtgaagg

SEQ ID NO: 71 - DNA, 22 bp, Homo sapiens
catcggtattaagcaggtgtgg

SEQ ID NO: 72 - DNA, 21 bp, Homo sapiens
gtaccgagacatcaccttgct

SEQ ID NO: 73 - DNA, 19 bp, Homo sapiens
gtagtggcctcggagcaga

SEQ ID NO: 74 - DNA, 22 bp, Homo sapiens
gtggttctcaatgacccagtca

SEQ ID NO: 75 - DNA, 22 bp, Homo sapiens
gtcagtgaacaacttccccact

SEQ ID NO: 76 - DNA, 21 bp, Homo sapiens
acttcggagaggtctccctca

SEQ ID NO: 77 - DNA, 21 bp, Homo sapiens
cacagttacctgagcacctga

SEQ ID NO: 78 - DNA, 20 bp, Homo sapiens
accagtaagagggtgcctca

SEQ ID NO: 79 - DNA, 21 bp, Homo sapiens
ccttggccatgcaatagcatt

SEQ ID NO: 80 - DNA, 22 bp, Homo sapiens
gaaggtaagtgaaggtcttgcc

SEQ ID NO: 81 - DNA, 20 bp, Homo sapiens
tccctgacgtcctcacctac

SEQ ID NO: 82 - DNA, 20 bp, Homo sapiens
ccccttccgaatcttcacta

SEQ ID NO: 83 - DNA, 23 bp, Homo sapiens
accagccatcttgatgaaaaaca

SEQ ID NO: 84 - DNA, 23 bp, Homo sapiens
cagcaactattggtgaaacaacc

SEQ ID NO: 85 - DNA, 21 bp, Homo sapiens
tcgtgggggaccctataaaac

SEQ ID NO: 86 - DNA, 23 bp, Homo sapiens
cgtcctccgcttgataataagtc

SEQ ID NO: 87 - DNA, 20 bp, Homo sapiens
gcactgacatccaggacaac

SEQ ID NO: 88 - DNA, 20 bp, Homo sapiens
agccactttttctgggtcct

SEQ ID NO: 89 - DNA, 24 bp, Homo sapiens
accatacgtttgctggaaaccatc

SEQ ID NO: 90 - DNA, 24 bp, Homo sapiens
agcgcccgtataaaaaggaaccaa

SEQ ID NO: 91 - DNA, 22 bp, Homo sapiens
tctgtattccagtgtgatgtgc

SEQ ID NO: 92 - DNA, 20 bp, Homo sapiens
cttttggcccgtgacgtaat

SEQ ID NO: 93 - DNA, 20 bp, Homo sapiens
cgcggatctgatgcgtcgct

SEQ ID NO: 94 - DNA, 20 bp, Homo sapiens
cggccctggcagttctgtgg

SEQ ID NO: 95 - DNA, 18 bp, Homo sapiens
gctgggtgcagacgcttt

SEQ ID NO: 96 - DNA, 22 bp, Homo sapiens
tgccgtcagttcttgtgtgact

SEQ ID NO: 97 - DNA, 21 bp, Homo sapiens
gagacccagaggctacagatt

SEQ ID NO: 98 - DNA, 21 bp, Homo sapiens
aatgtactccacatggggcaa

SEQ ID NO: 99 - DNA, 21 bp, Homo sapiens
aaacagggaagatcgagccag

SEQ ID NO: 100 - DNA, 22 bp, Homo sapiens
ggtctgaccaagctatcaggtt

SEQ ID NO: 101 - DNA, 19 bp, Homo sapiens
cgtgggctccagcattcta

SEQ ID NO: 102 - DNA, 22 bp, Homo sapiens
tcaccagtcatttctgcctttg

SEQ ID NO: 103 - DNA, 21 bp, Homo sapiens
cagtgccctgagcacagtcaa

SEQ ID NO: 104 - DNA, 22 bp, Homo sapiens
gctgctcccgcaataaagtacc

SEQ ID NO: 105 - DNA, 21 bp, Homo sapiens
gcttgttggcctcagttaagg

SEQ ID NO: 106 - DNA, 21 bp, Homo sapiens
gtagctcaggcgtacagagat

SEQ ID NO: 107 - DNA, 22 bp, Homo sapiens
ctttcctactctgtacccgagg

SEQ ID NO: 108 - DNA, 21 bp, Homo sapiens
cggggcattccattgataagg

SEQ ID NO: 109 - DNA, 20 bp, Homo sapiens
tatgtggatgcggtcaaaga

SEQ ID NO: 110 - DNA, 20 bp, Homo sapiens
acggttgaacccagagtgtc

SEQ ID NO: 111 - DNA, 18 bp, Homo sapiens
acgggaaggactgcagca

SEQ ID NO: 112 - DNA, 21 bp, Homo sapiens
gcagcttcatgatggcagact

SEQ ID NO: 113 - DNA, 20 bp, Homo sapiens
gcccaaggctgaactcagta

SEQ ID NO: 114 - DNA, 20 bp, Homo sapiens
agcttggcttctagccgatt

SEQ ID NO: 115 - DNA, 21 bp, Homo sapiens
gggctgccttggagtgacgtc

SEQ ID NO: 116 - DNA, 22 bp, Homo sapiens
agtccccagtcacatgacaaag

SEQ ID NO: 117 - DNA, 25 bp, Homo sapiens
ggagggcagcctctagcactgtcaa

SEQ ID NO: 118 - DNA, 25 bp, Homo sapiens
tcagtctgtaggtcaatccagccct

SEQ ID NO: 119 - DNA, 21 bp, Homo sapiens
ggcctcccaacattcattaac

SEQ ID NO: 120 - DNA, 22 bp, Homo sapiens
gtagcccgccctccttgcttta

SEQ ID NO: 121 - DNA, 21 bp, Homo sapiens
ccaaggcctcctcatctgatg

SEQ ID NO: 122 - DNA, 21 bp, Homo sapiens
gtctacacacaaagccccgag

SEQ ID NO: 123 - DNA, 21 bp, Homo sapiens
ctgcccggcttcttgagtaag

SEQ ID NO: 124 - DNA, 21 bp, Homo sapiens
tcccctacttgtcggctctac

SEQ ID NO: 125 - DNA, 21 bp, Homo sapiens
tagcaccaagctccctccaag

SEQ ID NO: 126 - DNA, 21 bp, Homo sapiens
tcctgttcccaacgtcaccag

SEQ ID NO: 127 - DNA, 22 bp, Homo sapiens
gattcgcagcgcatcgccttct

SEQ ID NO: 128 - DNA, 21 bp, Homo sapiens
taccggtggatgtggaatgtg

SEQ ID NO: 129 - DNA, 21 bp, Homo sapiens
cagggtttctctgtgtagccc

SEQ ID NO: 130 - DNA, 21 bp, Homo sapiens
agtggcgcacgcctttaatcc

SEQ ID NO: 131 - DNA, 24 bp, Homo sapiens
ttgggaaagaggtatggcttggca

SEQ ID NO: 132 - DNA, 24 bp, Homo sapiens
aagaagttcctgcctaactctccc

SEQ ID NO: 133 - DNA, 22 bp, Homo sapiens
tgcctgcattaccggtcgatgc

SEQ ID NO: 134 - DNA, 23 bp, Homo sapiens
ccatgagtgaacgaacctggtcg

SEQ ID NO: 135 - DNA, 20 bp, Homo sapiens
tagaggagctttggttcgtg

SEQ ID NO: 136 - DNA, 20 bp, Homo sapiens
aatagccttcatagccttcg

(19) United States
(12) Patent Application Publication
(10) Pub. No.: US 2022/0119511 A1
(43) Pub. Date: Apr. 21, 2022

(54) COMPOSITIONS AND METHODS FOR TREATMENT OF DIABETES, OBESITY, HYPER-CHOLESTEROLEMIA, AND ATHEROSCLEROSIS BY INHIBITION OF SAM68

(71) Applicant: The UAB Research Foundation, Birmingham, AL (US)

(72) Inventors: Gangjian QIN, Birmingham, AL (US); Aijun QIAO, Birmingham, AL (US)

(73) Assignee: The UAB Research Foundation, Birmingham, AL (US)

(21) Appl. No.: 17/503,113

(22) Filed: Oct. 15, 2021

Related U.S. Application Data
(60) Provisional application No. 63/092,905, filed on Oct. 16, 2020.

(51) Int. Cl. C07K 16/18; A61K 31/713

(52) U.S. Cl. CPC C07K 16/18; A61K 31/713

(57) ABSTRACT

Disclosed are novel treatments for diseases and conditions caused by, directly or indirectly, high blood glucose levels increased gluconeogenesis. Such disease and conditions include, but are not limited to, type II diabetes, obesity, and cardiovascular conditions. Sam68, an RNA-binding adaptor protein and Src kinase substrate, is a novel regulator of hepatic gluconeogenesis and global and hepatic deletions of Sam68 significantly reduce blood glucose levels and the glucagon-induced expression of gluconeogenic genes. The treatments described herein may include inhibition of the activity of Sam68.

### RELATED APPLICATION

This application claims priority to U.S. Provisional Application No. 63/092,905, filed on Oct. 16, 2020 and titled “Methods of Treatment of Diabetes and Obesity by Inhibition of SAM68”, the entire contents of which are incorporated herein by reference.

### BACKGROUND

Hepatic gluconeogenesis is essential for glucose homeostasis and represents a therapeutic target for treatment of type 2 diabetes. However, the mechanism of hepatic gluconeogenesis is incompletely understood. Of the estimated 34.3 million people in the US with diabetes, 90-95% have Type 2 diabetes (T2D), which is frequently accompanied by other comorbid diseases. T2D contributes to the mortality of many chronic health conditions, including cardiovascular disease, stroke, and kidney disease¹.

In healthy individuals, blood glucose levels are stabilized via a balance between glucose consumption in the peripheral tissues and glucose production. Approximately 90% of glucose production occurs in the liver. Although the mechanisms of T2D are complex, hepatic glucose production is considered a first-line therapeutic target². The liver produces glucose via two distinct processes, glycogenolysis (breakdown of the molecule glycogen into glucose) and gluconeogenesis (generation of glucose from certain non-carbohydrate carbon substrates, such as, but not limited to glucogenic amino acids, glycerol, free chain fatty acids, and lactate in humans). Gluconeogenesis appears to be the major cause of elevated glucose production in T2D³. As a result, drugs that downregulate gluconeogenesis, such as metformin, are among the most common treatments for hyperglycemia in patients with T2D⁴⁻⁶.

Both glycogenolysis and gluconeogenesis are governed primarily by glucagon and insulin, which upregulate and downregulate glucose production, respectively^{7,8}. The signaling mechanisms induced by these two counter-regulatory hormones converge on CRTC2, a member of a family of downstream transcriptional coactivators that are regulated by cyclic AMP response element (CRE) binding protein (CREB), and of the three known isoforms of CREB-regulated transcriptional coactivators (also including CRTC1 and CRTC3)⁹. CRTC2 is most abundantly expressed in the liver¹⁰. In fasting animals, glucagon signaling increases CRTC2 levels and dephosphorylates CRTC2¹¹, which then translocates from the cytosol into the nucleus where it binds phosphorylated CREB and activates transcription of the master gluconeogenic regulator PPARγ coactivator 1α (PGC-1α) and two enzymes that catalyze the rate-limiting steps of gluconeogenesis, phosphoenolpyruvate carboxykinase (PEPCK) and glucose-6-phosphatase (G6Pase)¹²⁻¹⁴.

When feeding is re-initiated, the insulin pathway is upregulated, which promotes CRTC2 ubiquitination and degradation, thereby terminating gluconeogenesis¹⁵. Thus, since diabetic hyperglycemia occurs when glucagon levels (or the physiological sensitivity to glucagon) increase and/or insulin levels (or sensitivity) decline^{16,17} strategies that manipulate the CREB/CRTC2 complex may be effective for the treatment of T2D.

Src-associated-in-mitosis-of-68k Da (Sam68; also known as KH-domain-containing, RNA-binding, signal-transduction-associated 1 [KHDRBS1]) is a member of the signal-transducer-and-activator-of-RNA (STAR) family of RNA-binding proteins^{18,19} and participates in numerous cellular functions, including RNA processing^{20,21,} kinase- and growth-factor-signaling^{22,23}, transcription^{24,25}, cell-cycle regulation, and apoptosis^{26,27}. Its range of activity is reflected in the wide variety of phenotypes observed in Sam68 knockout (Sam68^{-/-}) mice, which includes defects in spermatogenesis^{28,29} and adipogenic differentiation³⁰, as well as a relatively lean body mass coupled with increases in adipose thermogenesis and an improved systemic glucose profile when fed a high-fat diet (HFD)³¹.

Currently, diabetes is treated with 6 classes of medications: 1) Metformin inhibits hepatic glucose production and improves the sensitivity of peripheral tissues to insulin. Side effects include nausea and diarrhea. 2) Sulfonylureas, DPP-4 inhibitors and meglitinides promote islet secretion of insulin. Side effect include weight gain. 3) GLP-1 receptor agonists slow food digestion and lower blood sugar levels. Side effects include nausea and increased risk of pancreatitis. 4) SGLT2 inhibitors are the newest diabetes drugs on the market and prevent the kidney from reabsorbing sugar into the blood. Side effects may include yeast infections and urinary tract infections, increased urination and hypotension. 5) Thiazolidinediones enhance peripheral tissues sensitivity to insulin. Side effects include weight gain and other more-serious side effects, such as an increased risk of heart failure and fractures. Because of these risks, these medications generally aren't a first-choice treatment. 6) Insulin promotes uptake of blood glucose by peripheral tissues. Side effects include hypoglycemia, headache, weight gain, rash, itching, flu-like symptoms, lipoatrophy, and pain and reaction at the site of injection. Despite the availability of many treatment possibilities, the significant drawbacks with regard to improving cardiovascular outcome by these medications have limited their effective use for treatment of T2D, obesity and related conditions. The incidence of diabetes is on the rise, and perhaps one of the major factors why the life expectancy in the US are falling. Thus, it is urgent to find alternative therapeutic drugs to improve safe therapy of T2D, obesity, and related conditions and reduce the associated cardiovascular morbidity and mortality. The present disclosure provides such an alternative.

### SUMMARY OF THE DISCLOSURE

The present disclosure provides for novel treatments for type II diabetes, obesity, and related conditions, including cardiovascular conditions of the heart, eye and limbs. The present disclosure demonstrates that Sam68, an RNA-binding adaptor protein and Src kinase substrate, is a novel regulator of hepatic gluconeogenesis. Both global and hepatic deletions of Sam68 significantly reduce blood glucose levels and the glucagon-induced expression of gluconeogenic genes. In addition, protein levels of CRTC2, a crucial transcriptional regulator of gluconeogenesis, are >50% lower in Sam68-deficient hepatocytes than in wild-type hepatocytes; mRNA expression was not inhibited. The present disclosure demonstrates that Sam68 interacts with CRTC2 and reduces CRTC2 ubiquitination. However, truncated mutants of Sam68 that lack the C-(Sam68^{ΔC}) or N-terminal (Sam68ΔN) domains fails to bind CRTC2 or to stabilize CRTC2 protein, respectively, and transgenic Sam68ΔN mice recapitulate the blood-glucose and gluconeogenesis profile of Sam68-deficient mice. Hepatic Sam68 expression is also upregulated in patients with diabetes and in two diabetic mouse models, while hepatocyte-specific Sam68 deficiencies alleviate diabetic hyperglycemia and improves insulin sensitivity in mice. Thus, these disclosed results identify a previously unrecognized role of Sam68 in hepatic gluconeogenesis, and novel therapies targeting Sam68 may normalize glycemia in patients with diabetes.

The present disclosure demonstrates that Sam68 deletions, both globally and when restricted to the liver, reduce blood-glucose levels in mice by impeding gluconeogenesis, and that these effects are at least partially mediated by declines in CRTC2 protein stability and CRTC2/CREB-induced activation of gluconeogenic gene transcription. Inhibition of Sam68 lead to a significant reduction of body fat and blood glucose levels. The results also demonstrate that hepatic Sam68 deficiencies improve insulin sensitivity and reduce hyperglycemia in diabetic mice, which suggests that Sam68 could be a novel therapeutic target for the treatment of T2D, obesity and related conditions, including cardiovascular conditions of the heart, eye and limbs.

In some embodiments, the present disclosure relates to a composition comprising an effective amount of at least one inhibitor of Sam68, or a pharmaceutically acceptable form thereof. In some embodiments, the at least one inhibitor is selected from the group consisting of: small molecule inhibitors, peptide inhibitors, antibodies, viral vectors expressing an inhibitor RNA, and inhibitory RNA. In some embodiments, the at least one inhibitor of Sam68 comprises inhibitor RNA selected from the group consisting of: shRNA, siRNA, microRNA, and antisense-RNA. In some embodiments, the at least one inhibitor RNA comprises a sequence that is specifically hybridisable to a target Sam68 nucleotide sequence. In some embodiments, the at least one inhibitor of Sam68 comprises an antibody configured to bind to a target selected from the group consisting of: the N-terminal domain of Sam68 and the C-terminal domain of Sam68. In some embodiments, the at least one inhibitor of Sam68 comprises an antibody configured to decrease the association of Sam68 with at least one protein. In some embodiments, the at least one protein comprises CRTC2.

In some embodiments, the present disclosures relates to a method for treating a Sam68-mediated condition in a subject, comprising administering to said subject a therapeutically effective amount of at least one inhibitor of Sam68 that inhibits an activity of Sam68, or a pharmaceutically acceptable form thereof. In some embodiments, the at least one inhibitor is selected from the group consisting of: small molecule inhibitors, peptide inhibitors, antibodies, viral vectors ex-pressing an inhibitor RNA, and inhibitory RNA. In some embodiments, the at least one inhibitor of Sam68 comprises inhibitor RNA selected from the group consisting of: shRNA, siRNA, microRNA, and anti-sense-RNA. In some embodiments, the at least one inhibitor RNA comprises a sequence that is specifically hybridisable to a target Sam68 nucleotide sequence. In some embodiments, the at least one inhibitor comprises an antibody con-figured to bind to a target selected from the group consisting of: the N-terminal do-main of Sam68 and the C-terminal domain of Sam68. In some embodiments, the at least one inhibitor comprises an antibody con-figured to decrease the association of Sam68 with another protein. In some embodiments, the Sam68-mediated condition is selected from the group consisting of: type 1 diabetes, type 2 diabetes, obesity, cardiovascular disease, kidney disease, hypercholesterolemia, atherosclerosis and hyperglycemia. In some embodiments, the subject is human. In some embodiments, the subject is diagnosed with or suspected of having at least one condition selected from the group consisting of: type 1 diabetes, type 2 diabetes, obesity, cardiovascular disease, kidney disease, hypercholesterolemia, atherosclerosis and hyperglycemia. In some embodiments, the administration of the at least one inhibitor or pharmaceutically acceptable form thereof comprises two or more doses. In some embodiments, the Sam68-mediated condition is treated or prevented. In some embodiments, the administration is parenteral, pulmonary, intra-nasal, oral-gastric, buccal, or intravenous.

### BRIEF DESCRIPTION OF THE FIGURES

FIGS. 1A-1V show hepatic Sam68 deficiency reduces blood glucose levels. Blood glucose levels were measured in Sam68^{-/-} and WT mice (FIG. 1A) under feeding conditions and after the animals were fasted for 5 h or 16 h, and at the indicated time points after the administration of (FIG. 1B) sodium pyruvate (pyruvate tolerance test [PTT]; WT, n=11; Sam68^{-/-}, n=9) or (FIG. 1C) glucagon (glucagon tolerance test [GcTT]; WT, n=10; Sam68^{-/-}, n=9). Blood glucose levels were measured in Sam68^{LKO} (n=9-13) and Sam68^{f/f} (n=7-12) mice (FIG. 1D) under feeding conditions and after the animals were fasted for 5 h or 16 h, (FIG. 1E) in the PTT and (FIG. 1F) GcTT, and at the indicated time points after the administration of glucose (glucose tolerance test [GTT]) (FIG. 1G) or insulin (insulin-tolerance test [ITT]) (FIG. 1H). In FIG. 1I, protein levels of phosphorylated AKT (at amino acids S473 and T308) and total AKT in the liver of Sam68^{LKO} and Sam68^{f/f} mice were evaluated via Western blot at 20 min after intraperitoneal injection of insulin (1 U/kg) (+) or saline (−). In FIG. 1J, serum insulin levels in Sam68^{LKO} and Sam68^{f/f} mice at feeding condition or after 16 h fasting. Hyperinsulinemic-euglycemic clamping studies were conducted in Sam68^{LKO} (n=7-8) and Sam68^{f/f} (n=7-8) mice. Mice were infused with radiolabeled ([3-³H]) glucose for 120 min; then, insulin infusion was initiated and maintained at a constant rate for 120 min, and (FIG. 1K, left) the rate of [3-³H] glucose infusion was adjusted to maintain euglycemia (100 mg/dL). In FIG. 1K (right), the glucose infusion rate (GIR) was calculated for the last 40 min of insulin infusion. Basal and clamped rates of (FIG. 1L) glucose disposal and (FIG. 1M) glucose production were calculated from the GIR and measurements of the plasma glucose specific activity. In FIG. 1N, glycogen synthesis rate was determined by dividing the hepatic tracer glycogen infusion rate (hepatic glycogen ³H dpm/g liver) with plasma tracer glucose specific activity. Sam68^{f/f} mice were injected with AAV8-TBG-iCre (Sam68^{f/f}; AAV-Cre, n=8) to induce a hepatic-specific Sam68 deletion or with control AAV8-TBG-eGFP (Sam68^{f/f}; AAV-GFP, n=8). Three weeks later, blood glucose levels were measured under (FIG. 1O) feeding conditions or after the animals were fasted for 16 h, and in the (FIG. 1P) PTT, (FIG. 1Q) GcTT, and (FIG. 1R) GTT. FIG. 1S shows body weight of WT mice and Sam68^{KO} mice (n=6-10/group, *p<0.05, **p<0.01). FIG. 1T shows body compositions, fat mass (left panel and lean mass (right panel), assessed by NMR in 6-month-old mice (n=6-10/group, *p<0.05). FIG. 1U shows gross morphology of 6-month-old WT and Sam68^{KO} mice and BAT, inguinal and epididymal fat pads. FIG. 1V shows food intake measured daily for 3 days in 4-month-old mice and averaged (n=6/group, N.S., not significant). Data are expressed as mean±standard error of the mean (s.e.m.). *p<0.05, **p<0.01, ***p<0.001, ****p<0.0001 at the same time point, NS, not significant (FIGS. 1A, 1D, 1J, 1N, 1O, and right panel of 1K: unpaired t test; FIGS. 1B-1O, 1E-1H, 1L-1M, 1P-1R, and left panel of 1K: two-way ANOVA).

FIGS. 2A-2H show generation and metabolic characterization of hepatocyte-specific Sam68 knockout mice. FIG. 2A shows the structural components of the Sam68 gene (upper panel) and protein (lower panel). Sam68 contains a GSG domain composed of a single RNA binding KH domain flanked by NK (N-terminus of KH) and CK (C-terminus of KH) segments; six consensus proline-rich motifs (P0-P5); RGG boxes; a C-terminal tyrosine-rich domain (YY), and a nuclear localization signal (NLS). Amino acid positions are numbered across the bottom of the protein structure. FIG. 2B shows the gene-targeting strategy for floxing exons 5-8 of Sam68 in embryonic stem (ES) cells. FIG. 2C and FIG. 2D show Southern blotting and PCR analyses, respectively, to confirm that the ES clones were correctly targeted. FIG. 2E shows the genotypes of the WT and floxed alleles were confirmed via PCR. In FIG. 2F, Sam68 protein expression was evaluated via Western blot in the liver, spleen, epididymal fat, brown fat, skeletal muscle, subcutaneous fat, brain, heart, lung, and kidney tissues of Sam68^{LKO} and Sam68^{ff} mice. In FIG. 2G, Sam68_{f/f} mice were injected with AAV8-TBG-iCre (Sam68_{f/f};AAV-Cre) to induce a hepatic-specific Sam68 deletion or with control AAV8-TBG-eGFP (Sam68_{f/f};AAV-GFP). Three weeks later, Sam68 protein levels were evaluated via Western blot in the liver, epididymal fat, brown fat, heart, skeletal muscle, and lung tissues. In FIG. 2H, blood glucose levels were measured in Sam68_{f/f};AAV-Cre (n=8) and Sam68_{f/f};AAV-GFP (n=8) mice at the indicated time points during the ITT. Data are expressed as mean±standard error of the mean (s.e.m.). *p<0.05 (h, two-way ANOVA).

FIGS. 3A-3I show hepatic Sam68 deficiency reduces glucagon signaling and gluconeogenic gene expression. mRNA (FIG. 3A) and protein expression (FIG. 3B) of PGC-1α, PEPCK, and G6Pase were evaluated via qRT-PCR and Western blotting, respectively, in the livers of Sam68^{LKO} and Sam68^{f/f} mice under feeding conditions and after the animals had been fasted for 16 h. In FIG. 3C, glucose production was measured in WT and Sam68^{-/-} primary hepatocytes that had been cultured with (Glucagon) or without (Basal) glucagon (100 nM) for 4 h. In FIG. 3D, mRNA expression of gluconeogenic genes was measured in WT and Sam68^{-/-} primary hepatocytes after treatment with glucagon for 0-3 h (n=3). mRNA expression of the glucagon receptor (GCR) was evaluated (FIG. 3E) in WT and Sam68^{-/-} primary hepatocytes after treatment with glucagon for 0-3 h (n=3) and (FIG. 3F) in the livers of WT and Sam68^{-/-} mice under feeding conditions, after fasting the animals for 16 h, and after 16 h of fasting followed by 4 h of refeeding. In FIG. 3G, mRNA expression of PKA subunits was evaluated in the livers of WT and Sam68^{-/-} mice under feeding conditions or after 16 h of fasting. In FIG. 3H, PKA activity was measured in WT and Sam68^{-/-} primary hepatocytes after treatment with PBS (basal), glucagon, forskolin (10 μM), or Bt2-cAMP (100 μM) for 30 min. In FIG. 3I, WT and Sam68^{-/-} primary hepatocytes were treated with glucagon for 0-30 minutes and for 1-4 hours, then, protein levels of phosphorylated PKA substrates was evaluated. Data are expressed as mean±standard error of the mean (s.e.m.). *p<0.05, **p<0.01, ***p<0.001, NS, not significant (two-way ANOVA).

FIGS. 4A-4O show Sam68 deficiency reduces glucagon signaling and glucose production in hepatocytes. In FIG. 4A, WT and Sam68^{-/-} hepatocytes were treated with 200 nM and 400 nM glucagon for 3 h (n=3), and then the mRNA expression of gluconeogenic genes was measured via qRT-PCR. In FIG. 4B, WT and Sam68^{-/-} hepatocytes were treated with glucagon (100 nM) for 3 h and then with Actinomycin D (10 μg/mL) to block new mRNA synthesis; mRNA expression of gluconeogenic genes was measured 0-8 h later (n=3). In FIG. 4C, mRNA expression of PKA subunits was measured in WT and Sam68^{-/-} hepatocytes (n=4). WT and Sam68^{-/-} primary hepatocytes were treated with (FIG. 4D) forskolin (10 μM) or (FIG. 4E) Bt2-cAMP (100 μM) for 0-30 min and 1-4 h; then, protein levels of phosphorylated PKA substrates was evaluated via Western blot. In FIG. 4F, glucose production was measured in WT and Sam68^{-/-} primary hepatocytes after treatment with forskolin for 4 h. In FIG. 4G, mRNA expression of gluconeogenic genes was measured in WT and Sam68^{-/-} hepatocytes after treatment with forskolin for 0-3 h (n=3). In FIG. 4H, glucose production was measured in WT and Sam68^{-/-} primary hepatocytes after treatment with Bt2-cAMP for 4 h. In FIG. 4I, mRNA expression of gluconeogenic genes was measured in WT and Sam68^{-/-} hepatocytes after treatment with Bt2-cAMP for 0-3 h (n=3). WT and Sam68^{-/-} primary hepatocytes were transduced with adenovirus coding for GFP or a GFP-Sam68 fusion protein; 48 h later, (FIG. 4J) transduction efficiency was evaluated by viewing GFP expression under a fluorescence microscope; (FIG. 4K) Sam68 and CRTC2 protein levels were evaluated via Western blotting; (FIG. 4L) glucose production was measured after treatment with glucagon, forskolin, or Bt2-cAMP for 0-3 h; mRNA expression of gluconeogenic genes was measured after treatment with (FIG. 4M) glucagon, (FIG. 4N) forskolin, or (FIG. 4O) Bt2-cAMP for 0-3 h (n=3). Data are expressed as mean±standard error of the mean (s.e.m.). *p<0.05, **p<0.01, ***p<0.001, NS, not significant (FIGS. 4A-4B, 4F-4I, 4I-4O: two-way ANOVA; FIG. 4C: unpaired t test).

FIGS. 5A-5I show Sam68 enhances glucagon signaling by inhibiting CRTC2 degradation. In FIG. 5A, WT and Sam68^{-/-} primary hepatocytes were treated with glucagon (100 nM) for 0-30 min and 1-4 h and protein levels of phosphorylated CREB (p-CREB), total CREB, and CRTC2 were evaluated via Western blot. In FIG. 5B, WT and Sam68^{-/-} hepatocytes were treated with glucagon, forskolin (10 μM), or Bt2-cAMP (100 μM) for 30 min and CRTC2 occupancy of the promoters for PGC-1α, PEPCK, and G6Pase was evaluated via chromatin immunoprecipitation (ChIP) assay. Sam68^{LKO} and Sam68^{f/f} mice were administered adenoviral vectors coding for GFP or a ubiquitination-defective CRTC2^{K628R} mutant (2×10⁹ pfu per mouse). Four days later, blood glucose levels were measured (FIG. 5C) under feeding conditions, (FIG. 5D) after fasting for 16 h, and in the (FIG. 5E) pyruvate-tolerance test (PTT) (Sam68^{f/f};Ad-GFP, n=9. Sam68^{LKO};Ad-GFP, n=9. Sam68^{f/f};Ad-CRTC2^{K628R,} n=10; Sam68^{LKO};Ad-CRTC2^{K628R}, n=11), (FIG. 5F) glucagon-tolerance test (GcTT) (Sam68^{f/f};Ad-GFP, n=9. Sam68^{LKO};Ad-GFP, n=10. Sam68^{f/f};Ad-CRTC2, n=9. Sam68^{LKO};Ad-CRTC2, n=9), and (FIG. 5G) glucose-tolerance test (Sam68^{f/f};Ad-GFP, n=9. Sam68^{LKO};Ad-GFP, n=10. Sam68^{f/f};Ad-CRTC2^{K628R}, n=9. Sam68^{LKO};Ad-CRTC2^{K628R}, n=9); and (FIG. 5H) mRNA and (FIG. 5I) protein levels of gluconeogenic genes in the liver were analyzed under feeding conditions or after fasting for 16 h. Data are expressed as mean±standard error of the mean (s.e.m.); *p<0.05, **p<0.01, ***p<0.001 (two-way ANOVA).

FIGS. 6A-6M show hepatic Sam68 deficiency reduces glucagon signaling and CRTC2 protein stability. WT and Sam68^{-/-} primary hepatocytes were treated with (FIG. 6A) forskolin (10 μM) and (FIG. 6B) Bt2-cAMP (100 μM) for 0-30 min and 1-4 h; then, protein levels of phosphorylated CREB (p-CREB), total CREB, and CRTC2 were evaluated via Western blot. In FIG. 6C, WT and Sam68^{-/-} primary hepatocytes were treated with PBS or glucagon (100 nM) for 30 min; then, protein levels of p-CREB, total CREB, and CRTC2 in nuclear and cytoplasmic extracts were evaluated by immunoblot. mRNA expression of CRTC1, CRTC2, and CRTC3 was evaluated via qRT-PCR in (FIG. 6D) WT and Sam68^{-/-} primary hepatocytes and in (FIG. 6E) liver tissue from WT and Sam68^{-/-} mice under feeding conditions, after 16 h of fasting, and after 16 h of fasting followed by 4 h of refeeding. In FIG. 6F, mRNA expression of CRTC1, CRTC2, and CRTC3 was evaluated in WT and Sam68^{-/-} hepatocytes after treatment with Actinomycin D (10 μg/mL) for 0-8 h to block new mRNA synthesis (n=3). Sam68^{LKO} and Sam68^{f/f} mice were administered Ad-GFP or Ad-CRTC2^{K628R}. Four days later, mice were sacrificed under feeding conditions or after 16 h of fasting, and CRTC2 mRNA expression was evaluated in liver tissues (FIG. 6G). Four days after Ad-GFP or Ad-CRTC2^{K628R} administration, mice were treated with (+) or without (−) insulin (1 mU/g), and protein levels of phosphorylated AKT (at amino acids S473 and T308) and total AKT were evaluated 20 min later (FIG. 6H). WT and Sam68^{-/-} primary hepatocytes were treated with Ad-GFP or mutant Ad-CRTC2^{K628R}; 48 h later (FIG. 6I), CRTC2 protein expression was evaluated via Western blot, (FIG. 6J) glucose production was measured after treatment with glucagon (100 nM), forskolin (10 μM) or Bt2-cAMP (100 μM) for 4 h, and mRNA expression of gluconeogenic genes was evaluated after treatment with (FIG. 6K) glucagon, (FIG. 6L) forskolin, or (FIG. 6M) Bt2-cAMP for 0-3 h (n=3). Data are expressed as mean±standard error of the mean (s.e.m.). *p<0.05, **p<0.01, ***p<0.001, NS, not significant (FIG. 6D: unpaired t test; FIGS. 6E-6G, FIGS. 6J-6M: two-way ANOVA).

FIGS. 7A-7J show Sam68 binds CRTC2 and inhibits CRTC2 ubiquitination and proteosomal degradation. In FIG. 7A, WT and Sam68^{-/-} mice were treated with glucagon (30 μg/kg) or PBS for 10 min; then, nuclear and cytoplasmic proteins were isolated from the liver tissue, and Sam68 protein expression was evaluated via Western blot. In FIG. 7B, CRTC2 was immunoprecipitated from WT and Sam68^{-/-} primary hepatocytes, and then CRTC2, Sam68, and CREB protein was detected in the precipitates via immunoblot. In FIG. 7C, 293T cells were co-transfected with plasmids coding for HA-tagged Sam68 and Flag-tagged CRTC2; 48 h later, CRTC2 was immunoprecipitated from the cells with anti-Flag, and Sam68 was detected in the precipitates by immunoblotting with anti-HA. In FIG. 7D, 293T cells were co-transfected with plasmids coding for Flag-tagged CRTC2 and HA-tagged WT Sam68 or HA-tagged mutations of Sam68 lacking the N-terminal domain (ΔN), the CK domain (ΔCK), proline motifs 3 and 4 (ΔP3-P4), or the C-terminal domain (ΔC); 48 h later, Sam68 and the Sam68 mutants were immunoprecipitated with anti-HA, and CRTC2 was detected in the precipitates by immunoblotting with anti-Flag. In FIG. 7E, WT and Sam68^{-/-} primary hepatocytes were treated with cycloheximide (100 μM) for 0-8 h to block new protein synthesis; then, CRTC2 protein levels were evaluated by Western blot, quantified via Image J software, normalized to β-actin levels, and reported as the proportion of the amount present at 0 h. WT and Sam68^{-/-} primary hepatocytes were treated with cycloheximide and (FIG. 7E) MG132 (20 μM), (FIG. 7F) BZM (100 nM), or (FIG. 7G) bafilomycin (100 nM) for 0-16 (MG132, BZM) or 0-8 (bafilomycin) and CRTC2 protein levels were evaluated by Western blot, quantified via Image J software, normalized to β-actin levels, and reported as the proportion of the amount present in WT cells at 0 h. P62/SQSTM1 and LC3BI/II protein levels were also evaluated to indicate autophagic activity. In FIG. 7H, WT and Sam68^{-/-} primary hepatocytes were treated with MG132 (20 μM) for 16 h and CRTC2 was immunoprecipitated from the cells, and ubiquitinated proteins were detected in the immunoprecipitates via immunoblot. In FIG. 7I, HepG2 cells were co-transfected with plasmids coding for Flag-tagged CRTC2, Myc-Flag-tagged COP1 and HA-tagged WT Sam68; 48 h later, Sam68, CRTC2, and COP1 protein levels were evaluated via immunoblotting with anti-HA, anti-CRTC2, and anti-Myc antibodies, respectively. In FIG. 7J, HepG2 cells were co-transfected with three plasmids, one coding for Flag-tagged CRTC2, one for Myc-Flag-tagged COP1, and one for HA-tagged WT Sam68 or for each of the HA-tagged Sam68 truncation mutants; 48 h later, protein levels for CRTC2, COP1, and Sam68 or the Sam68 truncation mutants were evaluated via immunoblotting with anti-CRTC2, anti-Myc, and anti-HA antibodies, respectively.

FIGS. 8A-8F show Sam68 interacts directly with CRTC2. In FIG. 8A, WT and Sam68^{-/-} primary hepatocytes were treated with glucagon (100 nM) or PBS for 30 min, then, Sam68 protein levels were evaluated in nuclear and cytoplasmic fractions by immunoblot. In FIG. 8B, WT and Sam68^{-/-} primary hepatocytes were treated with glucagon (100 nM) for 30 min; then, CRTC2 was immunoprecipitated, and Sam68 was detected in the precipitate by immunoblot. In FIG. 8C, 293T cells were co-transfected with plasmids coding for HA-tagged Sam68 and Flag-tagged CRTC2; 48 h later, Sam68 was immunoprecipitated from the cells with anti-HA, and CRTC2 was detected in the precipitates by immunoblotting with anti-Flag. FIGS. 8D-8F show computational results from text pattern search and hydropathy analyses show that P5 domain in the C-terminus of Sam68 has 87.5% hydropathic complementarity/percent match (PM) and 0.461 degree of complementary hydropathy (DCH) with the N-terminus of CRTC2 (amino acids 77-84) in a palindromic manner (FIGS. 8D and 8E), suggesting a model that P5 domain in Sam68 binds N-terminal nuclear localization domain of CRTC2 (amino acids 77-84) (FIG. 8F).

FIGS. 9A-9F show blood glucose levels and gluconeogenic gene expression mice carrying and N-terminal deletion (ΔN) of SAM68 than in WT mice. Blood glucose levels were measured in Sam68^{ΔN-Tg} and their WT littermates (FIG. 9A) under feeding conditions and after fasting for 5 h or 16 h, and in the (FIG. 9B) pyruvate tolerance test (PTT) (WT, n=8; Sam68^{ΔN-Tg}, n=8) (FIG. 9C) glucagon tolerance test (GcTT) (WT, n=8; Sam68^{ΔN-Tg}, n=8), and (FIG. 9D) glucose tolerance test (GTT) (WT, n=9; Sam68^{ΔN-Tg}, n=8). Sam68^{ΔN-Tg} and WT mice were sacrificed under feeding conditions or after fasting for 16 h; then (FIG. 9E) mRNA and (FIG. 9F) protein expression of gluconeogenic genes, and protein levels of CRTC2, were evaluated in liver tissues. Data are expressed as mean+standard error of the mean (s.e.m) *p<0.05, **p<0.01, ***p<0.001 versus WT mice under the same conditions and at the same time point (FIG. 9A: unpaired t test; FIGS. 9B-9E two-way ANOVA).

FIGS. 10A-10D show generation and metabolic characterization of Sam68^{ΔN-Tg} mice. Experiments were conducted in mice carrying an HA-tagged ΔN-truncated Sam68 mutation Sam68^{ΔN-Tg}) and their WT littermates. In FIG. 10A, the genotypes of WT and Sam68^{ΔN-Tg} mice were confirmed via PCR. In FIG. 10B, expression of the Sam68^{ΔN} mutant protein was evaluated in the liver, heart, and skeletal muscle tissues of Sam68^{ΔN-Tg} and WT mice via immunoblot with anti-HA. In FIG. 100, blood glucose levels during the insulin tolerance test (ITT) were measured i Sam68^{ΔN-Tg} (n=9) and WT (n=11) mice. In FIG. 10D, Sam68^{ΔN-Tg} and WT mice were treated with (+) or without (−) insulin (1 U/kg); 20 minutes later, liver tissues were harvested and protein levels of phosphorylated AKT (at amino acids S473 and T308) and total AKT were evaluated by Western blot (n=3). Data are expressed as mean±standard error of the mean (s.e.m.). *p<0.05, **p<0.01 (two-way ANOVA). FIGS. 11A-11M show hepatic Sam68 inactivation mitigates diabetic hyperglycemia. Sam68 and CRTC2 protein expression was evaluated in the nucleus and cytoplasm of liver cells (FIG. 11A) from WT mice fed a normal diet (ND) and diabetic WT mice fed a HFD for 3 months and (FIG. 11B) from db/db and control db/m mice at age 2-3 months. In FIG. 11C, Sam68, CRTC2, PGC-la, PEPCK, and G6Pase protein levels in the livers of patients with or without diabetes were evaluated via Western blot (left), quantified densitometrically via Image J software, normalized to β-actin levels and expressed relative to the levels in non-diabetic patients (right). db/db (5-week-old) mice were administered AAV8 coding for GFP-labeled murine Sam68 shRNA (db/db;sh-Sam68 mice) or for GFP-labeled scrambled Sam68 shRNA (db/db;sh-Scr mice) (1×10¹² genome copies per mouse); 3 weeks later, Sam68 (FIG. 11D) mRNA and (FIG. 11E) protein levels were evaluated via qRT-PCR (liver, brown fat, skeletal muscle, epididymal fat, heart, spleen, lung and kidney) and Western blot (liver), respectively and (FIG. 11M) plasma total cholesterol was also measured. Blood glucose levels were measured in diabetic, HFD-fed Sam68^{f/f} (Sam68^{f/f};HFD) and Sam68^{LKO} (Sam68^{LKO};HFD) mice and in db/db;sh-Scr and db/db;sh-Sam68 mice (FIG. 11F) under feeding conditions and after fasting for 16 h, and in the (FIG. 11G) pyruvate-tolerance test (PTT; Sam68^{f/f};HFD: n=7, Sam68^{LKO};HFD: n=8, db/db;sh-Scr: n=8, db/db;sh-Sam68: n=8), (FIG. 11H) glucagon-tolerance test (GcTT; Sam68^{f/f};HFD: n=7, Sam68^{LKO};HFD: n=9, db/db;sh-Scr: n=8, db/db;sh-Sam68: n=8), and (FIG. 11I) glucose-tolerance test (GTT; Sam68^{f/f};HFD: n=7, Sam68^{LKO};HFD: n=9, db/db;sh-Scr: n=8, db/db;sh-Sam68: n=8). In FIGS. 11J and 11K, mRNA and protein levels, respectively, of gluconeogenic genes were evaluated in the livers of Sam68^{f/f};HFD and Sam68^{LKO};HFD mice and of db/db;sh-Scr and db/db;sh-Sam68 mice. In FIG. 11L, Sam68 and CRTC2 protein levels were evaluated in the livers of Sam68^{f/f};HFD and Sam68^{LKO};HFD mice. Data are expressed as mean±standard error of the mean (s.e.m.). *p<0.05, **p<0.01, ***p<0.001, NS, not significant (FIG. 11D, FIG. 11F, right panel of FIG. 11C: unpaired t test; FIGS. 11G-11J: two-way ANOVA).

FIGS. 12A-12G show hepatic Sam68 inactivation reduces gluconeogenic gene expression and blood-glucose levels in diabetic mice. mRNA expression of Sam68, CRTC2, and SREBP-1c, and gluconeogenic genes was evaluated via qRT-PCR in the livers of (FIG. 12A) WT mice fed a normal diet (ND) and diabetic WT mice fed a HFD for 3 months and (FIG. 12B) db/db and control db/m mice at age 2-3 months. In FIG. 12C, mRNA expression of Sam68, CRTC2, and gluconeogenic genes was measured in the livers of patients with or without diabetes (n=10 per group). In FIG. 12D, Sam68 protein levels were evaluated in the epididymal fat, brown fat, skeletal muscle, heart, and lungs of db/db;sh-Scr and db/db;sh-Sam68 mice. Blood glucose levels were measured in (FIG. 12E) Sam68^{f/f};HFD and Sam68^{LKO};HFD mice after 5 h of fasting and in (FIG. 12F) Sam68^{f/f};HFD (n=7), Sam68^{LKO};HFD (n=8), db/db;sh-Scr (n=8) and db/db;sh-Sam68 (n=8) mice during the insulin tolerance test (ITT). In FIG. 12G, mice were treated with (+) or without (−) insulin (1 U/kg), and protein levels of phosphorylated AKT (at amino acids S473 and T308) and total AKT were evaluated 20 min later. In FIG. 12H, serum insulin levels in db/db;sh-Scr and db/db;sh-Sam68 at feeding condition. Data are expressed as mean±standard error of the mean (s.e.m.) *p<0.05, **p<0.01, ***p<0.001, NS, not significant (FIGS. 12A-C, 12E, 12H: unpaired t test, FIG. 12F: two-way ANOVA).

FIGS. 13A-13B show that ablation of hepatic Sam68 expression in mice downregulates the expression of cholesterol synthesis genes. Twelve-week-old male Sam68^{f/f} and Sam68LKOmice were fasted for 16 h and refed for 6 h; then, liver tissues were harvested, and mRNA sequencing (30-35 million paired-end 75 bp sequencing reads per sample) was performed on an Illumina NextSeq500 (the UAB Genomics Core Facility). Genes whose levels of expression differed by at least two-fold (Padj<0.05; n=3 per group) between the two groups were evaluated via (FIG. 13A) Ingenuity Pathway Analysis and (FIG. 13B) Network Eligible Molecules Analyses. Pathways with the highest enrichment scores and the heat map of hepatic cholesterol biosynthetic genes are displayed in FIG. 13A left and 13A right, respectively, and the downstream targets of SREBP-2 that participate in cholesterol metabolism and were upregulated (red) or downregulated (green) in Sam68LKOmice are displayed in FIG. 13B. Significance was evaluated via the right-tailed Fisher's exact test.

FIGS. 14A-14B show that deletion of Hepatic Sam68 in mice mitigates HFD-induced hypercholesterolemia. Plasma TC (FIG. 14A) and TG (FIG. 14B) levels were measured in HFD-fed Sam68^{uf} mice and Sam68^{LKO} mice after 16 h fasting and 6 h refeeding. Significance was evaluated via the unpaired two-tailed t test. *p<0.05, **p<0.01.

FIGS. 15A-15H show that ablation of hepatic Sam68 expression in Apoe−/−mice protects against hypercholesterolemia and atherosclerosis. Sam68^{f/f};Apoe−/−mice were generated by crossing Sam68f/fmice with Apoe−/− mice (Jax Lab 002052) and then intravenously injected at age 5 weeks with AAV8-TBG-iCre (Sam68^{f/f}-iCre;Apoe−/−mice) to induce the hepatic-specific Sam68 deletion, or with control AAV8-TBG-eGFP (Sam68^{f/f}-iGFP;Apoe−/−mice); 5×10⁹ genome copies were administered to each mouse. After AAV8 injection, mice were fed a normal diet for three weeks, and Sam68 mRNA (FIG. 15A) and protein (FIG. 15B) abundance was measured in livers from a subset of animals. The remaining animals were switched to the Western diet (42% fat and 0.2% cholesterol; Envigo, TD.88137) for 16 weeks; then, the animals were fasted for 16 h, refed for 6 h, and immediately euthanized. (FIG. 15C) mRNA and (FIGS. 15D-15E) protein abundance of the indicated genes were evaluated in mouse livers. (FIGS. 15F-15G) Total cholesterol (TC; Thermo Fisher, TR13421) and triglyceride (TG; Thermo Fisher, TR22421) levels were measured in mouse sera. (FIG. 15H) Aortas were cut into longitudinal sections, (FIG. 15I) aortic roots were cut into cross-sections, and the sections were stained with (FIGS. 15H-15I) Oil Red O or (FIG. 15J) MOMA-2 antibodies and Hoechst-33342; then, atherosclerotic lesion formation was quantified by measuring the areas of the Oil-Red-O-stained regions, and macrophage infiltration was quantified by measuring the areas of the MOMA-2-stained regions. mRNA abundance was measured via quantitative, real-time, polymerase chain reaction (qRT-PCR); protein abundance was evaluated via Western blot; and positively stained areas were measured with NIH image J software. Data are expressed as mean±standard error of the mean (s.e.m.), Significance was evaluated via the unpaired two-tailed t test. *p<0.05, **p<0.01, ***p<0.001.

### DETAILED DESCRIPTION

As demonstrated by the present disclosure, the elevations in blood glucose associated with T2D occur through a combination of declines in insulin sensitivity, which reduces peripheral glucose uptake, and an increase in hepatic glucose production. Many first-line therapies for T2D target the increase in production, particularly via hepatic gluconeogenesis^{2,42,43,} and the results presented here are the first to show that Sam68 has a key regulatory role in gluconeogenesis and blood glucose maintenance. Global and liver-specific deletions of Sam68 in mice reduced blood glucose levels under feeding and fasting conditions, as well as in the PTT, GcTT, GTT, and ITT, and these declines were accompanied by a drop in CRTC2 protein levels and in the expression of key gluconeogenic regulators that are upregulated by CRTC2 in response to glucagon signaling. Furthermore, hepatic Sam68 expression was upregulated both in patients with diabetes and in two diabetic mouse models (HFD-fed and db/db), and the hyperglycemic phenotype observed in diabetic mice significantly improved when hepatic Sam68 expression was reduced via genetic deletion or shRNA-mediated inhibition. Thus, therapies targeting the expression of Sam68 represent treatments for normalizing blood-glucose levels in patients with diabetes, particularly T2D.

The important role of hepatic CRTC2 in gluconeogenesis and insulin sensitivity has been extensively documented. In humans, CRTC2 polymorphisms are associated with an increased risk for T2D^{44,45}, and when CRTC2 levels were manipulated in mice, the corresponding changes in blood-glucose measurements were consistent with the observations in Sam68-deficient mice: modest elevations of CRTC2 increased blood glucose levels and decreased insulin sensitivity^{15,37}, global or liver-specific CRTC2 deletions reduced blood glucose and increased insulin sensitivity^{15,39,46} and both oligo- and siRNA-mediated CRTC2 inactivation mitigated hyperglycemia in diabetic mice^{47,48}. CRTC2 triggers both glucose production and a compensatory mechanism that elevates insulin levels to promote glucose uptake^{16,37,39}, which support the notion that the reduced serum insulin levels in Sam68^{LKO} mice may be secondary to altered blood glucose. Additionally, CRTC2 has been shown to modulate insulin sensitivity by regulating lipid metabolism³⁸, and interestingly both the global Sam68 deletion³¹ and the loss of hepatic CRTC2 expression activate thermogenesis⁴⁶. Thus multiple mechanisms may contribute to the enhanced insulin sensitivity with hepatic Sam68-deficiency.

The data herein demonstrate that Sam68 interacts with CRTC2 and prevents the COP1-mediated polyubiquitination and proteasomal degradation of CRTC2 in hepatocytes, which is consistent with the observation that COP1 ubiquitinates CRTC2, and that this mechanism is closely associated with CRTC2-mediated gluconeogenic activity^{16,36}. The present disclosure also demonstrates that both the C- and N-termini of Sam68 are required for CRTC2 stabilization: the C-terminal truncation disrupted the Sam68-CRTC2 interaction, and the ΔN mutant failed to increase CRTC2 stability. The role of the C-terminus in binding was also supported by hydropathy modeling, which indicated that the P5 domain of Sam68 is well-matched with the N-terminal nuclear localization domain of CRTC2. However, the N-terminus of Sam68 is not predicted with high probability to interact with CRTC2, suggesting a more complex mechanism and potential involvement of other molecular components. Notably, the results described herein indicate that Sam68 translocates from the cytoplasm to the nucleus in response to glucagon signaling, and evidence from other laboratories suggest that insulin promotes the nuclear export of Sam68 in rat adipocytes^{49,50} Therefore, Sam68 mediated CRTC2 stability may be associated with intracellular transport of Sam68.

As an adaptor protein, Sam68 coordinates various cellular responses to environmental stimuli. Notably, the present disclosure and others have demonstrated its potent effects in mediating TNF-α receptor signaling and NF-κB activation in a number of cell types^{23,2451}. Since T2D are complex conditions where sub-acute and chronic inflammation play a crucial role via production of pro-inflammatory cytokines and activation of major inflammatory pathways, particularly TNF-α and NF-κB^{52,53}, the present disclosure shows that Sam68 may provide a mechanistic link between tissue inflammation and the pathophysiology of diabetic hyperglycemia.

In summary, the present disclosure demonstrates that Sam68 promotes hepatic and gluconeogenesis by interacting with and stabilizing CRTC2 protein. The present disclosure also shows that the hyperglycemic phenotype observed in two mouse models of diabetes declined significantly in response to hepatic Sam68 inactivation, which suggests that novel therapies targeting Sam68 and/or the Sam68-CRTC2 interaction are therapies to normalize blood-glucose levels in patients with diabetes, particularly T2D and offer a treatment for patients with T2D and related conditions.

Sam68 inhibition, gene deletion or downregulation can be achieved by the use of various reagents, including small molecule inhibitors, peptide inhibitors, antibodies, vectors, inhibitory RNAs (such as for example, shRNA, siRNA, microRNA, and antisense-RNA). The present disclosure demonstrates AAV-Sam68 shRNA based inhibition of Sam68 can protect diabetic mice from hyperglycemia. AAV-based gene therapy is gaining acceptance and the FDA has approved many gene therapeutic drugs. For example, the first AAV2-based gene therapeutic drug was approved by FDA in 2017 for treatment of blindness by delivering a functional copy of human retinal pigment epithelium-specific protein 65 kDa (RPE65) cDNA into the subretinal space of both eyes of patient where it delivers RPE65 cDNA to retinal pigment epithelial (RPE) cells. Importantly, the inhibitory siRNA can be delivered without viral vectors.

### Definitions

As used herein, a Sam68-mediated condition is a disease or condition that is caused, at least in part, by a decrease in the activity of Sam68. Such a decrease in activity may be caused by or be the result of inhibiting the expression of Sam68 (i.e., a decreased protein level of Sam68 in a subject). Such a decrease in Sam68 activity may be caused by or be the result of inhibiting an activity and/or function of Sam68. The inhibition may be achieved through the use of small molecule inhibitors, peptide inhibitors, antibodies, vectors, or inhibitory RNAs (such as for example, shRNA, siRNA, microRNA, and antisense-RNA). In one aspect, inhibiting an activity and/or function of Sam68 comprises inhibiting the stabilization of CRTC2 or the function of CRTC2. Representative diseases or conditions treatable by inhibiting the activity of Sam68 include, but are not limited to, diabetes, particularly T2D, obesity, cardiovascular disease, kidney disease, and hyperglycemia.

As used herein, the term “about” refers to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the terms “animal,” “subject” and “patient” as used herein include all members of the animal kingdom including, but not limited to, mammals, animals (e.g., cats, dogs, horses, swine, etc.) and humans. In certain embodiments, the subject is a human.

As used herein, the term “antibody” includes whole antibodies and any antigen binding fragment or single chain thereof. Examples of an antibody include, but are not limited to, monoclonal antibodies, polyclonal antibodies, multispecific antibodies (for example, bispecific antibodies) formed from at least two intact antibodies or antigen binding fragments thereof, humanized antibodies, chimeric antibodies, anti-idiotypic (anti-Id) antibodies, intrabodies, and antigen binding fragments of any of the foregoing, Whole antibodies are glycoproteins comprising at least two heavy (H) chains and two light (L) chains inter-connected by disulfide bonds. Each heavy chain is comprised of a heavy chain variable (V_{H}) region and a heavy chain constant (C_{H}) region. The C_{H} region is comprised of three domains, C_{H1}, C_{H2} and C_{H3}. Each light chain is comprised of a light chain variable (V_{L}) region and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} region is composed of three CDRs and four FRs, arranged from amino-terminus to carboxy-terminus in the following order: FR1, CDR1, FR2, CDR2, FR3, CDR3, FR4. The V_{H} and V_{L} regions form a binding domain that interacts with an antigen in an antigen-specific manner. The C_{H} and C_{L} regions mediate binding of the antibody to host tissues, cells or factors, including various cells of the immune system (e.g., effector cells) and the first component (Clq) of the classical complement system. Antibodies may be of any type, including IgG, IgE, IgM, IgD, IgA and IgY and of any class, including, class IgG1, IgG2, IgG3, IgG4, IgA1 and IgA2 or subclass.

As used herein, the terms “antigen-binding fragment” or “antigen-binding portion” refer to one or more fragments derived from an antibody described herein (a parent antibody) that retain the ability to specifically bind to the same antigen as the parent antibody. Examples of binding fragments include, but are not limited to, a Fab fragment (a monovalent fragment consisting of the V_{L}, V_{H}, C_{L} and C_{H} domains, a F(ab)2 fragment (a bivalent fragment comprising two Fab fragments linked by a disulfide bridge at the hinge region), a Fab′ fragment (an Fab fragment comprising a portion of the hinge region), a F(ab′)2 fragment (a bivalent fragment comprising two Fab fragments linked by a disulfide bridge at the hinge region and containing a portion of the hinge region) a Fd fragment (a monovalent fragment consisting of the V_{H} and C_{H} domains), a Fv fragment (a monovalent fragment consisting of the V_{L} and V_{H} domains of a single arm of an antibody), a disulphide-linked Fv (sdFv), a dAb fragment (a monomeric fragment consisting of a V_{H} domain), an isolated CDR, a nanobody or single domain antibody (a monomeric fragment consisting of a single variable antibody domain), a portion of the V_{H} region containing a single variable domain and two constant domains, a diabody (an antibody fragments with two antigen-binding sites; such diabody may be bivalent or bispecific), a triabody (an antibody fragments with three antigen-binding sites; such triabody may be trivalent or trispecific), and a tetrabody (an antibody fragments with four antigen-binding sites; such tetrabody may be tetravalent or tetraspecific). The terms also include single chain Fv (scFv) which are created by recombinantly joining the V_{H} and V_{L} genes by a synthetic linker and expressed as a single polypeptide. Examples of scFv include, but are not limited to, scFv-FC, scFv-CH, scFab, and scFv-zipper. An antigen-binding fragment as described herein may be obtained using conventional methods known in the art and tested for binding as is done with conventional whole antibodies. Suitable antigen-binding fragments are described in Pluckthun (The Pharmacology of Monoclonal Antibodies, vol. 113, Rosenburg and Moore eds., (Springer-Verlag, New York), pp. 269-315 (1994)), Hudson et al., Nat. Med. 9:129-134 (2003), WO 93/16185, U.S. Pat. Nos. 5,571,894 and 5,587,458.

As used herein, the term “antisense oligonucleotide” refers to a sequence of subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:antisense oligomer heteroduplex within the target sequence. The subunits may be based on ribose or another pentose sugar or, in a preferred embodiment, a morpholino group. An antisense oligomer can be designed to block or inhibit translation of mRNA or to inhibit natural pre-mRNA splice processing, and may be said “to target” or to be “targeted against” a target sequence with which it hybridizes.

As used herein, the term an “effective amount” or “therapeutically effective amount” refers to an amount of therapeutic compound administered to a subject, which is effective to produce a desired physiological response and/or therapeutic effect in the subject. The actual dose which comprises the effective amount may depend upon the route of administration, the size and health of the subject, the disorder being treated, and the like. One example of a desired physiological response includes decreased activity of Sam68. Another example of a desired physiological response includes inhibiting the stabilization of CRTC2 or the function of CRTC2. Examples of desired therapeutic effects include, without limitation, improvements in the symptoms or pathology of diabetes, particularly T2D, obesity, cardiovascular disease, kidney disease, and hyperglycemia, reducing the progression of symptoms or pathology of diabetes, particularly T2D, obesity, cardiovascular disease, kidney disease, and hyperglycemia, and slowing the onset of symptoms or pathology of diabetes, particularly T2D, obesity, cardiovascular disease, kidney disease, and hyperglycemia, among others.

The term “excipient” as used herein means a substance formulated alongside the active ingredient of a medication included as a carrier, for the purpose of long-term stabilization, bulking up solid formulations that contain potent active ingredients in small amounts (thus often referred to as bulking agents, fillers, or diluents), or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerns such as by facilitating powder flowability or non-stick properties, in addition to aiding in vitro stability such as prevention of denaturation or aggregation over the expected shelf life. Suitable pharmaceutical excipients are described in “Remington: The Science and Practice of Pharmacy”, Academic Press, Hardcover ISBN: 9780128200070, Editor Hardcover ISBN: 9780128200070, 23^{rd} Edition. Pharmaceutical Sciences” by E. W. Martin.

In some embodiments, the “effective amount” or “therapeutically effective amount” in the context of the present disclosure decreases the activity of Sam68 in a subject by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In some embodiments, the “effective amount” or “therapeutically effective amount” in the context of the present disclosure decreases the stability or protein levels of CRTC2 by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In some embodiments, the “effective amount” or “therapeutically effective amount” in the context of the present disclosure improves the symptoms or pathology of NF-1 or an NF-1 mediated condition, reduces the progression of symptoms or pathology of diabetes, particularly T2D, obesity, cardiovascular disease, kidney disease, and hyperglycemia, and/or slows the onset of symptoms or pathology of diabetes, particularly T2D, obesity, cardiovascular disease, kidney disease, and hyperglycemia, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

In each of the foregoing, when a reduction of increase is specified, such reduction or increase may be determined with respect to a subject that has not been treated with a compound disclosed herein and that has a diagnosis of diabetes, particularly T2D, obesity, cardiovascular disease, kidney disease, and hyperglycemia.

As used herein, the term “pharmaceutically acceptable” refers to a compound that is compatible with the other ingredients of a composition and not deleterious to the subject receiving the compound or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutically acceptable salt” refers to physiologically and pharmaceutically acceptable salts of the compounds of the invention: i.e., salts that retain the desired biological activity of the parent compound and do not impart undesired toxicological effects to the compounds disclosed. For oligonucleotides, preferred examples of pharmaceutically acceptable salts include but are not limited to (a) salts formed with cations such as sodium, potassium, ammonium, magnesium, calcium, polyamines such as spermine and spermidine and the like; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

As used herein, the term “pharmaceutical composition” refers to a mixture of one or more of the compounds of the disclosure, with other components, such as, but not limited to, pharmaceutically acceptable excipients. The purpose of a pharmaceutical composition is to facilitate administration of a compound of disclosure.

As used herein, the term “treatment,” “treating,” or “treat” refers to improving a symptom of a disease or disorder and may comprise curing the disorder, substantially preventing the onset of the disorder, or improving the subject's condition.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

Methods of Treatment and Use

The present disclosure provides for methods of treatment for Sam68 mediated conditions, such as, but not limited to, diabetes, particularly T2D, obesity, cardiovascular disease, kidney disease, and hyperglycemia.

In a first aspect, the present disclosure provides a method of treating a Sam68 mediated condition in a subject, the method comprising administering to a subject a therapeutically effective amount of compound that inhibits an activity of Sam68. In certain embodiments of this aspect, the Sam68 mediated condition is diabetes, particularly T2D, obesity, cardiovascular disease, kidney disease, and hyperglycemia.

In a second aspect, the present disclosure provides a method of treating diabetes, particularly, T2D, in a subject, the method comprising administering to the subject a therapeutically effective amount of compound that inhibits an activity of Sam68.

In a third aspect, the present disclosure provides a method of reducing blood glucose levels in a subject, the method comprising administering to the subject a therapeutically effective amount of compound that inhibits an activity of Sam68.

In a fourth aspect, the present disclosure provides a method of inhibiting gluconeogenesis in a subject, the method comprising administering to the subject a therapeutically effective amount of compound that inhibits an activity of Sam68.

In a fifth aspect, the present disclosure provides a method of activating thermogenesis in a subject, the method comprising administering to the subject a therapeutically effective amount of compound that inhibits an activity of Sam68.

In a sixth aspect, the present disclosure provides a method of decreasing CRTC2 protein levels in a subject, the method comprising administering to the subject a therapeutically effective amount of compound that inhibits an activity of Sam68.

In a seventh aspect, the present disclosure provides a method of increasing the degradation of CRTC2 protein in a subject, the method comprising administering to the subject a therapeutically effective amount of compound that inhibits an activity of Sam68.

In an eighth aspect, the present disclosure provides a method of reducing tissue inflammation in a subject, the method comprising administering to the subject a therapeutically effective amount of compound that inhibits an activity of Sam68.

In a ninth aspect, the present disclosure provides a method of treating obesity in a subject, the method comprising administering to the subject a therapeutically effective amount of compound that inhibits an activity of Sam68.

In a tenth aspect, the present disclosure provides a method of treating a cardiovascular condition in a subject, the method comprising administering to the subject a therapeutically effective amount of compound that inhibits an activity of Sam68.

In an eleventh aspect, the present disclosure provides a method of treating hyperglycemia in a subject, the method comprising administering to the subject a therapeutically effective amount of compound that inhibits an activity of Sam68.

In a twelfth aspect, the present disclosure provides a method of treating kidney disease in a subject, the method comprising administering to the subject a therapeutically effective amount of compound that inhibits an activity of Sam68.

In a thirteenth aspect, the present disclosure provides a method of treating hypercholesterolemia in a subject, the method comprising administering to the subject a therapeutically effective amount of compound that inhibits an activity of Sam68.

In a fourteenth aspect, the present disclosure provides a method of treating atherosclerosis in a subject, the method comprising administering to the subject a therapeutically effective amount of compound that inhibits an activity of Sam68.

In any of the foregoing aspects, compound that inhibits an activity of Sam68 is a small molecule inhibitor, a peptide inhibitor, an antibody, a viral vector expressing an inhibitor RNA, or an inhibitory RNA.

In any of the foregoing aspects, when the compound that inhibits an activity of Sam68 is an inhibitory RNA, the inhibitor RNA may be a shRNA, a siRNA, a microRNA, or an antisense-RNA. In any of the foregoing aspects, when the compound that inhibits an activity of Sam68 is an inhibitory RNA, the inhibitor RNA may be a shRNA.

In any of the foregoing aspects, when the compound that inhibits an activity of Sam68 is an inhibitory RNA, the inhibitor RNA comprises a sequence that is specifically hybridisable to a target sequence in Sam68 RNA or DNA.

In any of the foregoing aspects, when the compound that inhibits an activity of Sam68 is an antibody, the antibody binds the N-terminal domain of Sam68. In any of the foregoing aspects, when the compound that inhibits an activity of Sam68 is an antibody, the antibody binds the C-terminal domain of Sam68. In any of the foregoing aspects, when the compound that inhibits an activity of Sam68 is an antibody, the antibody decreases the association of Sam68 with another protein, such as, but not limited to, CRTC2.

In any of the foregoing aspects, the subject is determined to be in need of treatment. In any of the foregoing aspects, the subject is determined to have diabetes, particularly T2D, obesity, cardiovascular disease, kidney disease, and hyperglycemia (for example, through genetic testing). In any of the foregoing aspects, the subject is suspected to have diabetes, particularly T2D, obesity, cardiovascular disease, kidney disease, and hyperglycemia (for example, through a familial history). In any of the foregoing aspects, the method further comprises administering to the subject a second therapeutic agent useful in the treatment of diabetes, particularly T2D, obesity, cardiovascular disease, kidney disease, and hyperglycemia.

Compositions

Accordingly, the present invention includes a pharmaceutical composition for use in the methods described herein, comprising a compound of the disclosure or pharmaceutically acceptable form thereof, together with a pharmaceutically acceptable excipient suitable for administration to a subject. Such pharmaceutical compositions may further comprise one or more additional active agents. In certain embodiments, the administration route is parenteral, pulmonary, intra-nasal or buccal administration. Such pharmaceutical compositions may be used in the manufacture of a medicament for use in the methods of treatment and prevention described herein. The compounds of the disclosure are useful in both free form and in the form of pharmaceutically acceptable salts.

Pharmaceutically acceptable excipients are also well-known to those who are skilled in the art. The choice of excipient will be determined in part by the particular compound(s), as well as by the particular method used to administer the composition. Accordingly, there is a wide variety of suitable formulations of the pharmaceutical composition of the present invention. The following methods and excipients are merely exemplary and are in no way limiting. Suitable excipients include solvents such as water, alcohol, and propylene glycol, solid absorbants and diluents, surface active agents, suspending agent, tableting binders, lubricants, flavors, and coloring agents. The pharmaceutically acceptable excipients can include polymers and polymer matrices. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. Typically, the pharmaceutically acceptable excipient is chemically inert to the active agents in the composition and has no detrimental side effects or toxicity under the conditions of use. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, for example, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

The compounds of the present disclosure and pharmaceutical compositions containing such compounds as described in the instant disclosure can be administered by any conventional method available for use in conjunction with pharmaceuticals, either as individual therapeutic agents or in combination with additional therapeutic agents.

In one embodiment, the compounds of the present disclosure are administered in therapeutically effective amount, whether alone or as a part of a pharmaceutical composition. The therapeutically effective amount and the dosage administered will, of course, vary depending upon known factors, such as the pharmacodynamic characteristics of the particular agent and its mode and route of administration, the age, health and weight of the recipient; the severity and stage of the disease state or condition; the kind of concurrent treatment; the frequency of treatment; and the effect desired.

The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

In these pharmaceutical compositions, the compound(s) of the present disclosure will ordinarily be present in an amount of about 0.5-95% weight based on the total weight of the composition. Multiple dosage forms may be administered as part of a single treatment.

The active agent can be administered enterally in solid dosage forms, such as capsules, tablets, and powders, or in liquid dosage forms, such as milk, elixirs, syrups and suspensions. It can also be administered parenterally, in sterile liquid dosage forms. The compound(s) of the present disclosure can also be administered intranasally (nose drops) or by inhalation via the pulmonary system, such as by propellant based metered dose inhalers or dry powders inhalation devices. Other dosage forms include topical administration, such as administration transdermally, via patch mechanism or ointment.

Formulations suitable for enteral or oral administration may be liquid solutions, such as a therapeutically effective amount of the compound(s) dissolved in diluents, such as milk, water, saline, buffered solutions, infant formula, other suitable excipients, or combinations thereof. The compound(s) can then be mixed to the diluent just prior to administration. In an alternate embodiment, formulations suitable for enteral or oral administration may be capsules, sachets, tablets, lozenges, and troches. In each embodiment, the formulation may contain a predetermined amount of the compound(s) of the present disclosure, as solids or granules, powders, suspensions and suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients.

Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) can be administered in a physiologically acceptable diluent in a pharmaceutically acceptable excipient, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl .beta.-aminopropionates, and 2-alkylimidazoline quaternary ammonium salts, and (e) mixtures thereof.

The parenteral formulations typically contain from about 0.5% to about 50% by weight of the compound(s) in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

In certain embodiments, the compound of the disclosure is delivered as an aerosols comprising a plurality of solid particles. Such aerosols may be produced by any means known in the art, including but not limited to, an insufflator and a metered dose inhaler. Suitable formulations for administration by insufflation include finely comminuted powders. In one embodiment, the compound of the disclosure is present as a powder and contained in a cartridge which is pierced or otherwise opened to allow the powder to be drawn through the device when a subject inhales or on the activation of a pump. Suitable excipients include, but are not limited to diluents and surfactants as is known in the art. Metered dose inhalers are pressurized aerosol dispensers, typically containing a suspension or solution of the compound of the disclosure in a liquid propellant. Suitable propellants include certain chlorofluorocarbon compounds, for example, dichlorodifluoromethane, trichlorofluoromethane, dichlorotetrafluoroethan-e and mixtures thereof.

The compound(s) of the present disclosure can be formulated into aerosol formulations to be administered via nasal or pulmonary inhalation. These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer.

For nasal or pulmonary delivery, a composition comprising a therapeutically effective amount of a compound of the disclosure is administered to the subject via inhalation into the nose/respiratory system. Such a composition may comprise a particulate form of the compound of the disclosure. The particulate forms of the compound of the disclosure (whether soli or liquid) are produced to be of a size suitable for inhalation therapy such that, for example, the particulate forms of the compound of the disclosure pass through the mouth upon inhalation and into the bronchi and alveoli of the lungs. In one embodiment, a suitable size range for such particulates is in the range of 0.5 to 15 microns. The compound of the disclosure administered by inhalation may be in the form of a dry powder, a mist, or an aerosol.

In certain embodiments, the compound of the disclosure is delivered as an aerosols comprising a plurality of liquid particles. Such aerosols may be produced by any means known in the art, including but not limited to, a nebulizer, a pressure driven nebulizer, and an ultrasonic nebulizer.

The compound(s) of the present disclosure, alone or in combination with other suitable components, may be administered in an aqueous solution as a nasal or pulmonary spray and may be dispensed in spray form by a variety of methods known to those skilled in the art. Systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the active agent dissolved or suspended in a pharmaceutical solvent, e.g., water, ethanol, or a mixture thereof.

Nasal and pulmonary solutions of the present invention typically comprise the drug or drug to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 3.0 and 6.0, preferably 4.5.+−0.0.5. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphatidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, nasal and pulmonary formulations are administered as dry powder formulations comprising the active agent in a dry, usually lyophilized, form of an appropriate particle size, or within an appropriate particle size range, for intranasal delivery. Minimum particle size appropriate for deposition within the nasal or pulmonary passages is often about 0.5 μm. mass median equivalent aerodynamic diameter (MMEAD), commonly about 1 μm MMEAD, and more typically about 2 μm MMEAD. Maximum particle size appropriate for deposition within the nasal passages is often about 10 μm MMEAD, commonly about 8 μm MMEAD, and more typically about 4 μm MMEAD. Intranasally and pulmonaryly respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which relies on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

To formulate compositions for nasal or pulmonary delivery, the active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for nasal or pulmonary delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The compound(s) of the present disclosure may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application. The use of a selected carrier in this context may result in promotion of absorption of the active agent.

The compounds of the present disclosure may alternatively contain as pharmaceutically acceptable excipients substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, magnesium carbonate, and the like.

### EXAMPLES

### Example 1—Sam68 Regulates Blood Glucose Homeostasis by Promoting Hepatic Gluconeogenesis

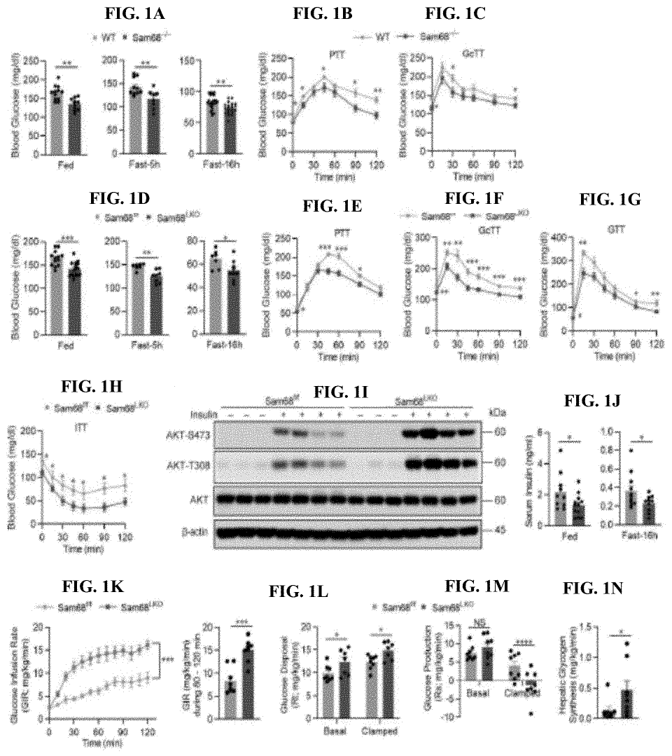

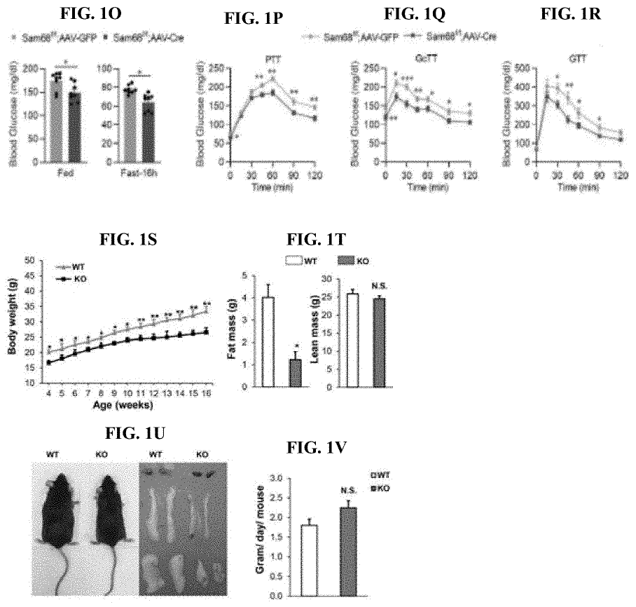

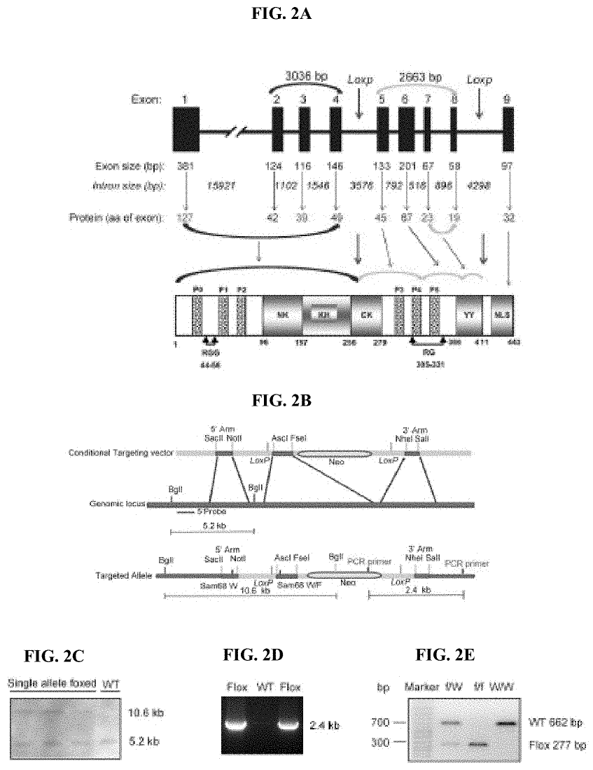

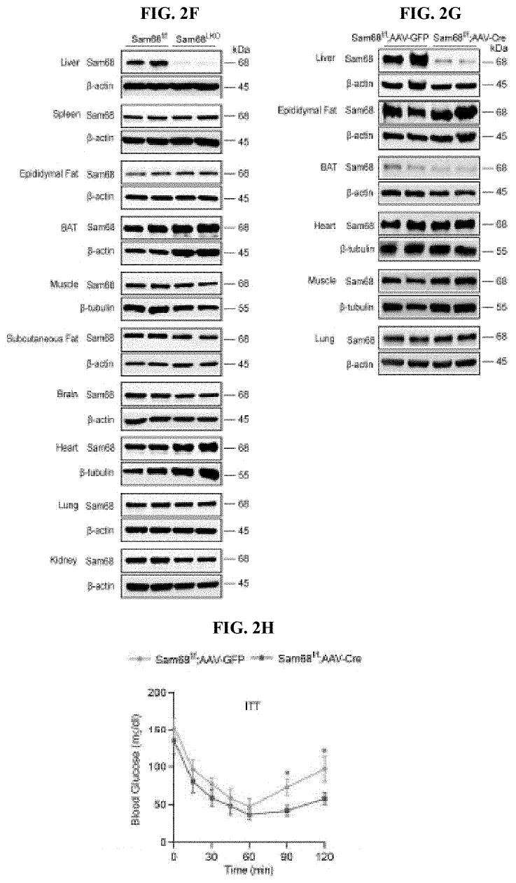

Blood-glucose levels in Sam68^{-/-} mice and their matched WT littermates were compared. Glucose levels were significantly lower in Sam68^{-/-} mice under both feeding and fasting conditions (FIG. 1A), as well as in the pyruvate-tolerance (FIG. 1B) and glucagon-tolerance (FIG. 1C) tests (PTT and GcTT, respectively), both of which measure gluconeogenesis. Since gluconeogenesis occurs primarily in the liver, hepatocyte-specific Sam68 knockout)(Sam68^{LKO}) mice were generated (FIGS. 2A-2E), confirmed that Sam68 protein levels declined in the liver but not in other organs (FIG. 2F), and then repeated Applicants' assessments: blood glucose levels were significantly lower in Sam68^{LKO} mice than in their littermates with normal Sam68 expression (Sam68^{f/f} mice), whether the animals were fed or fasted (FIG. 1D), and when evaluated in the PTT (FIG. 1E) and GcTT (FIG. 1F). Hepatic Sam68 deficiency was also associated with lower blood glucose levels after the injection of glucose (FIG. 1G) or insulin (FIG. 1H) (i.e., in the glucose-tolerance and insulin-tolerance tests, GTT and ITT, respectively), and the amount of phosphorylated AKT (at serine-473 and tyrosine-308) in the liver was greater after insulin treatment in Sam68^{LKO} mice than in Sam68^{f/f} mice (FIG. 1I). Notably, serum insulin concentrations were lower in Sam68^{LKO} mice than in Sam68^{fl/fl} littermates at both feeding and fasting conditions (FIG. 1J). Thus, hepatic Sam68 knockout appears to increase glucose tolerance and insulin sensitivity but reduce insulin levels.

The increase in insulin sensitivity observed in Sam68^{LKO} mice was corroborated via hyperinsulinemic-euglycemic clamp assessments. When mice were systemically infused with insulin, the amount of glucose required to compensate for the increase in insulin levels and prevent hypoglycemia was significantly greater for Sam68^{LKO} mice than for their Sam68^{f/f} littermates (FIG. 1K). Furthermore, since plasma insulin levels in Sam68^{LKO} mice and Sam68^{f/f} mice were similar during clamping, the higher rate of glucose disposal observed in Sam68^{LKO} mice (FIG. 1L) was attributable to differences in insulin sensitivity. Consistent with this whole-body increase in insulin sensitivity, the Sam68^{-/-} mice are leaner than WT mice³¹ and display defects in both adipocyte differentiation³⁰ and male fertility³². To verify that the effect of hepatic Sam68 deficiency on insulin sensitivity and gluconeogenesis was not caused by abnormalities in embryonic or neonatal development that subsequently altered liver function, assessments were conducted after inducing the Sam68 deletion in the hepatocytes of adult Sam68^{f/f} mice via intravenous injection of adeno-associated virus 8 (AAV8) coding for the expression of Cre recombinase (AAV8-TBG-iCre) from the hepatocyte-specific thyroxine-binding globulin (TBG) promoter³³ (Sam68^{f/f};AAV-Cre mice); control assessments were conducted in mice after administration of AAV8 coding for TBG-regulated GFP expression (Sam68^{f/f}; AAV-GFP mice) (FIG. 2G). Three weeks after virus injections, blood glucose levels were significantly lower in Sam68^{f/f};AAV-Cre mice than in Sam68^{f/f};AAV-GFP mice under both feeding and fasting conditions (FIG. 1O), and in the PTT (FIG. 1P), GcTT (FIG. 1Q), GTT (FIG. 1R), and ITT (FIG. 2H). Thus, the results from experiments with Sam68^{f/f};AAV-Cre mice and Sam68^{LKO} mice were consistent and demonstrate the role of hepatic Sam68 expression in systemic glucose homeostasis.

### Example 2—Deletion of Sam68 in Mice Protects Against Diet-Induced Obesity

The body weight and composition in Sam68^{-/-} mice and their WT littermates were measured. Sam68^{-/-} mice display a significantly reduced body weight and fat mass with the difference explained entirely by decreased adiposity, as lean mass is normal (FIGS. 1S-1T). Further analyses show that BAT, inguinal and epididymal depots are smaller in Sam68 mice (FIG. 1U), The effect on adiposity is not mediated by food intake, which did not differ between the two genotypes (FIG. 1V).

### Example 3—Sam68 Promotes Hepatic Gluconeogenesis by Altering Glucagon and Insulin Signaling Balance

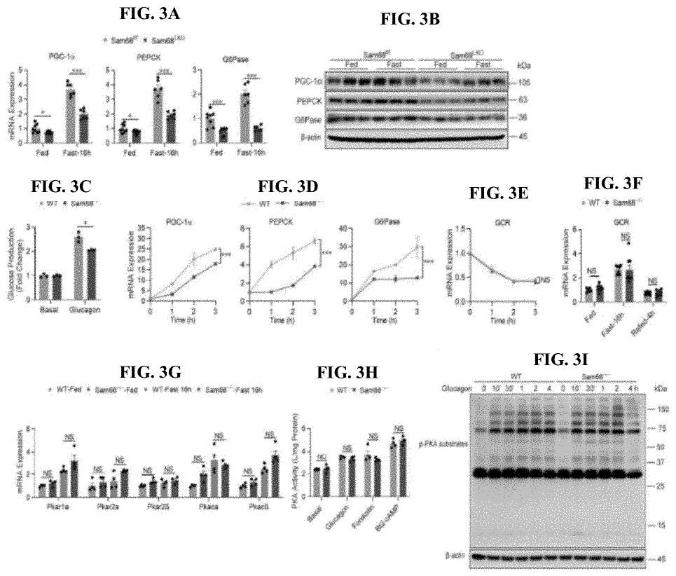

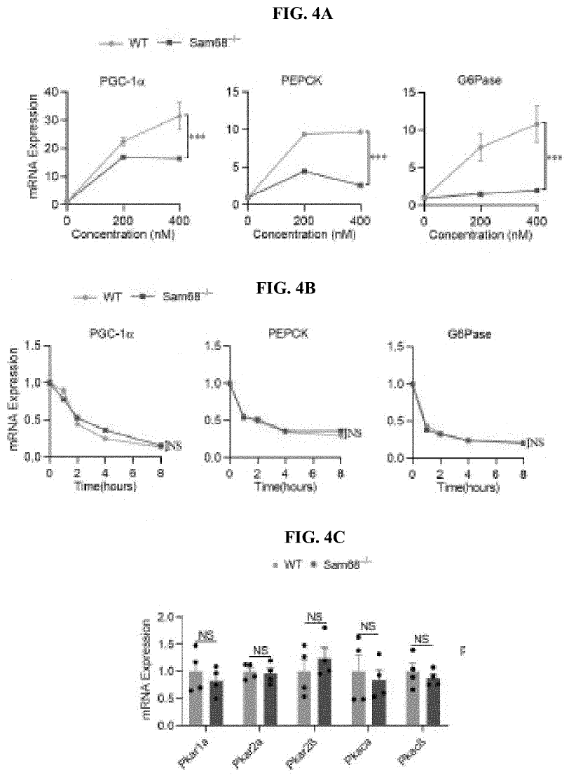

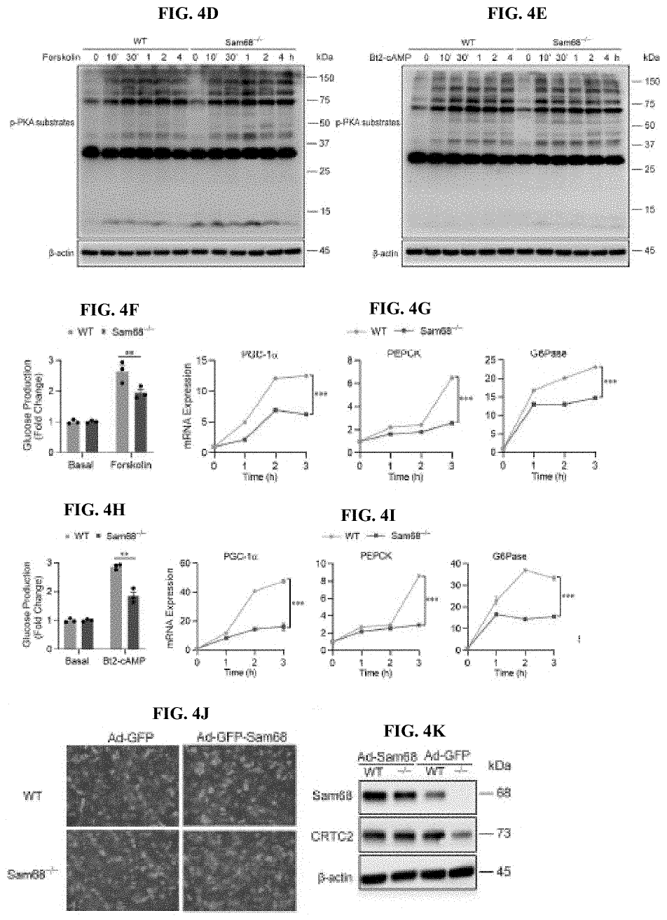

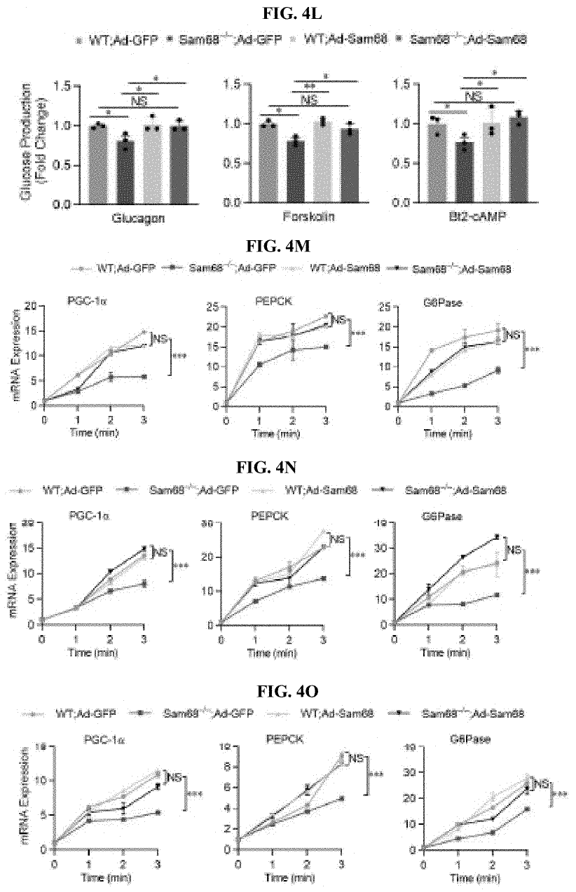

To investigate the molecular mechanism by which Sam68 promotes hepatic gluconeogenesis, mouse liver tissues were examined for the expression of key gluconeogenic genes. Both mRNA (FIG. 3A) and protein (FIG. 3B) levels of PGC-1□, PEPCK, and G6Pase were dramatically lower in Sam68^{LKO} mice than in Sam68^{f/f} mice under feeding and fasting conditions. Furthermore, when primary hepatocytes were isolated from Sam68^{-/-} and WT mice and then treated with glucagon, the Sam68 deletion was associated with significantly lower measures of glucose production (FIG. 3C), and the expression of all three gluconeogenic genes was significantly less upregulated (FIG. 3D, FIG. 4) in Sam68^{-/-} hepatocytes than in WT hepatocytes, with the difference between the two cell populations generally increasing in a time- and glucagon-dose-dependent manner. Notably, Sam68 is an RNA-binding protein, which suggests that it could influence gluconeogenic mRNA and protein levels by modulating mRNA stability. However, when WT and Sam68^{-/-} hepatocytes were sequentially treated with glucagon and Actinomycin D (a specific RNA synthesis inhibitor), the stability of gluconeogenic mRNAs in the two cell populations was similar (FIG. 4B). Thus, the Sam68 deletion appears to reduce gluconeogenic mRNA and protein levels by impeding glucagon signaling, rather than increasing the rate of mRNA degradation.

### Example 4—Sam68 Potentiates Glucagon Signaling by Maintaining CRTC2 Protein Levels

The early steps in glucagon signaling are initiated by the binding of glucagon to the glucagon receptor, which promotes adenylyl cyclase activity. Activated adenylyl cyclase increases cAMP production and cAMP-mediated protein kinase A (PKA) activation³⁴. Sam68 deficiency did not significantly alter glucagon receptor expression (FIGS. 3E-3F) or mRNA levels for any of the subunits³⁵ of the PKA holoenzyme (FIG. 4C, FIG. 4G) in mouse primary hepatocytes or in the livers of mice. Likewise, PKA activity, as well as the phosphorylation of PKA substrates, was similar in WT and Sam68^{-/-} hepatocytes after treatment with glucagon, forskolin (an adenylyl cyclase agonist), or Bt2-cAMP (a cAMP analogue) (FIGS. 3H-3I, FIGS. 4D-4E). Nevertheless, both glucose production and the expression of gluconeogenic genes were significantly less upregulated in Sam68^{-/-} hepatocytes than in WT hepatocytes after treatment with forskolin (FIGS. 3F-3G) or Bt2-cAMP (FIGS. 4H-4I), while transfecting Sam68^{-/-} hepatocytes with an adenoviral vector coding for a Sam68-GFP fusion protein (FIGS. 4J-4K) restored glucagon-, forskolin-, and Bt2-cAMP-induced glucose production and gluconeogenic gene expression to near-WT levels (FIGS. 4L-4O). Thus, the upstream portion of the glucagon signaling pathway (i.e., from receptor binding through PKA activation) appears to be functionally isolated from Sam68.

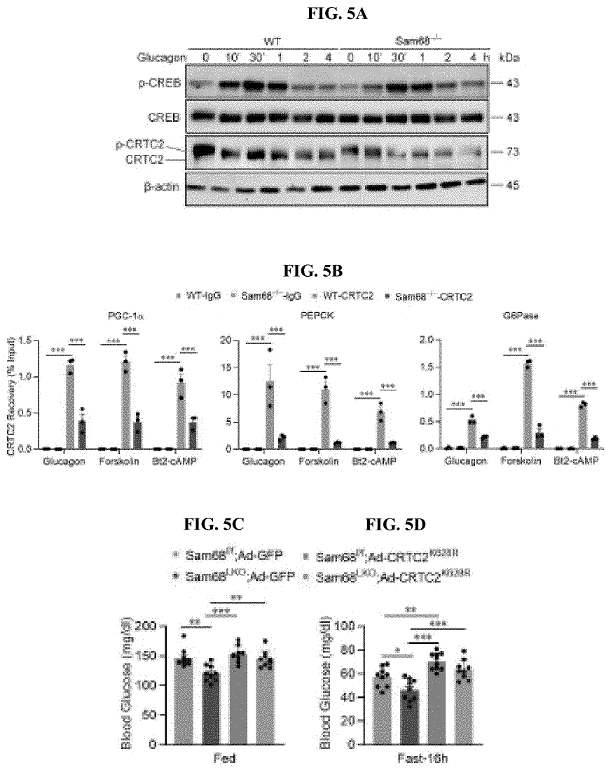

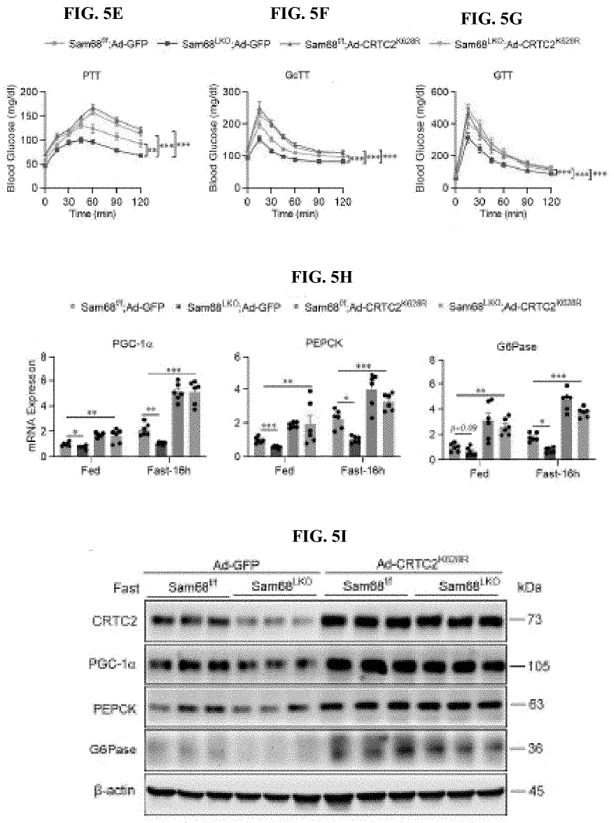

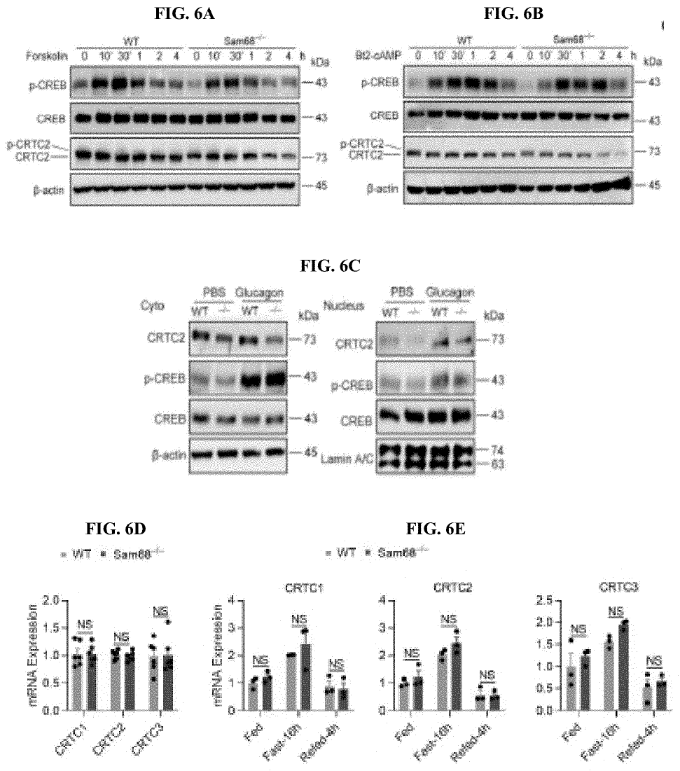

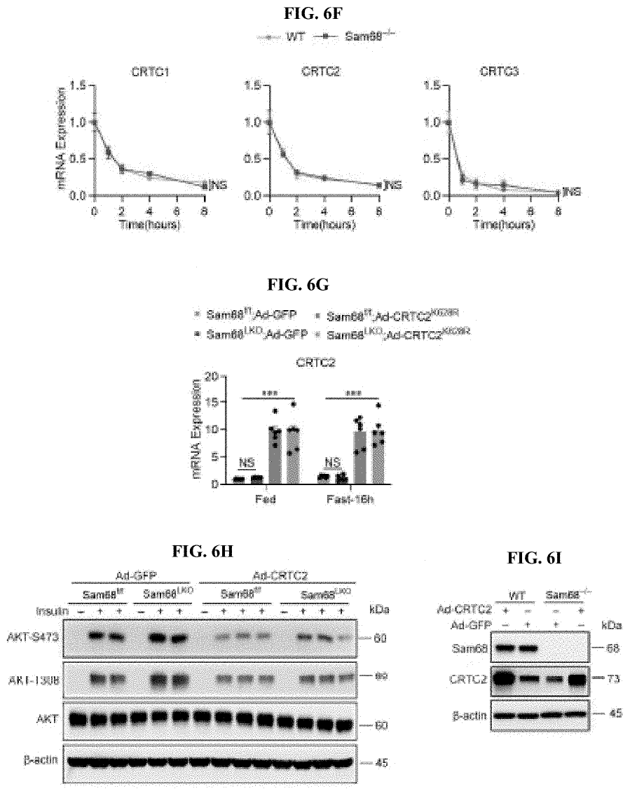

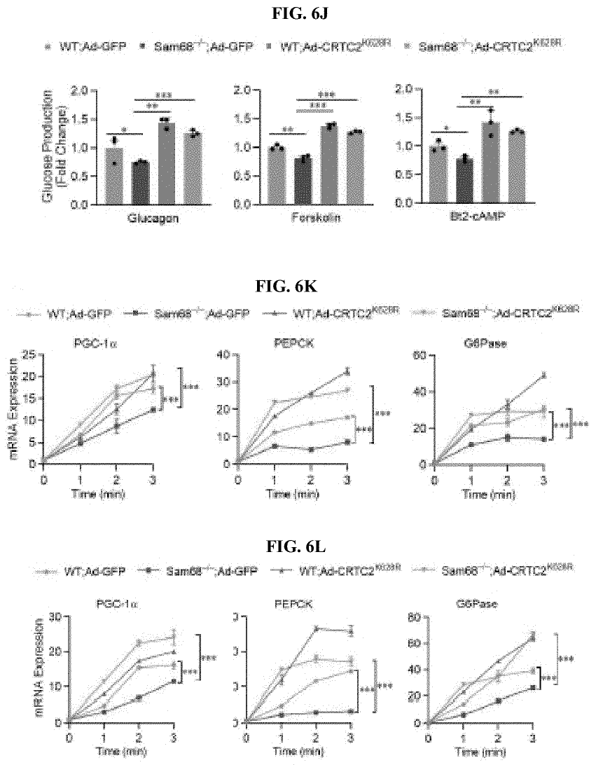

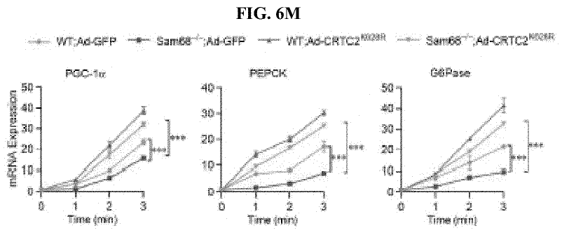

The downstream components of glucagon signaling include CREB and CRTC2, which are phosphorylated and dephosphorylated, respectively, in response to PKA activation. Dephosphorylated CRTC2 translocates into the nucleus, where it enhances the transcriptional activity of phospho-CREB and gluconeogenic gene expression¹⁴. The Sam68 deletion did not substantially change phosphorylated or dephosphorylated CREB protein levels in hepatocytes, but CRTC2 protein levels (for all phosphorylation states) were lower in Sam68^{-/-} than in WT hepatocytes after treatment with glucagon (FIG. 5A), forskolin (FIG. 6A), or Bt2-cAMP (FIG. 6B). The amount of CRTC2 protein was also lower in the cytosolic and nuclear fractions of Sam68^{-/-} hepatocytes than in the corresponding fractions of WT hepatocytes at baseline and after glucagon treatment (FIG. 6C). Chromatin immunoprecipitation (ChIP) assays confirmed that the lower CRTC2 protein levels observed in Sam68^{-/-} hepatocytes were accompanied by dramatic declines in CRTC2 occupancy at the promoters of PGC-1α, G6Pase, and PEPCK after treatment with glucagon, forskolin, or Bt2-cAMP (FIG. 5B). However, the Sam68 deletion did not alter mRNA levels for any of the three CRTC isoforms (CRTC1, CRTC2, and CRTC3) in hepatocytes (FIG. 6D) or in mouse liver tissues under feeding, fasting, or refeeding conditions (FIG. 6E), and measures of CRTC1, CRTC2, and CRTC3 mRNA stability in Sam68^{-/-} and WT hepatocytes were similar (FIG. 6F).

To confirm that the observed declines in CRTC2 protein levels contributed to the downregulation of glucagon signaling and gluconeogenesis in Sam68-deficient mice, experiments were conducted in Sam68^{LKO} mice that had been injected with an adenovirus coding for a degradation-resistant variant of CRTC2 (Ad-CRTC2^{K628R}), which contained a Lys628Arg mutation at its major ubiquitination site³⁶ and has been shown to upregulate glucagon- and cAMP-agonist-induced gluconeogenic gene expression and glucose production in WT hepatocytes^{16,36}. The vector was administered to Sam68^{LKO} mice via tail-vein injection (i.e., in the Sam68^{LKO};Ad-CRTC2^{K628R} group), and comparative assessments were conducted in Sam68^{LKO} mice treated with a GFP-encoding adenovirus (Ad-GFP) and in Sam68^{f/f} mice treated with Ad-CRTC2^{K628R} or Ad-GFP (i.e., the Sam68^{LKO};Ad-GFP, Sam68^{f/f};Ad-CRTC2^{K628R}, and Sam68^{f/f};Ad-GFP groups, respectively) (FIG. 6G). Under feeding (FIG. 5C) and fasting conditions (FIG. 5D), as well as in the PTT, GcTT, and GTT (FIGS. 5E-5G), blood glucose measurements in Sam68^{LKO};Ad-CRTC2^{K628R} mice were significantly greater than those in Sam68^{LKO};Ad-GFP mice and did not differ significantly from measurements in Sam68^{f/f};Ad-CRTC2^{K628R} or Sam68^{f/f};Ad-GFP mice. CRTC2^{K628R} expression also reversed the declines in mRNA (FIG. 5H) and protein (FIG. 5I) levels of gluconeogenic genes, as well as the increase in insulin-induced hepatic AKT phosphorylation (FIG. 6H), observed in Sam68^{LKO} mice, which is consistent with the role of CRTC2 in insulin sensitivity³⁷⁻³⁹, and when CRTC2^{K628R} was expressed in Sam68^{-/-} hepatocytes (FIG. 6I), measures of glucagon-, forskolin-, and Bt2-cAMP-induced glucose production (FIG. 6J) and gluconeogenic gene expression (FIGS. 6K-6M) increased significantly.

Collectively, these observations indicate that the Sam68 deletion impedes glucagon signaling, reduces gluconeogenesis, and improves insulin sensitivity by reducing CRTC2 protein (but not mRNA) levels and the CRTC2-mediated activation of gluconeogenic gene expression.

### Example 5—Sam68 Interacts with CRTC2 and Suppresses CRTC2 Degradation

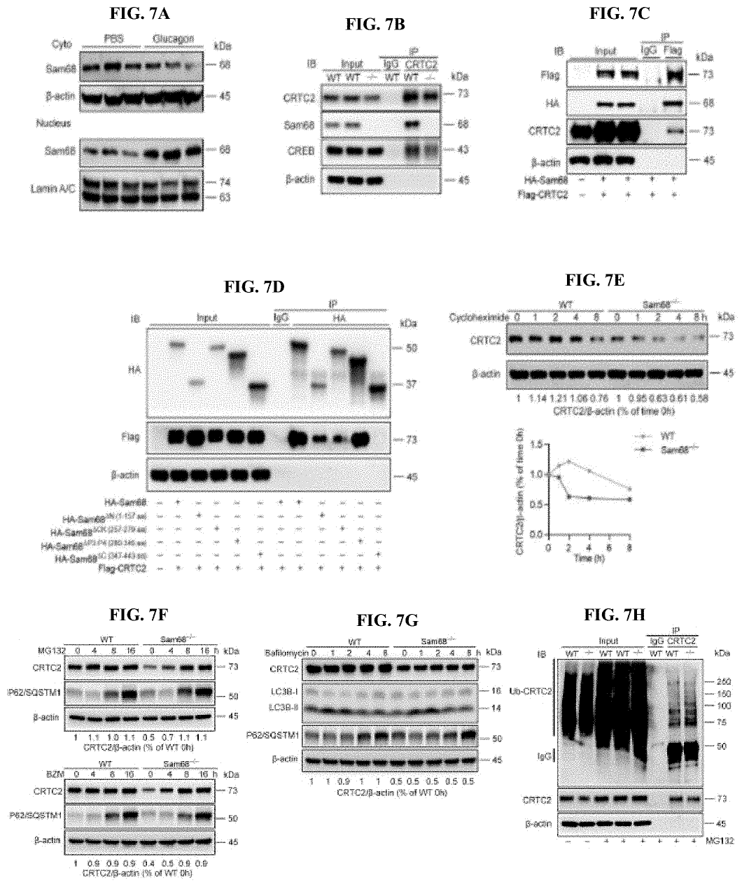

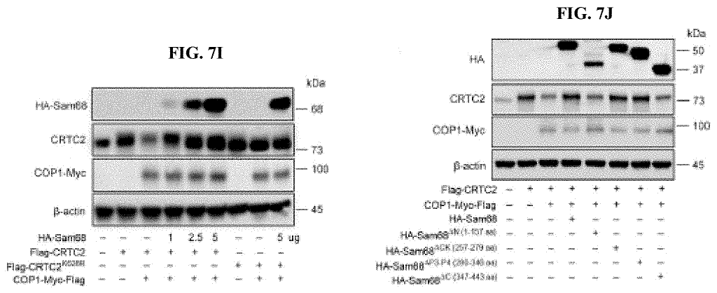

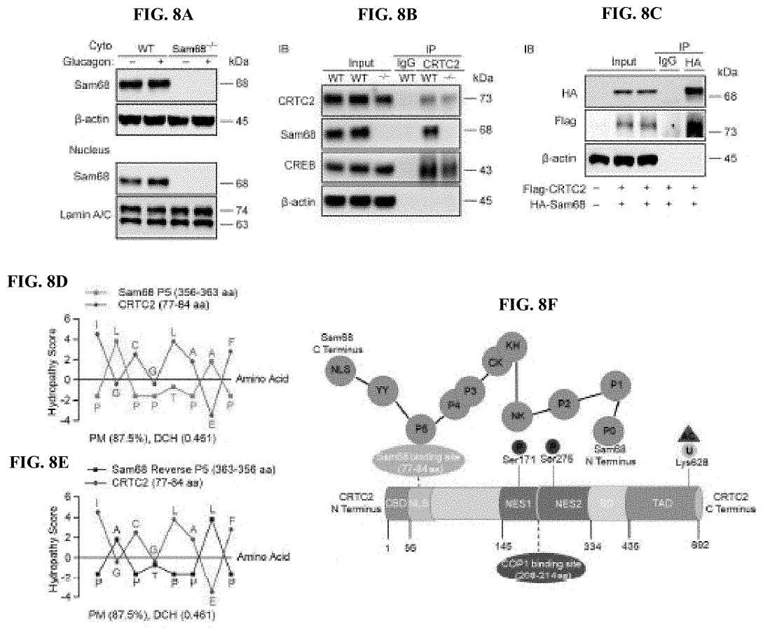

Because glucagon signaling induces gluconeogenesis, in part, by promoting the nuclear translocation of CRTC2¹⁴, it was determined whether glucagon also altered the subcellular distribution of Sam68 in the livers of WT mice (FIG. 7A) and in cultured WT hepatocytes (FIG. 8B). The total amount Sam68 protein was unaltered, but nuclear levels dramatically increased, in response to glucagon treatment. Furthermore, Sam68 mediates a number of biological processes by functioning as an adaptor protein that interacts with other signaling molecues⁴⁰, and co-immunoprecipitation (co-IP) experiments confirmed that endogenous CRTC2 interacted with Sam68 in WT mouse primary hepatocytes at baseline (FIG. 7B) or with glucagon treatment (FIG. 8B), and that exogenous Sam68 and CRTC2 interacted in 293T cells that had been co-transfected with plasmids coding for hemagglutinin (HA)-tagged Sam68 (HA-Sam68) and FLAG-tagged CRTC2 (Flag-CRTC2) (FIG. 7C, FIG. 8C). The specific domains of Sam68 interact with CRTC2 were identified by conducting co-IP experiments in 293T cells that had been co-transfected with Flag-CRTC2 and with plasmids coding for HA-tagged Sam68 truncation mutants lacking the N-terminal domain (amino acids 1-157, Sam68^{ΔN}), the CK domain (amino acids 257-279, Sam68^{ΔCK}), proline motifs 3 and 4 (amino acids 280-346, Sam68^{ΔP3-P4}), or the C-terminal domain (amino acids 347-443, Sam689. Flag-CRTC2 failed to bind HA-Sam68^{ΔC} and interacted much less strongly with HA-Sam68^{ΔN} and HA-Sam68^{ΔCK} than with full-length HA-Sam68 (FIG. 7D).

The observation that the amount of CRTC2 protein, but not mRNA, was significantly downregulated in Sam68^{-/-} hepatocytes suggests that the interaction between Sam68 and CRTC2 reduces the rate of CRTC2 degradation. This hypothesis was tested by measuring CRTC2 protein levels in WT and Sam68^{-/-} hepatocytes after the cells had been treated with cycloheximide for 0-8 hours to inhibit new protein synthesis. CRTC2 protein levels declined more rapidly in Sam68^{-/-} hepatocytes (FIG. 7E), which suggests that Sam68 promotes CRTC2 protein stability. Furthermore, since protein degradation occurs primarily through the ubiquitin-proteasome system or the autophagy-lysosome pathway⁴¹, CRTC2 protein levels were monitored in WT and Sam68^{-/-} hepatocytes after up to 16 hours of treatment with each of two proteasome inhibitors, MG132 or BZM, and up to 8 hours of treatment with the autophagy inhibitor bafilomycin. CRTC2 protein levels approximately doubled in Sam68^{-/-} hepatocytes during the MG132- and BZM-treatment periods (FIG. 7F), but were unchanged by bafilomycin (FIG. 7G), and none of the three treatments altered CRTC2 protein levels in WT hepatocytes by more than ˜10%. These results were corroborated by immunoprecipitating CRTC2 from WT and Sam68^{-/-} hepatocytes after 16 hours of treatment with MG132 (when the total amount of CRTC2 protein in the two cell populations was equivalent), and then measuring the amount of ubiquitinated protein in the precipitate. Polyubiquitinated CRTC2 protein levels were greater in Sam68^{-/-} cells than in WT cells (FIG. 7H). Thus, Sam68 appears to promote CRTC2 stability by impeding CRTC2 ubiquitination and proteosomal degradation.

The ubiquitination of CRTC2 is primarily mediated by the E3 ligase constitutive photomorphogenic protein 1 (COP1)^{15,36,} and when HepG2 cells were co-transfected with three plasmids, one coding for Myc-tagged COP1 (COP1-Myc), one for Flag-CRTC2, and one for HA-Sam68, with the HA-Sam68 plasmid delivered in progressively greater amounts, CRTC protein levels grew as the amount of HA-Sam68 protein increased (FIG. 7I). Notably, CRTC2 protein levels remained largely stable in cells co-transfected with COP1-Myc, with a plasmid coding for a Flag-tagged version of the degradation-resistant CRTC2 variant (Flag-CRTC2^{K628R}), and with or without HA-Sam68. To determine which domain of Sam68 mediates the decline in COP1-induced CRTC2 ubiquitination, CRTC2 protein levels were evaluated in HepG2 cells that had been co-transfected with COP1-Myc and Flag-CRTC2, and with HA-Sam68 or each of the HA-tagged Sam68 truncation mutants. Both Sam68^{ΔN} and Sam68^{ΔC}. mutations were associated with a substantial decline in the amount of intact CRTC2 protein (FIG. 7J). Since the interaction between Sam68 and CRTC2 is primarily mediated by the C-terminus of Sam68 (FIG. 7D), which is preserved in the Sam68^{□N} mutant, the N-terminal truncation (ΔN) appears to be a dominant-negative mutation that competes with the WT Sam68 protein for CRTC2 binding but fails to impede COP1-induced ubiquitination. Furthermore, computational results from combined text pattern search and hydropathy analyses showed that P5 domain located near the C-terminus of Sam68 has 87.5% hydropathic complementarity and 0.461 degree of complementary hydropathy with N-terminal nuclear localization domain of CRTC2 (amino acids 77-84) in a palindromic manner (FIGS. 8D-8E), suggesting that P5 domain in Sam68 likely binds amino acids 77-84 in CRTC2 (FIG. 8F).

### Example 6—Sam68^{ΔN} Transgenic Mice Mimic the Changes in Glucose Metabolism and Insulin Sensitivity Observed in Sam68^{-/-} Mice

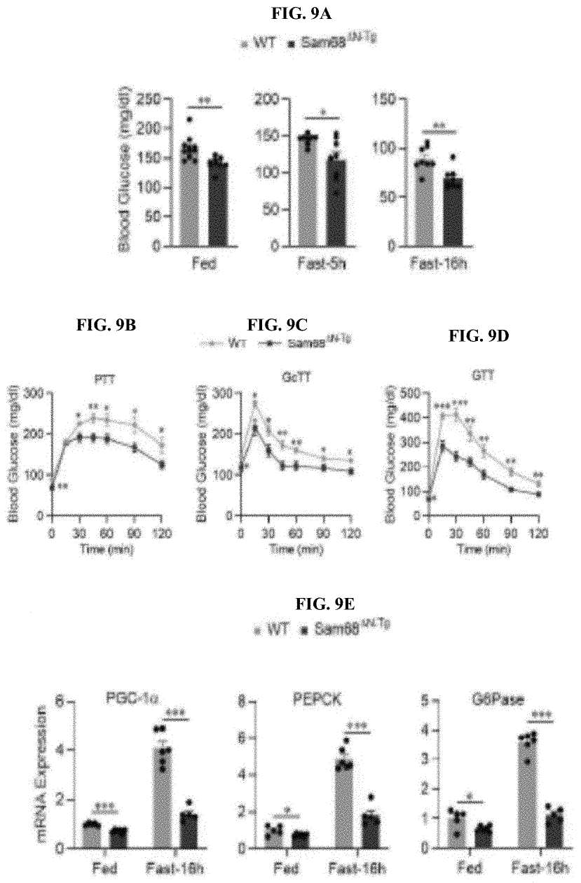

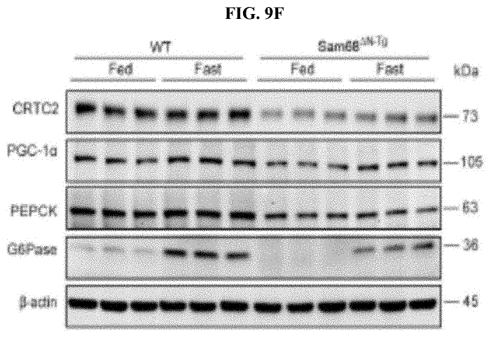

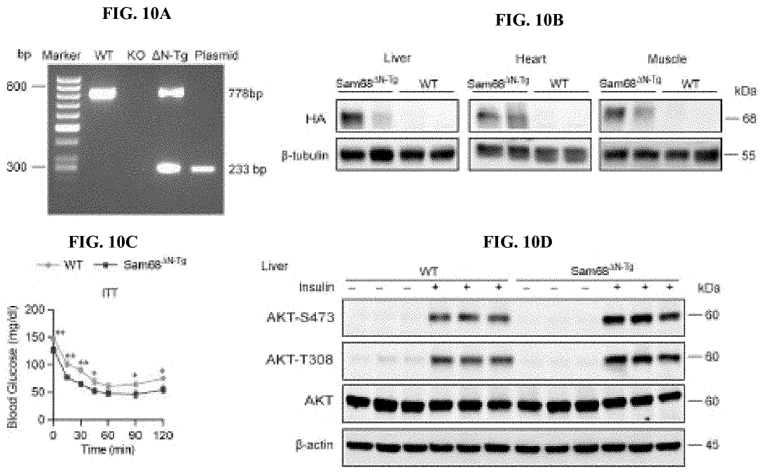

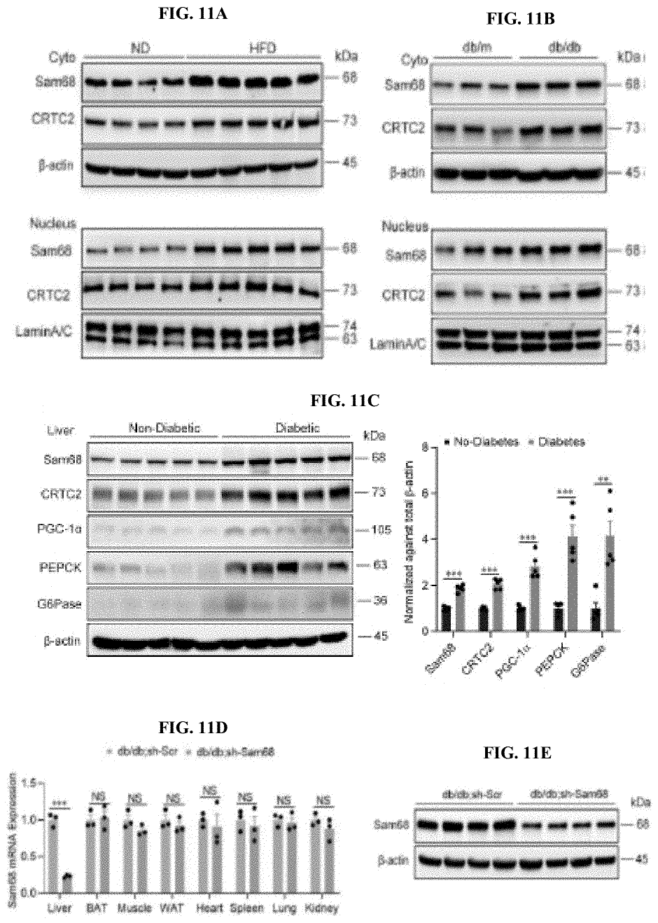

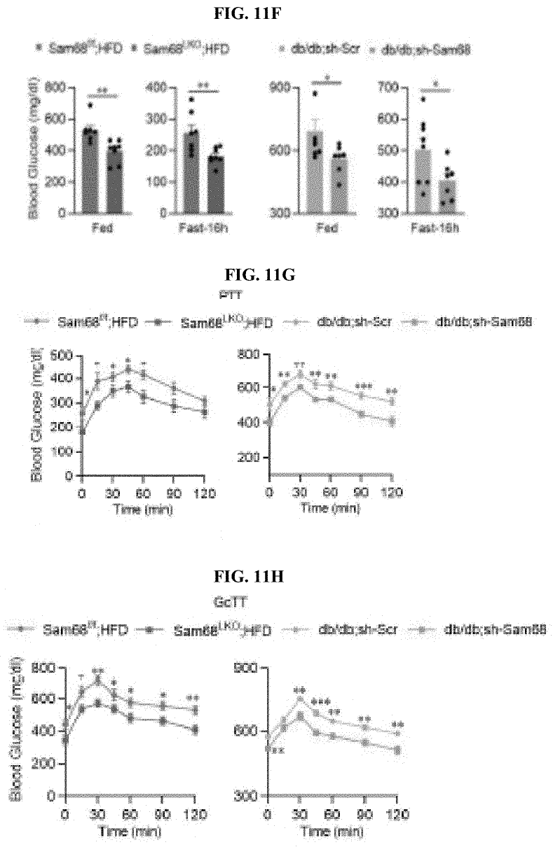

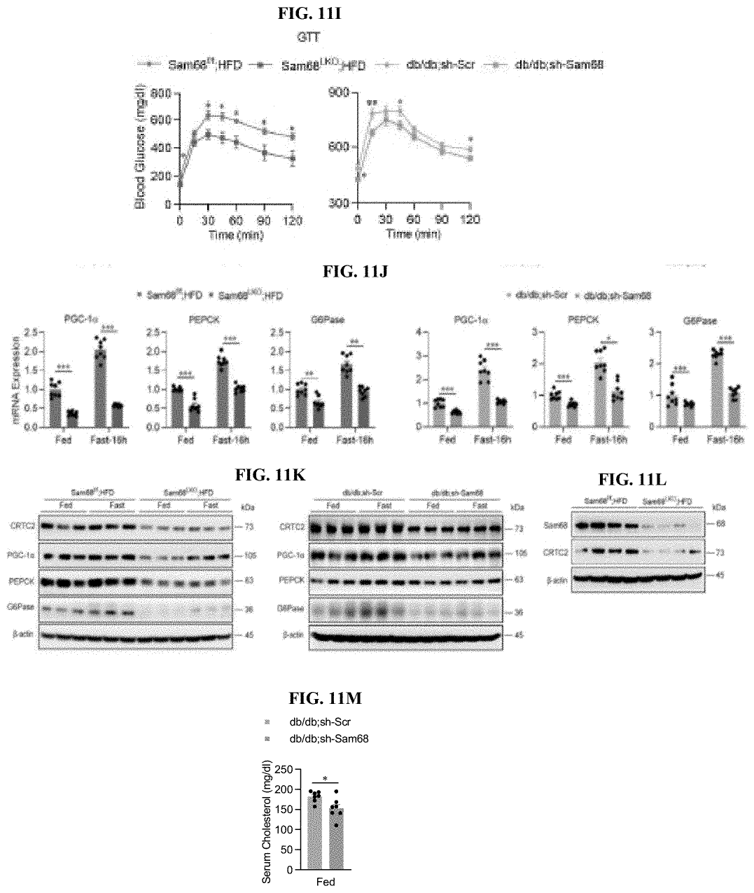

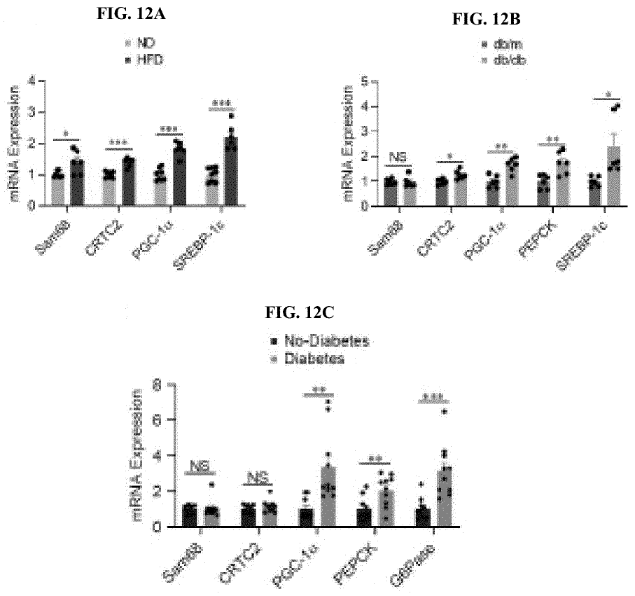

Because the Sam68^{ΔN} truncation disrupted Sam68-CRTC2 binding and reduced CRTC2 stability, it was determined whether the changes in blood-glucose levels, gluconeogenic gene expression, and CRTC2 protein levels observed in Sam68^{-/-} and Sam68^{LKO} mice were reproduced in a line of transgenic, Sam68^{ΔN} mutant (Sam68^{ΔN-Tg}) mice (FIGS. 10A-10B). Compared to their WT littermates, Sam68^{ΔN-Tg} mice displayed lower blood-glucose levels under fed and fasting conditions (FIG. 9A) and in the PTT, GcTT, GTT, and ITT (FIGS. 9B-9D, FIG. 10C). Declines in fed and fasted mRNA (FIG. 9E) and protein (FIG. 9F) measurements of gluconeogenic gene expression, upregulated hepatic insulin signaling (FIG. 10D), and lower amounts of intact CRTC2 protein (FIG. 9F) were also observed. Thus, Sam68^{ΔN-Tg} mice were phenotypically similar to Sam68^{-/-} and Sam68^{LKO} mice, which emphasizes the importance of the N-terminal domain of Sam68 in glucose metabolism, insulin sensitivity, and glucagon signaling.

### Example 7—Downregulating Hepatic Sam68/CRTC2 Signaling Mitigates Hyperglycemia in Diabetic Mice

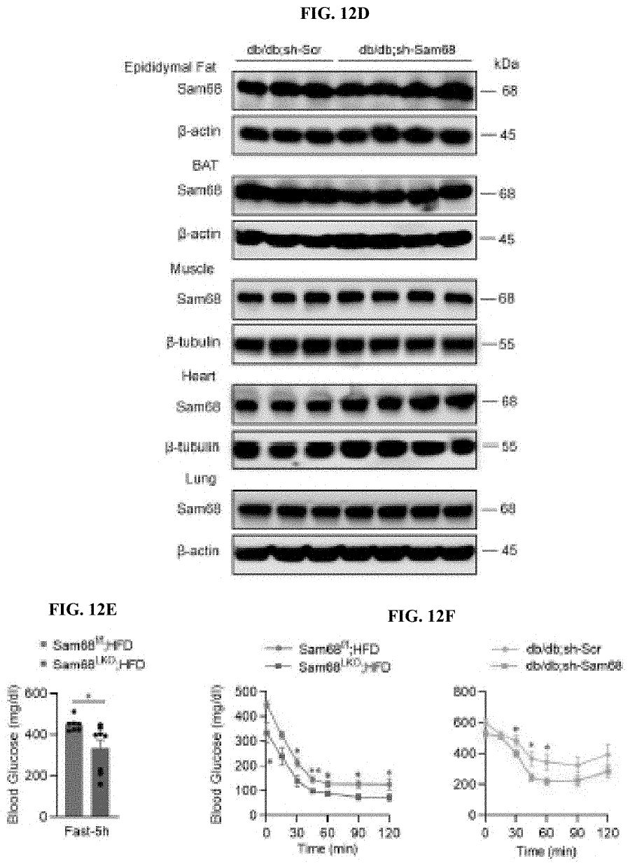

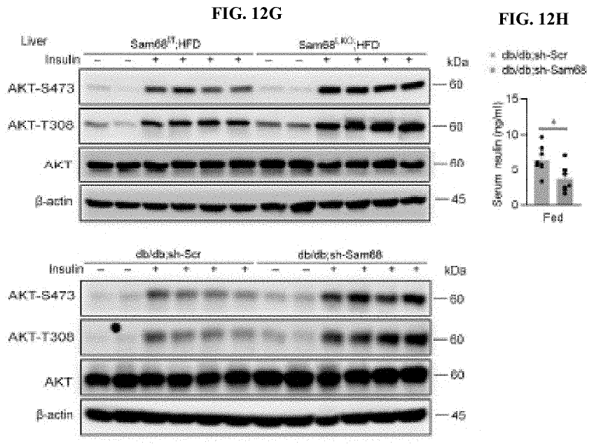

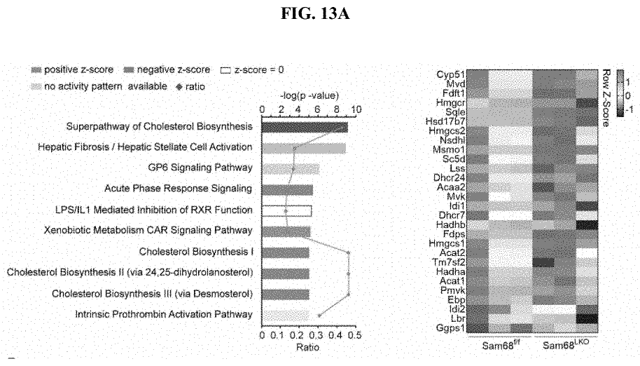

The observation that the Sam68 deletion lowers blood-glucose levels and promotes insulin sensitivity by decreasing the stability of CRTC2 protein suggests that these two molecules may also have a role in the pathogenic mechanisms of diabetes. To test this hypothesis, it was determined whether hepatic Sam68 and/or CRTC2 were upregulated in two commonly used diabetic models, HFD-fed mice and db/db mice, and in human subjects with or without diabetes. In both mouse models, cytosolic and nuclear levels of Sam68 and CRTC2 protein (FIGS. 11A-11B), as well as mRNA measurements of CRTC2 and gluconeogenic gene expression (FIGS. 12A-12B), were significantly greater in liver cells from diabetic than from nondiabetic animals, but only diabetic HFD-fed mouse hepatocytes displayed increases in Sam68 mRNA. Elevated amounts of Sam68 and CRTC2 protein (FIG. 11C), but not mRNA (FIG. 12C), were also observed in liver tissues from patients with diabetes and were accompanied by significant increases in both mRNA and protein levels of gluconeogenic genes.

To confirm that the diabetic phenotypes of HFD-fed and db/db mice could be at least partially attributed to the observed increase in Sam68 expression, experiments were conducted in Sam68^{LKO} and Sam68^{fl/fl} mice that had been fed via the HFD protocol to induce diabetes, and in db/db mice after injection with AAV8 coding for Sam68 shRNA (db/db;sh-Sam68 mice) or a scrambled sequence (db/db;sh-Scr). The Sam68 shRNA reduced Sam68 mRNA and protein levels in the liver by 77% and 70%, respectively, but not in other organs (FIGS. 11D-11E, FIG. 12D). Compared to their corresponding control groups, Sam68^{LKO} and db/db;sh-Sam68 mice had significantly lower blood glucose levels under feeding and fasting conditions (FIG. 11F, FIG. 12E) and in the PTT (FIG. 11G), GcTT (FIG. 11H), GTT (FIG. 11I), and ITT (FIG. 12F), significantly lower mRNA (FIG. 11J) and protein (FIG. 11K) measurements of gluconeogenic gene expression, significantly reduced CRTC2 protein levels (FIGS. 11K-11L), and significant increases in insulin signaling (FIG. 12G) and decreases in serum insulin levels (FIG. 12H). Collectively, these observations demonstrate that the hyperglycemic phenotypes in HFD-fed and db/db mice can be alleviated by downregulating Sam68 expression in the liver and, consequently, that Sam68 represents a novel therapeutic target for the treatment of T2D and related conditions.

Materials and Methods

Animal Studies

All animal experiments in this report were approved by the Institutional Animal Care and Use Committee (IACUC) of the University of Alabama at Birmingham and comply with all relevant ethical regulations, including the National Institutes of Health (NIH) “Guide for the Care and Use of Laboratory Animals”. Experiments were conducted in 2- to 3-month-old, male, C57BL/6 mice unless otherwise specified. Mice were fed ad libitum and maintained under a 12:12-h light:dark cycle.

Genetic Mice

Sam68^{-/-} mice were generated as previously described⁵⁴; db/db and db/+mice were purchased from Jackson Laboratory (No. 000642).

The pGK-Sam68^{floxEx5-8} targeting vector used to generate Sam68^{flox/flox} mice was constructed from the pGKneoF2L2DTA plasmid; the neomycin gene and distal loxP site were inserted into intron 8, and the proximal loxP site was inserted into intron 4 of the Sam68 allele. Briefly, three fragments, a 5′ arm fragment (1736 bp) containing 5′ SacII and 3′ Nott restriction sites, a central fragment (4888 bp) consisting of the floxed allele of exons 5-8 flanked by loxP sites containing 5′ Asc 1 and 3-Fse 1 restriction sites, and a 3′ arm fragment (1771 bp) containing 5′ NheI and 3′ SalI restriction sites, were amplified by PCR from a bacterial artificial chromosome clone (RP24-324H18; BACPAC Resources Center, Oakland Research Institute) that contained the mouse Sam68 gene. The amplified segments were sequentially inserted into the pGKneoF2L2DTA vector via restriction-enzyme digestion and ligation with T4 DNA ligase (NEB, M0202). After verification (by sequencing), the pGK-Sam68^{floxEx5-8} targeting vector was linearized via restriction enzyme (Scat) digestion, purified with a DNA purification kit (Qiagen), and transfected into C57BL/6N embryonic stem (ES) cells. Southern blotting (using a 5′ arm PCR Dig probe that had been synthesized with a commercially available kit [Roche, 11636090910]) and PCR analyses were conducted to confirm that the ES cells had incorporated the floxed Sam68-targeted locus by homologous recombination. The recombinant ES cells were microinjected into blastocysts and implanted into pseudo-pregnant female mice at the Transgenic and Targeted Mutagenesis Laboratory in the Center for Genetic Medicine at Northwestern University. Chimeric off-spring were bred with WT C57Bl/6J mice, and mice that harbored the Sam68^{flox} allele (Sam68^{f/w} mice) in their germline were identified via tail-snip PCR genotyping. The Sam68^{f/w} C57Bl/6N mice were back-crossed to C57Bl/6J mice for six generations to produce the mice used in this report. Sam68^{f/f} mice were generated by interbreeding Sam68^{f/w} heterozygote mice, and mice carrying the hepatocyte-specific knockout of Sam68 were produced by crossing Sam68^{f/f} mice with AIb-cre mice (The Jackson Laboratory, No. 003574) to generate Alb-Cre⁺;Sam68^{f/f}) (Sam68^{LKO} mice and their Sam68^{f/f} littermate controls.

To generate the Sam68^{ΔN} transgenic mice, pcDNA3-HA-Sam68^{ΔN} plasmids were linearized by digestion with restriction enzymes (NruI and StuI) to remove the vector sequence; then, the construct was purified and microinjected into the male pronuclei of C57BL6J oocytes at the Transgenic & Genetically Engineered Model Systems Core Facility of the University of Alabama at Birmingham (UAB). Three of the 4 founders were positive for the transgene (as determined via tail-snip PCR genotyping) and crossed with C57BL/6J mice to verify germline transmission; pups were evaluated via PCR genotyping and Western blotting of liver, heart, and skeletal-muscle tissues with anti-HA.

The sequences of all primers and probes used for vector construction, PCR genotyping, and Southern blotting are reported in Table 1, and the antibodies used for Western blotting are listed in Table 2.

TABLE 1

| | |
|---|---|
| Forward sequence | Reverse sequence |

For assessment of gene expression with qRT-PCR

| | | |
|---|---|---|
| mSREBP-1c | GGAGCCATGGATTGCACATT | GGCCCGGGAAGTCACTGT |
| | SEQ ID NO: 1 | SEQ ID NO: 2 |
| mPGC-1α | AGCCGTGACCACTGACAACGAG | GCTGCATGGTTCTGAGTGCTAAG |
| | SEQ ID NO: 3 | SEQ ID NO: 4 |
| mPEPCK | CCACAGCTGCTGCAGAACA | GAAGGGTCGCATGGCAAA |
| | SEQ ID NO: 5 | SEQ ID NO: 6 |
| mG6pase | TGGGCAAAATGGCAAGGA | TCTGCCCCAGGAATCAAAAAT |
| | SEQ ID NO: 7 | SEQ ID NO: 8 |
| mCRTC1 | CATGATGGAGAACGCCATCAG | CACCGTGAGGATGATGTTGGG |
| | SEQ ID NO: 9 | SEQ ID NO: 10 |
| mCRTC2 | TTTGGGCATCAGTGGAGGTC | CCTGGAGGTTGGGATTGCTT |
| | SEQ ID NO: 11 | SEQ ID NO: 12 |
| mCRTC3 | CACAGTCAGACTTCCAGCTC | ATGGGTTTGGTCTCAAGTGG |
| | SEQ ID NO: 13 | SEQ ID NO: 14 |
| mPkar1α | CCCTCGAGTCAGTACGGATG | GCATTCCTTCGGGAATACTTT |
| | SEQ ID NO: 15 | SEQ ID NO: 16 |
| mPkar2α | CCTCCTCTTCTTCATCAGGG | GAGTGACTCGGACTCGGAAG |
| | SEQ ID NO: 17 | SEQ ID NO: 18 |
| mPkar2β | GATCATCGGTTTTGGGATGT | ATAAACCGGTTCACAAGGCG |
| | SEQ ID NO: 19 | SEQ ID NO: 20 |
| mPkacα | ATTCTGAGAAGGGGTCTCCC | AAGAAGGGCAGCGAGCAG |
| | SEQ ID NO: 21 | SEQ ID NO: 22 |
| mPkacβ | TCCTCAAGCCCAGCATTACT | CAAGAAAGGCAGCGAAGTG |
| | SEQ ID NO: 23 | SEQ ID NO: 24 |
| mGcR | ATTGGCGATGACCTCAGTGTGA | GCAATAGTTGGCTATGATGCCG |
| | SEQ ID NO: 25 | SEQ ID NO: 26 |
| mβ-actin | GTATGGAATCCTGTGGCATC | AAGCACTTGCGGTGCACGAT |
| | SEQ ID NO: 27 | SEQ ID NO: 28 |
| m-Sam68 | GATATCTGTCAGGAGCAGTTTCT | CTCCTCGTCCTCTCACAGATA |
| | SEQ ID NO: 29 | SEQ ID NO: 30 |
| hPGC-1α | AACAGCAGCAGAGACAAATGCACC | TGCAGTTCCAGAGAGTTCCACACT |
| | SEQ ID NO: 31 | SEQ ID NO: 32 |
| hPEPCK | AAGGAGGATGCCCTGAACCTGAAA | TGCACCTTATGGATGGGAAAGGGA |
| | SEQ ID NO: 33 | SEQ ID NO: 34 |
| hG6Pase | TGAATGGCTGCAGTGACCCAGATA | TGGATGTGGAGCCAGTGGAAGAAT |
| | SEQ ID NO: 35 | SEQ ID NO: 36 |
| hCRTC2 | CTCTGCCCAATGTTAACCAGAT | GAGTGCTCCGAGATGAATCC |
| | SEQ ID NO: 37 | SEQ ID NO: 38 |
| hβ-actin | AGGATGCAGAAGGAGATCACTG | GGGTGTAACGCAACTAAGTCATAG |
| | SEQ ID NO: 39 | SEQ ID NO: 40 |
| h-Sam68 | GCGAGTGCTGATACCTGTCAAG | TCATTGAGCCCTTTCCCAAT |
| | SEQ ID NO: 41 | SEQ ID NO: 42 |
| SREBP-1a | GGCCGAGATGTGCGAACT | TTGTTGATGAGCTGGAGCATGT |
| | SEQ ID NO: 43 | SEQ ID NO: 44 |
| SREBP-2 | GCGTTCTGGAGACCATGGA | ACAAAGTTGCTCTGAAAACAAATCA |
| | SEQ ID NO: 45 | SEQ ID NO: 46 |
| SCAP | ATTTGCTCACCGTGGAGATGTT | GAAGTCATCCAGGCCACTACTAATG |
| | SEQ ID NO: 47 | SEQ ID NO: 48 |
| INSIG-1 | TCACAGTGACTGAGCTTCAGCA | TCATCTTCATCACACCCAGGAC |
| | SEQ ID NO: 49 | SEQ ID NO: 50 |
| INSIG-2a | CCCTCAATGAATGTACTGAAGGATT | TGTGAAGTGAAGCAGACCAATGT |
| | SEQ ID NO: 51 | SEQ ID NO: 52 |
| INSIG-2b | CCGGGCAGAGCTCAGGAT | GAAGCAGACCAATGTTTCAATGG |
| | SEQ ID NO: 53 | SEQ ID NO: 54 |
| LXRα | GGATAGGGTTGGAGTCAGCA | CTTGCCGCTTCAGTTTCTTC |
| | SEQ ID NO: 55 | SEQ ID NO: 56 |
| RXRα | ATGGACACCAAACATTTCCTGC | CCAGTGGAGAGCCGATTCC |
| | SEQ ID NO: 57 | SEQ ID NO: 58 |
| HMGCR | AGCTTGCCCGAATTGTATGTG | TCTGTTGTGAACCATGTGACTTC |
| | SEQ ID NO: 59 | SEQ ID NO: 60 |
| HMGCS1 | GCCGTGAACTGGGTCGAA | GCATATATAGCAATGTCTCCTGCAA |
| | SEQ ID NO: 61 | SEQ ID NO: 62 |
| ACAT1 | CAGGAAGTAAGATGCCTGGAAC | TTCACCCCCTTGGATGACATT |
| | SEQ ID NO: 63 | SEQ ID NO: 64 |
| ACAT2 | GACTTGGTGCAATGGACTCG | GGTCTTGCTTGTAGAATCTGG |
| | SEQ ID NO: 65 | SEQ ID NO: 66 |
| DHCR7 | AGGCTGGATCTCAAGGACAAT | GCCAGACTAGCATGGCCTG |
| | SEQ ID NO: 67 | SEQ ID NO: 68 |
| DHCR24 | TTCCCGGACCTGTTTCTGGAT | CTCTGGGTGCGAGTGAAGG |
| | SEQ ID NO: 69 | SEQ ID NO: 70 |
| MVK | CATCGGTATTAAGCAGGTGTGG | GTACCGAGACATCACCTTGCT |
| | SEQ ID NO: 71 | SEQ ID NO: 72 |
| PMVK | GTAGTGGCCTCGGAGCAGA | GTGGTTCTCAATGACCCAGTCA |
| | SEQ ID NO: 73 | SEQ ID NO: 74 |
| MVD | GTCAGTGAACAACTTCCCCACT | ACTTCGGAGAGGTCTCCCTCA |
| | SEQ ID NO: 75 | SEQ ID NO: 76 |
| SQLE | CACAGTTACCTGAGCACCTGA | ACCAGTAAGAGGGTGCCTCA |
| | SEQ ID NO: 77 | SEQ ID NO: 78 |
| CYP51 | CCTTGGCCATGCAATAGCATT | GAAGGTAAGTGAAGGTCTTGCC |
| | SEQ ID NO: 79 | SEQ ID NO: 80 |
| FDFT1 | TCCCTGACGTCCTCACCTAC | CCCCTTCCGAATCTTCACTA |
| | SEQ ID NO: 81 | SEQ ID NO: 82 |
| IDI1 | ACCAGCCATCTTGATGAAAAACA | CAGCAACTATTGGTGAAACAACC |
| | SEQ ID NO: 83 | SEQ ID NO: 84 |
| LSS | TCGTGGGGGACCCTATAAAAC | CGTCCTCCGCTTGATAATAAGTC |
| | SEQ ID NO: 85 | SEQ ID NO: 86 |
| FDPS | GCACTGACATCCAGGACAAC | AGCCACTTTTTCTGGGTCCT |
| | SEQ ID NO: 87 | SEQ ID NO: 88 |
| MSMO1 | ACCATACGTTTGCTGGAAACCATC | AGCGCCCGTATAAAAAGGAACCAA |
| | SEQ ID NO: 89 | SEQ ID NO: 90 |
| HSD17B7 | TCTGTATTCCAGTGTGATGTGC | CTTTTGGCCCGTGACGTAAT |
| | SEQ ID NO: 91 | SEQ ID NO: 92 |
| LDLR | CGCGGATCTGATGCGTCGCT | CGGCCCTGGCAGTTCTGTGG |
| | SEQ ID NO: 93 | SEQ ID NO: 94 |
| APOE | GCTGGGTGCAGACGCTTT | TGCCGTCAGTTCTTGTGTGACT |
| | SEQ ID NO: 95 | SEQ ID NO: 96 |
| PCSK9 | GAGACCCAGAGGCTACAGATT | AATGTACTCCACATGGGGCAA |
| | SEQ ID NO: 97 | SEQ ID NO: 98 |
| SR-BI | AAACAGGGAAGATCGAGCCAG | GGTCTGACCAAGCTATCAGGTT |
| | SEQ ID NO: 99 | SEQ ID NO: 100 |
| APOB | CGTGGGCTCCAGCATTCTA | TCACCAGTCATTTCTGCCTTTG |
| | SEQ ID NO: 101 | SEQ ID NO: 102 |
| APOM | CAGTGCCCTGAGCACAGTCAA | GCTGCTCCCGCAATAAAGTACC |
| | SEQ ID NO: 103 | SEQ ID NO: 104 |
| ABCA1 | GCTTGTTGGCCTCAGTTAAGG | GTAGCTCAGGCGTACAGAGAT |
| | SEQ ID NO: 105 | SEQ ID NO: 106 |
| ABCG1 | CTTTCCTACTCTGTACCCGAGG | CGGGGCATTCCATTGATAAGG |
| | SEQ ID NO: 107 | SEQ ID NO: 108 |
| APOA1 | TATGTGGATGCGGTCAAAGA | ACGGTTGAACCCAGAGTGTC |
| | SEQ ID NO: 109 | SEQ ID NO: 110 |
| APOA2 | ACGGGAAGGACTGCAGCA | GCAGCTTCATGATGGCAGACT |
| | SEQ ID NO: 111 | SEQ ID NO: 112 |
| LCAT | GCCCAAGGCTGAACTCAGTA | AGCTTGGCTTCTAGCCGATT |
| | SEQ ID NO: 113 | SEQ ID NO: 114 |

For ChIP-qPCR

| | | |
|---|---|---|
| mPGC-1α- | GGGCTGCCTTGGAGTGACGTC | AGTCCCCAGTCACATGACAAAG |
| promoter | SEQ ID NO: 115 | SEQ ID NO: 116 |
| mG6Pase- | GGAGGGCAGCCTCTAGCACTGTCAA | TCAGTCTGTAGGTCAATCCAGCCCT |
| promoter | SEQ ID NO: 117 | SEQ ID NO: 118 |
| mPEPCK- | GGCCTCCCAACATTCATTAAC | GTAGCCCGCCCTCCTTGCTTTA |
| promoter | SEQ ID NO: 119 | SEQ ID NO: 120 |

For generation of Sam68f/f mice

| | | |
|---|---|---|
| 5′ arm PCR | CCAAGGCCTCCTCATCTGATG | GTCTACACACAAAGCCCCGAG |
| | SEQ ID NO: 121 | SEQ ID NO: 122 |
| Central | CTGCCCGGCTTCTTGAGTAAG | TCCCCTACTTGTCGGCTCTAC |
| fragment | SEQ ID NO: 123 | SEQ ID NO: 124 |
| 3′ arm PCR | TAGCACCAAGCTCCCTCCAAG | TCCTGTTCCCAACGTCACCAG |
| | SEQ ID NO: 125 | SEQ ID NO: 126 |
| ES screening | GATTCGCAGCGCATCGCCTTCT | TACCGGTGGATGTGGAATGTG |
| PCR | SEQ ID NO: 127 | SEQ ID NO: 128 |
| 5′ arm PCR Dig | CAGGGTTTCTCTGTGTAGCCC | AGTGGCGCACGCCTTTAATCC |
| | SEQ ID NO: 129 | SEQ ID NO: 130 |
| F or W | TTGGGAAAGAGGTATGGCTTGGCA | AAGAAGTTCCTGCCTAACTCTCCC |
| genotyping | SEQ ID NO: 131 | SEQ ID NO: 132 |

For genotyping of Sam68^{LKO} mice

| | | |
|---|---|---|
| Alb-Cre | TGCCTGCATTACCGGTCGATGC | CCATGAGTGAACGAACCTGGTCG |
| | SEQ ID NO: 133 | SEQ ID NO: 134 |

For genotyping of Sam68^{ΔN-Tg} mice

| | | |
|---|---|---|
| Exon6F- | TAGAGGAGCTTTGGTTCGTG | AATAGCCTTCATAGCCTTCG |
| Exon7R | SEQ ID NO: 135 | SEQ ID NO: 136 |

TABLE 2

| Name | Catalogue Number | Company Name |
|---|---|---|
| PGC-lα | ab54481 | Abcam |
| G6Pase | ab83690 | Abcam |
| Sam68 for WB | ab76472 | Abcam |
| PEPCK | 12940 | Cell Signaling |
| β-actin | 8457 | Cell Signaling |
| Lamin A/C | 4777 | Cell Signaling |
| β-tubulin | 2146 | Cell Signaling |
| Myc-Tag | 2276 | Cell Signaling |
| p-AKT (Thr308) | 2965 | Cell Signaling |
| p-AKT (Ser473) | 4060 | Cell Signaling |
| AKT | 9272 | Cell Signaling |
| p-CREB (Ser133) | 9196 | Cell Signaling |
| CREB | 9197 | Cell Signaling |
| PKA substrate (RRXS/T, 9624) | 9621 | Cell Signaling |
| LC3B | 2775 | Cell Signaling |
| HA-Tag | 26183 | Thermo Fisher |
| CRTC2 for WB | PA5-72994 | Thermo Fisher |
| Flag-Tag | F3165 | Sigma-Aldrich |
| CRTC2 for IP | sc-271912 | Santa Cruz |
| Normal mouse IgG-AC | sc-2343 | Santa Cruz |
| Protein A/G PLUS-Agarose | sc-2003 | Santa Cruz |
| Sam68 (7-1) for IP | sc-1238 | Santa Cruz |
| Ubiquitin | VU101 | LifeSensors |
| P62/SQSTM1 | NBP1-48320 | Novusbio |
| SREBP-2 | ab30682 | Abcam |
| LDLR | ab30532 | Abcam |
| HMGCS1 (D1Q9D) | 42201 | Cell Signaling |
| HMGCR | PA5-37367 | Thermo Fisher |
| PCSK9 | AF3985 | R&D Systems |
| MOMA2 | MCA519G | BioRad |
| Anti-mouse IgG, HRP-linked antibody | 7076 | Cell Signaling |
| Anti-rabbit IgG, HRP-linked antibody | 7074 | Cell Signaling |

GTT, PTT, GcTT, and ITT

Mice were fasted overnight (16 h, GTT and PTT) or for 6 h (GcTT and ITT), and then glucose, sodium pyruvate, glucagon, or insulin was administered via intraperitoneal injection. Doses were as follows: i) 2 g/kg body weight glucose, 2 g/kg sodium pyruvate, 10 μg/kg glucagon, and 1 U/kg insulin for studies in non-diabetic models; ii) 1 g/kg glucose, 1 g/kg sodium pyruvate, 10 μg/kg glucagon, and 2 U/kg insulin for studies in the HFD-STZ model; and iii) 0.5 g/kg glucose, 0.5 g/kg sodium pyruvate, 5 μg/kg glucagon, and 2 U/kg insulin for db/db mice. Blood glucose levels were measured from tail bleeds 0, 15, 30, 45, 60, 90, and 120 min after injection, as previously described⁵⁵.

Hyperinsulinemic-Euglycemic Clamping

For catheter implantation surgery, mice were anesthetized with 2% inhaled isoflurane, and analgesia was provided via buprenorphine (0.05 mg/kg) and carprofen (2.5 mg/kg) injection before surgery, after surgery, and daily for 3 days during the recovery period. Home-made catheters were filled with heparinized-glycerol locking solution, implanted under the back skin of mice, and threaded into the left carotid artery for blood sampling and the right jugular vein for infusion. Five to six days after surgery, the mice were fasted for 5 h, and the catheters were externalized under isoflurane anesthesia and connected to syringe pumps (CMA 402, Harvard Apparatus, Holliston, Mass.; NE-300, New Era Pump Systems, Farmingdale, N.Y.). Mice were awake, unhandled, and able to freely move in a plastic container during the study. The protocol consisted of a 120-min tracer equilibration period (from time, t=−120 to 0 min) beginning at 12:00 μm after a 4-hour fasting period. For assessment of basal glucose turnover, [3-³H]-Glucose (Perkin Elmer, Boston, Mass.) was delivered at t=−120 min first as a bolus dose (5 μCi), and then via continuous infusion (0.05 μCi/min) for 2 h. The insulin clamp was initiated at t=0 min via continuous infusion of human insulin (1.2 mU/kg per min; Humulin R; Eli Lilly, Indianapolis, Ind.) and maintained until t=120 min, and the [3-³H]-Glucose infusion was increased to 0.1 μCi/min to minimize the change in specific activity from the equilibration period. The specific activity varied by less than 10% from the average during the last 40 min of the clamping period, and the slope of specific activity over time did not differ significantly from the slope at t=0. Blood glucose levels were measured every 10 min with a Bayer Contour Blood Glucose Meter (Ascensia Diabetes Care US, Inc, Parsippany, N.J.), and euglycemia (100 mg/dL) was maintained by adjusting the rate of infusion for the 20% glucose solution. Blood samples (50 μL) were taken for assessments of glucose, insulin, and free fatty acid levels, and for glucose specific activity in plasma, at t=−5, 90, 100, 110, and 120 min; red blood cells were collected from the samples via centrifugation and injected via an arterial catheter to prevent hematocrit deficit. At the end of clamp experiment, the mice were sacrificed, and livers were snap frozen in liquid nitrogen for measurements of glycogen content. The insulin infusion rate was based on observations in pilot studies.

Biochemical Assays

Deproteinized plasma samples (20 μL) during clamping were used for measurements of total glucose concentrations by using Glucose Assay kit (Cell Biolabs, San Diego, Calif.) and for determination of [3-³H]-Glucose and ³H₂O as Applicants previously described⁵⁶. Radioactivity was measured with a Multi-purpose Scintillation Counter LS6500 (BECKMAN COULTER), and plasma glucose specific activity (SA) was calculated from the ratio of plasma glucose radioactivity (dpm) to plasma glucose content (mg) multiplied by the ratio of chemical standard evaporated (CSE) to chemical recovered standard (CRS); then, the [3-³H]-glucose infusion rate (GIR; dpm/kg per min) was calculated from the CSE, the glucose disposal rate (Rd; mg/kg per min) was calculated as the ratio of the GIR to the plasma glucose SA at the end of the basal period and during the final 30 min of clamping, and the hepatic glucose production rate (Endo Ra; mg/kg per min) was calculated by subtracting the steady-state GIR from Rd. The radioactivity of ³H in hepatic glycogen was determined by digesting tissue samples in KOH and precipitating glycogen with ethanol⁵⁷. Glycogen synthesis rate was converted by dividing hepatic tracer glycogen infusion rate (dpm/g liver) by plasma tracer glucose SA.

Measurement of Serum Insulin, Cholesterol, and Triglyceride

Serum insulin was measured using the Ultra Sensitivity Mouse Insulin ELISA Kit (Crystal Chem, #90080) according to manufacturer's protocol. Total cholesterol and triglyceride levels were measured using Infinity Cholesterol Reagent (TR13421) and Infinity Triglycerides Reagents (TR22421).

HFD-Induced Diabetes Model

Four week-old Sam68^{LKO} and Sam68^{f/f} mice were fed a HFD (58% of energy from fat; D12331, Research Diet) for 3 months, fasted for 6 h, intraperitoneally injected with a single dose of streptozotocin (STZ) (100 mg/kg body weight; freshly dissolved in 0.1 M sodium citrate pH 4.5), and then maintained on the HFD for one more month. Blood glucose measurements were performed at day 5 after STZ injection and at the end of the HFD feeding protocol, and mice that were hyperglycemic (3 h fasting blood glucose levels ≥250 mg/dL) at both time points were diagnosed with diabetes (i.e. insulin defective stage of T2D)⁵⁸ and used in subsequent experiments.

Human Liver Samples

Studies with human tissues were approved by the Institutional Review Board (IRB) for Human Use of the University of Alabama at Birmingham (Protocol #: IRB-300002079) and performed in compliance with the Belmont Report and Declaration of Helsinki. Liver samples were obtained via needle biopsy from patients with or without diabetes during bariatric surgery at UAB. Informed consent was obtained from all subjects, and patient characteristics are listed in Table 3.

TABLE 3
Demographic information of diabetic and
non-diabetic patients

| Age | | | | |
|---|---|---|---|---|
| (years) | Sex | Ethnicity | Diabetes-Liver Disease | Medications |
| 48 | Female | White | Yes-Hepatic adenoma (Benign) | Metformin |
| 36 | Female | White | Yes-Hepatic adenoma (Benign) | Metformin |
| 56 | Female | White | Yes-Hemangioma (Benign) | Metformin |
| 78 | Female | White | Yes-Normal | Metformin |
| 68 | Female | White | Yes-Uninvolved | Metformin |
| 74 | Female | Asian | Yes-Uninvolved | Metformin |
| 54 | Male | White | Yes-Uninvolved | Insulin |
| 58 | Male | White | Yes-Uninvolved | Insulin |
| 73 | Male | White | Yes-Uninvolved | Insulin |
| 71 | Male | Black | Yes-Normal | Metformin |
| 49 | Female | White | No-Hemangioma (Benign) | N/A |
| 41 | Female | White | No-Hepatic adenoma (Benign) | N/A |
| 44 | Female | White | No-Hepatic adenoma (Benign) | N/A |
| 31 | Female | White | No-Hepatic adenoma (Benign) | N/A |
| 46 | Female | Black | No-Hepatic adenoma (Benign) | N/A |
| 32 | Female | White | No-Hepatic adenoma (Benign) | N/A |
| 52 | Female | White | No-Hepatic adenoma (Benign) | N/A |
| 42 | Female | Black | No-Hepatic adenoma (Benign) | N/A |
| 62 | Female | White | No-Normal | N/A |
| 50 | Female | White | No-Normal | N/A |

Plasmid Construction and Transfection

The pcDNA3-Flag-CRTC2 and pCMV6-COP1-Myc-Flag plasmids were purchased from Addend and OriGene Technologies, respectively. The full-length pcDNA3-HA-Sam68 vector and serial truncation vectors (pcDNA3-HA-Sam68^{ΔN-Ter(1-157aa)}, pcDNA3-HA-Sam68^{ΔCK(257-259aa)}, pcDNA3-HA-Sam68^{ΔP3-P4(280-346aa)}, and pcDNA3-HA-Sam68^{ΔC-Ter(347-443aa)}) were constructed by GenScript (Piscataway, N.J.) and verified by sequencing. The pcDNA3-Flag-CRTC2^{K628R} plasmid was a gift from Dr. Marc Montminy (Salk Institute for Biological Studies)⁵⁹. Plasmids were transfected into 293T or HepG2 cells by using Lipofectamine 3000 Transfection Reagent (Invitrogen, Inc.).

Adenoviral Vectors and Adeno-Associated Viral Vectors

Adenoviral vector Ad-CRTC2^{K628R} was a gift from Dr. Marc Montminy (Salk Institute for Biological Studies)⁵⁹, and Ad-Sam68 and Ad-GFP were generated by Vector Biolabs (U Penn, Malvern, Pa.). The virus was administered to mice by tail vein injection at 2.0×10⁹ infection units (IFU) per mouse and was applied to cultured primary hepatocytes at a dose of 5×10⁶ PFU (plaque-forming unit) per 1.0×10⁶ cells, respectively. AAV vectors (AAV8-TBG-eGFP, AAV8-TBG-iCre, AAV8-GFP-murineSam68-shRNA, and AAV8-GFP-Scrmb-shRNA) were generated by Vector Biolabs (U Penn, Malvern, Pa.) and administered by tail vein injection at 5×10¹¹ genome copies (AAV8-TBG-eGFP or AAV8-TBG-iCre) or 1×10¹² genome copies (AAV8-GFP-murinSam68-shRNA or AAV8-GFP-Scrmb-shRNA) per mouse; transgene expression was evaluated 3 weeks after administration.

Hepatocyte Isolation

Primary hepatocytes were isolated via a two-step perfusion procedure with liver perfusion media (Krebs-Ringer Biocarbonate Buffer, Sigma, K4002) and liver digest buffer (Krebs-Ringer Biocarbonate Buffer with 0.1-0.15% collagenase) as previously described⁶⁰. After isolation, cells were cultured on collagen-coated plates in DMEM containing 4.5 g/liter glucose and supplemented with 10% FBS, and 1% penicillin and streptomycin. After 6 h of attachment, the medium was replaced, and the cells were incubated overnight before use in subsequent experiments.

Western Blotting

For protein extraction, 1×10⁷ cells or 100 mg of frozen tissue were homogenized in 1 mL RIPA lysis buffer (50 mM Tris-HCl pH 8.0, 1 mM EDTA, 1% Triton X-100, 0.1% SDS, 150 mM NaCl) that contains protease-inhibitor (Sigma, 4693132001) and phosphatase-inhibitor (Sigma, 4906837001) cocktail. Samples were incubated with agitation for 30 min at 4° C. and centrifuged at 13000 rpm for 10 min at 4° C.; then, the protein concentration in the supernatant was determined via bicinchoninic acid (BCA) assay (Pierce). For immunoblotting, proteins in the supernatant were denatured by heating at 95° C. for 10 min, separated by SDS-PAGE, and then transferred onto a polyvinylidene difluoride (PVDF) membrane (Bio-Rad). The membrane was incubated in 5% non-fat milk blocking buffer (Tris-buffered saline [TBS]) for 1 h, incubated with primary antibody in TBS-containing 3% bovine serum albumin (BSA) overnight at 4° C., washed 3 times with TBS (0.5% Tween 20), incubated with secondary antibody, washed with TBS Tween 20, and then developed with Enhanced Chemiluminescence Detection Reagents (ECL, Thermo Fisher). Protein signals were imaged with a Bio-Rad ChemiDoc System. Antibodies are listed in Table 2.

Co-Immunoprecipitations (Co-IP)

Immunoprecipitation (IP) was performed as previously described²⁵. Briefly, cells were lysed in 1% NP40 buffer (50 mM Tris-HCl pH 8.0, 1% Triton X-100, 150 mM NaCl, 0.25% sodium deoxy cholate and protease inhibitor cocktail), and samples were incubated with protein A/G plus agarose-conjugated antibody (Santa Cruz) overnight at 4° C. and washed; then, the immunoprecipitates were eluted by boiling for 10 min, and extracts were analyzed by Western Blotting. To evaluate CRTC2 ubiquitination, IP Lysis Buffer was supplemented with a complete protease inhibitor cocktail (Thermo Fisher, 4311235), a deubiquitin enzyme inhibitor PR-619 (30 uM, EMD Millipore), and 1,10-Phenanthroline (5 mM, EMD Millipore).

Quantitative Real-Time Polymerase Chain Reaction (q RT-PCR)

Total RNA was isolated with TRIzol Reagent and reverse transcribed into cDNA with Reverse Transcription Reagents (Applied Biosystems); then, tissue mRNA levels were determined by qPCR (AB13000; Applied Biosystems) with SYBR Green Real-Time PCR Master Mix (Applied Biosystems). Duplicate reactions were performed for each sample, and the relative mRNA expression level for each gene was calculated via the 2(−ΔΔCt) method and normalized to β-actin, which was arbitrarily set to 1. Primers are listed in Table 1.

Chromatin Immunoprecipitation (ChIP)

ChIP experiments were performed using a ChIP assay kit (EMD Millipore) as directed by the manufacturer's instructions. Briefly, cells were fixed in 1% formaldehyde for 10 min at room temperature; then, the cells were quenched by adding glycine to a final concentration of 125 mM, washed twice with PBS containing a protease inhibitor cocktail (1 mM PMSF, 1 μg/ml aprotinin, and 1 μg/ml pepstatin A), pelleted, resuspended in 200 μL ChIP lysis buffer (1% SDS, 10 mM EDTA, and 50 mM Tris, pH 8.1), and sonicated in a ultrasonic processor (Sonicator 3000; Misonix) to shear the DNA into ˜500-bp segments. Samples were precleared with protein A agarose beads and then immunoprecipitated with CRTC2 antibody (sc-271912; Santa Cruz Biotechnology, Inc.) or rabbit IgG at 4° C. overnight. Immunoprecipitated DNA complexes were eluted from the agarose beads twice by adding elution buffer (1% SDS, 0.1 M NaHCO₃, pH 8.0) at room temperature for 15 min, and cross-linking was reverse by heating at 65° C. for 2 h. The immunoprecipitated DNA was analyzed by quantitative real-time PCR with primers for regions encompassing the CRE motif in the promoters of PGC-1α, PEPCK, and G6Pase. Primers are listed in Table 1.

Hepatic Glucose Production

Primary hepatocytes were seeded into six-well plates (1×10⁶ cells per well), cultured overnight in DMEM with 10% FBS, washed three times with PBS, and then incubated for 4 h in glucose production buffer (consisting of glucose-free DMEM pH 7.4 without phenol red and supplemented with 20 mM sodium lactate, 2 mM sodium pyruvate, and 1 mM glycerol) containing 100 nM glucagon, 10 μM forskolin, or 100 μM Bt2-cAMP; then, 0.5 mL medium was collected, and the glucose concentration was measured with a colorimetric glucose assay kit (Eton Bioscience, Inc). Readings were normalized to the total protein content in whole-cell lysates.

Cytoplasmic and Nuclear Fractionation

Cytoplasmic and nuclear fractions were isolated with a commercially available kit (78835; Thermo Fisher Scientific) as directed by the manufacturer's instructions. For assessments of glucagon-induced nuclear translocation in the liver, mice were fasted for 4 h and intraperitoneally injected with glucagon (30 μg/kg body weight) via the inferior cava; 10 min later, the liver was collected, and cytoplasmic and nuclear proteins were isolated.

Analysis of In Vivo Insulin Signaling

Mice were fasted for 16 h and intraperitoneally injected with insulin (1 U/kg) or saline. Twenty minutes later, mice were euthanized, and the liver, muscle and fat tissues were quickly excised, snap frozen in liquid nitrogen, and stored at −80° C. until use. For evaluation of insulin signaling, tissues were homogenized in RIPA buffer containing a protease- and phosphatase-inhibitor cocktail (Sigma). Tissue proteins were extracted and evaluated via Western blotting with primary antibodies against Ser-473-AKT, T308-AKT and total AKT (Table 2) as previously described³¹

PKA Activity Assay

Cells were starved in FBS-free DMEM for 4 h, treated with 100 nM glucagon, 10 μM forskolin, or 100 μM Bt2-cAMP for 30 min, and then lysed in cell lysis buffer (Tris-based, pH 8 buffer with 1% NP-40) containing protease-inhibitors and phosphatase-inhibitors cocktail (Sigma). PKA activity was determined with a PKA Colorimetric Activity Kit (Invitrogen, #EIAPKA) as directed by the manufacture's protocol.

Targeting Protein Domain Prediction by Text Pattern Search and Complementary Hydropathy

Mouse Sam68 and CRTC2 sequences were obtained from UniProt (accession Q60749-1, and accession Q3U182-1, respectively). Combined text pattern search method⁶¹ and the hydropathic analysis method⁶² were used to predict potential Sam68 C-terminal domain (P5, “YY” and NLS region) binding sites in CRTC2. The sequences of proline-rich region of P5 (356-363 aa), the tyrosine-rich “YY” region (366-411 aa) and nuclear localization signal (420-443 aa) region in C-terminal of Sam68 were separately converted into binary (+ or −) hydrophobicity maps based on the sign of each amino acid's hydrophobicity by the Kyte and Doolittle scale. Hydrophobicity map for each potential binding motif in Sam68 in both forward and reverse orientations was scanned across a hydrophobicity map for the whole protein sequence of CRTC2. The percentage of match was calculated by dividing complementary (+/−) pairs to all matched pairs. The degree of complementary hydropathy was calculated (see Equation 1⁶²) based on the Kyte Doolittle hydropathy index⁶³.

$$C = \frac{{\sum }_{i = 1}^{L} \vert H ( i ) - {H}^{′} ( i ) \vert}{L * 9}$$

In Equation 1, C is the degree of complementary hydropathy, H(i) and H′(i) are the hydropathy indices of the amino acids in the motif and target sequences respectively at position i, and L is the length of the protein fragment. The degree of complementary hydropathy can range from 0 to 1.0. Only ≥75% percentage of match and 0.42 degree of complementary hydropathy were considered as high possibility for protein interaction in this study. The hydropathy plot was generated according to hydropathic score of each amino acid.

En Face Quantification of Atherosclerotic Lesions in the Aorta

Mice were anesthetized and perfused with 10 mL PBS via the left ventricle. The full-length aorta was carefully excised and fixed in 10% formalin for 3 days. After the surrounding adventitial fat tissue was removed under a dissection microscope, the aorta was then opened longitudinally and pinned on a black rubber plate. The aorta was treated with 60% isopropanol for 10 min, then stained with oil red 0 solution (3 mg/mL in 60% isopropanol) for 15 min and de-stained with 60% isopropanol for 5 min (to eliminate background staining). After the images were captured, the oil-red 0-stained atherosclerotic lesion area was quantitated using the NIH Image J soft-ware.

Immunofluorescence Staining of Macrophage Infiltration

Aortic roots were cut into cross-sections, and the sections were blocked with 5% BSA for 1 h and incubated overnight at 4° C. with MOMA2 antibody (1:100). After wash, the sections were incubated with the Alexa Fluor 488-conjugated goat anti-mouse IgG secondary antibody (1:200) for 1 h. Nuclei were counterstained with Hoechst 33342 (Blue, 1:1000) for 10 min. The macrophage infiltration was quantified by measuring the areas of the MOMA-2-stained regions, and positively stained areas were measured with NIH image J software.

Statistical Analysis

Data are presented as mean±SEM. Statistical significance between two groups was evaluated via the unpaired two-tailed Student's t test and among 3 or more groups via one-way or two-way analysis of variance (ANOVA) with one or two independent variables. A p-value of less than 0.05 was considered significant.

### Example 8—Ablation of Hepatic Sam68 Protects Against Hypercholesterolemia and Atherosclerosis

Perturbations in hepatocyte cholesterol metabolism can contribute to atherosclerosis, coronary artery disease, and stroke by increasing cholesterol levels in the blood (hypercholesterolemia), particularly low-density lipoprotein cholesterol (LDL-C)⁶⁴. Many common cholesterol-lowering drugs target the liver, including proprotein-convertase-subtilisin/kexin-type-9 (PCSK9) inhibitors, which promote the uptake of circulating LDL-C by stabilizing the LDL receptor, and statins, which suppress hepatic cholesterol biosynthesis⁶⁴. Notably, the transcription factor sterol regulatory element-binding protein 2 (SREBP-2) regulates both cholesterol uptake and biosynthesis by activating the expression of PCSK9 and of two enzymes that catalyze the rate-limiting steps of cholesterol production-3-hydroxy-3-methyl-glutaryl coenzyme A reductase (HMGCR) and squalene monooxygenase (SQLE)⁶⁴. Thus, strategies targeting SREBP-2 have been proposed both as potential treatments for atherosclerosis and for screening novel cholesterol-lowering drugs^{65,66}.

Src-associated-in-mitosis-of-68 kDa (Sam68; also known as KH-domain-containing RNA-binding signal-transduction-associated 1 [KHDRBS1]) is a member of the signal-transducer-and-activator-of-RNA (STAR) family of RNA-binding proteins and functions as an adaptor protein during RNA processing. Sam68 is known to participate in numerous cellular processes (e.g., gene transcription and kinase- and growth-factor-signaling), as well as adipogenic differentiation³⁰, and Applicants have shown that global Sam68 deletions impede the inflammatory response to vascular injury in mice⁵¹. The experiments reported here indicate that Sam68 may also have a key role in cholesterol metabolism and atherosclerosis.

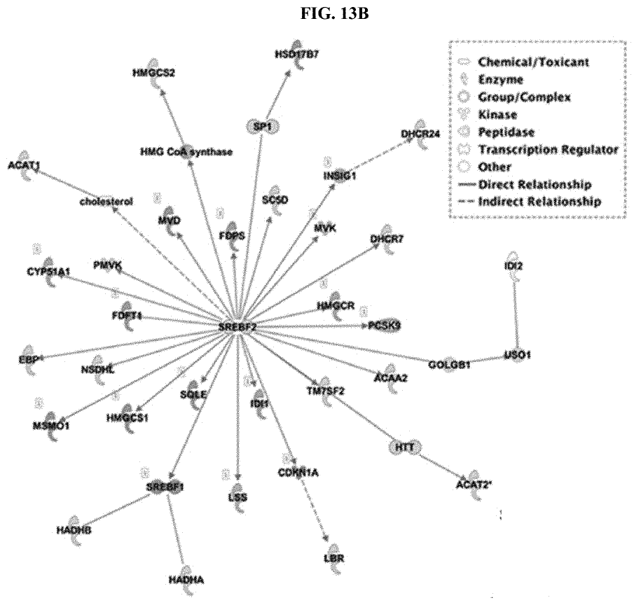

Sam68-floxed (Sam68^{f}1 mice were generated via homologous recombination and then serially crossed with Alb-cre mice to produce mice carrying a hepatocyte-specific Sam68 knockout mutation (Alb-cre;Sam68^{f/f} or Sam68^{LKO} mice) (FIG. 13A). When mRNA sequencing analyses were conducted in the livers of Sam68^{LKO} mice and their wildtype (Sam68^{f/f}) littermates, the differentially expressed genes (DEGs) were predominantly involved in cholesterol metabolism and included nearly half of the enzymes that participate in cholesterol biosynthesis (FIG. 13A). Furthermore, the expression of SREBP-2 and of SREBP-2-targeted genes that have a role in cholesterol metabolism was significantly lower (activation z-score=−2.673, p≤0.001) in Sam68^{LKO} mice than in Sam68^{f/f} mice (FIG. 13B). Thus, Applicants investigated whether the loss of Sam68 expression in liver cells could protect mice from hypercholesterolemia and atherosclerosis.

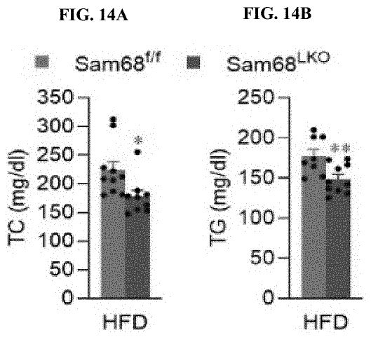

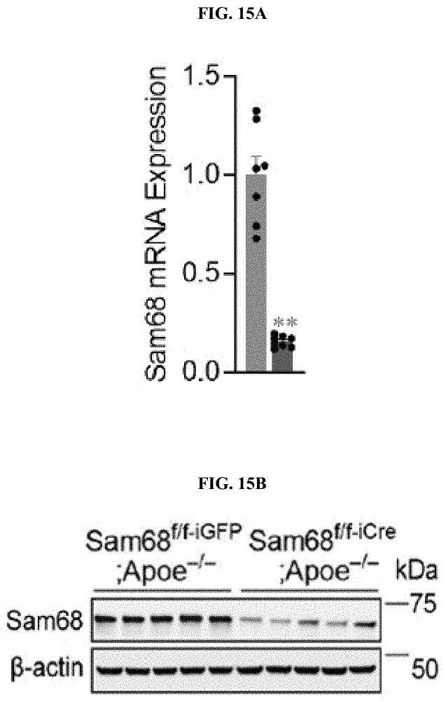

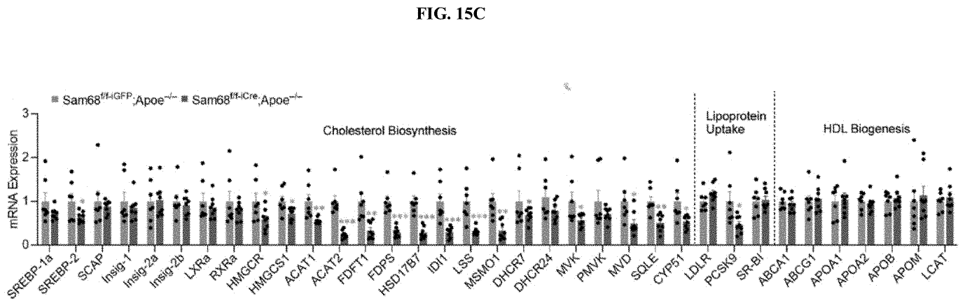

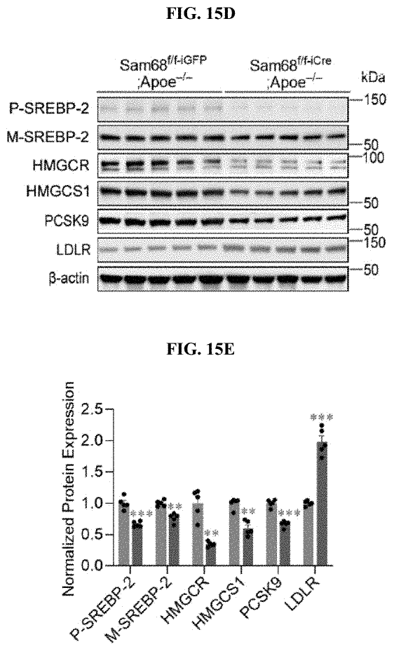

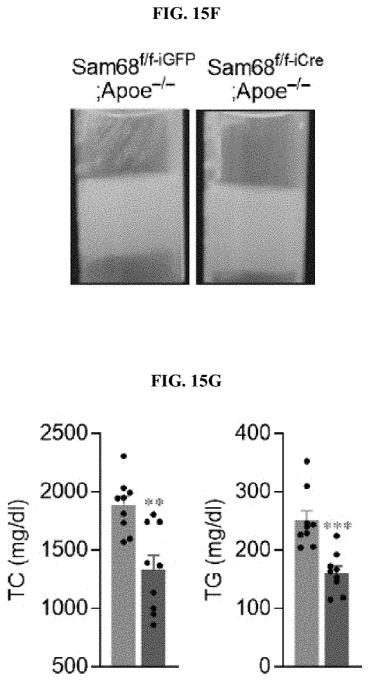

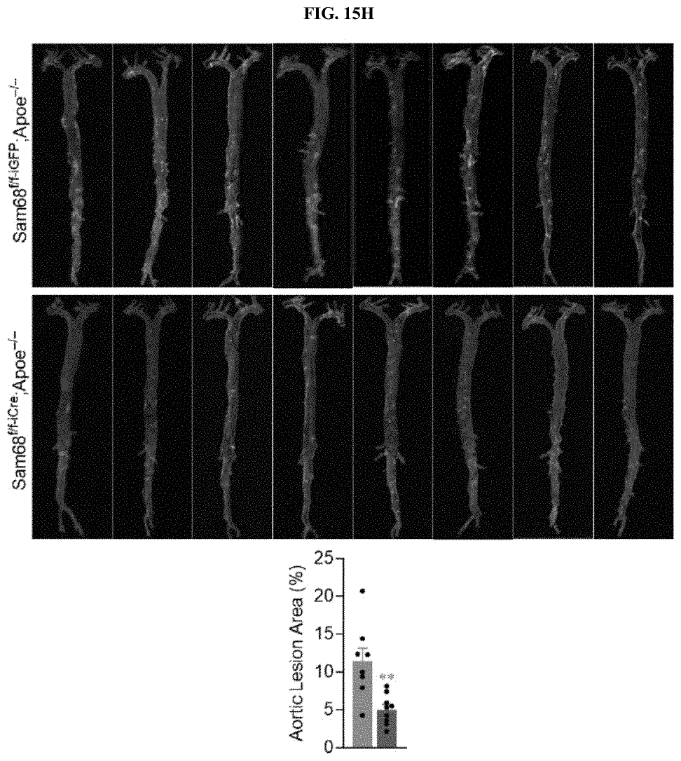

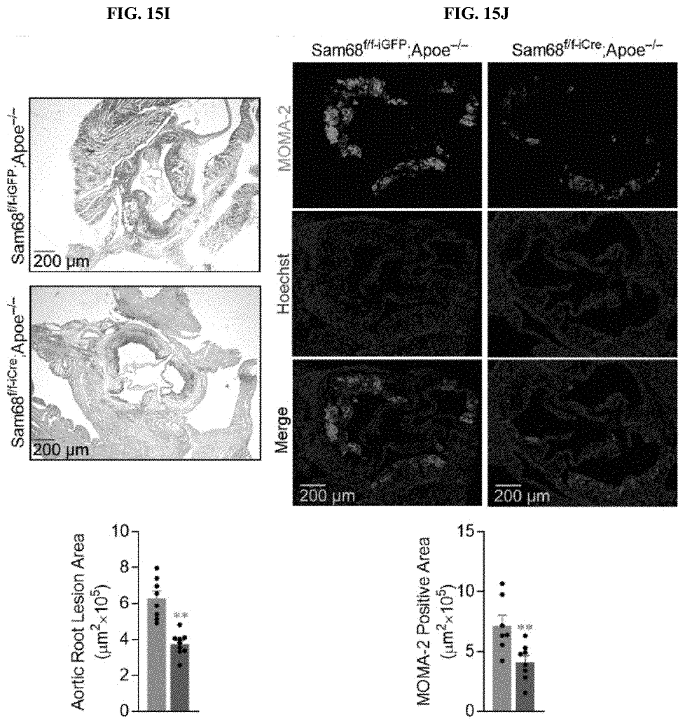

Experiments were conducted in Sam68^{f/f};Apoe^{-/-} mice. Sam68 was deleted in liver cells by intravenously injecting the animals with adeno-associated virus 8 (AAV8) coding for the expression of Cre recombinase driven by the hepatocyte-specific thyroxine-binding globulin (TBG) promoter (Sam68^{f/f-iCre};Apoe^{-/-} mice). Control assessments were conducted in mice administered AAV8 coding for TBG-regulated GFP expression (Sam68^{f/f-iGFP};Apoe^{-/-} mice), and subsets of animals in both groups were sacrificed 3 weeks after AAV8 administration to confirm that Sam68 mRNA and protein levels were dramatically downregulated in the livers of Sam68^{f/f-iCre};Apoe^{-/-} mice (FIGS. 15A-15B). Hypercholesterolemia and atherosclerosis were induced in the remaining mice by feeding them a Western diet for 16 weeks; then, the animals were euthanized and evaluated for hepatic expression of cholesterol-metabolism genes (FIG. 15C-15E), serum lipid levels (FIG. 15F), and aortic atherosclerosis (FIG. 15H). mRNA levels of SREBP-2 and numerous SREBP-2-targeted cholesterol biosynthesis genes were significantly lower in Sam68^{f/f-iCre};Apoe^{-/-} mice than in Sam68^{f/f-iGFP};Apoe^{-/-} mice; PCSK9 mRNA expression also declined in response to hepatic Sam68-knockout, but mRNA levels for nine other genes that are involved in lipoprotein uptake or high-density lipoprotein (HDL) biogenesis were equivalent in the two groups (FIG. 15C). Protein levels of hepatic SREBP-2 precursor (P-SREBP-2), mature SREBP-2 (M-SREBP-2), HMGCR, 3-Hydroxy-3-Methylglutaryl-CoA Synthase 1 (HMGCS1), and PCSK9 were also significantly downregulated in Sam68^{f/f-iCre};Apoe^{-/-} mice, while the abundance of LDL receptor protein was markedly greater in Sam68^{f/f-iCre};Apoe^{-/-} mice than in Sam68^{f/f-iGFP};Apoe^{-/-} mice (FIG. 15D), and these changes in the expression of cholesterol metabolism genes were accompanied by significant declines in measurements of serum total cholesterol (TC) and triglycerides (TG) (by ˜30% and ˜36%, respectively) in Sam68^{f/f-iCre};Apoe^{-/-} mice (FIGS. 15F-15G). Applicants also evaluated the progression of atherosclerosis by staining aortic sections with Oil Red O to quantify lesion formation and with MOMA-2 antibodies to evaluate macrophage infiltration of the arterial wall: both en face and cross-sectional lesion areas, as well as regions of macrophage infiltration, were significantly smaller (by 56%, 40%, and 42%, respectively) in aortas from Sam68^{f/f-iCre};Apoe^{-/-} mice than in the aortas of Sam68^{f/f-iGFP};Apoe^{-/-} mice (FIGS. 15H-15J). Further, plasma TC (FIG. 14A) and TG (FIG. 14B) levels were measured in HFD-fed Sam68^{f/f} mice and Sam68^{LKO} mice after 16 h fasting and 6 h refeeding, which show that deletion of Hepatic Sam68 in mice mitigates HFD-induced hypercholesterolemia.

In conclusion, the data demonstrate that hepatic Sam68 expression potentiates the transcriptional output of SREBP-2, thereby facilitating the expression of genes that participate in cholesterol biosynthesis, as well as the progression of hypercholesterolemia and atherosclerosis in Apoe^{-/-} mice. Thus, strategies for targeting Sam68 expression in the liver may prove to be useful for the treatment of atherosclerosis.

### REFERENCES

1. Forbes, J. M. & Cooper, M. E. Mechanisms of diabetic complications. Physiol Rev 93, 137-188 (2013).
2. Rines, A. K., Sharabi, K., Tavares, C. D. & Puigserver, P. Targeting hepatic glucose metabolism in the treatment of type 2 diabetes. Nat Rev Drug Discov 15, 786-804 (2016).
3. Magnusson, I., Rothman, D. L., Katz, L. D., Shulman, R. G. & Shulman, G. I. Increased rate of gluconeogenesis in type II diabetes mellitus. A 13C nuclear magnetic resonance study. J Clin Invest 90, 1323-1327 (1992).
4. Hunter, R. W., et al. Metformin reduces liver glucose production by inhibition of fructose-1-6-bisphosphatase. Nat Med 24, 1395-1406 (2018).
5. Madiraju, A. K., et al. Metformin inhibits gluconeogenesis via a redox-dependent mechanism in vivo. Nat Med 24, 1384-1394 (2018).
6. Madiraju, A. K., et al. Metformin suppresses gluconeogenesis by inhibiting mitochondrial glycerophosphate dehydrogenase. Nature 510, 542-546 (2014).

Habegger, K. M., et al. The metabolic actions of glucagon revisited. Nat Rev Endocrinol 6, 689-697 (2010).
8. Dobbins, R. L., et al. Role of glucagon in countering hypoglycemia induced by insulin infusion in dogs. Am J Physiol 261, E773-781 (1991).
9. lourgenko, V., et al. Identification of a family of cAMP response element-binding protein coactivators by genome-scale functional analysis in mammalian cells. Proc Natl Acad Sci USA 100, 12147-12152 (2003).
10. Conkright, M. D., et al. TORCs: transducers of regulated CREB activity. Mol Cell 12, 413-423 (2003).
11. Luo, Q., et al. Mechanism of CREB recognition and coactivation by the CREB-regulated transcriptional coactivator CRTC2. Proc Natl Acad Sci USA 109, 20865-20870 (2012).
12. Dentin, R., Hedrick, S., Xie, J., Yates, J., 3rd & Montminy, M. Hepatic glucose sensing via the CREB coactivator CRTC2. Science 319, 1402-1405 (2008).
13. Herzig, S., et al. CREB regulates hepatic gluconeogenesis through the coactivator PGC-1. Nature 413, 179-183 (2001).
14. Wang, Y., et al. Inositol-1,4,5-trisphosphate receptor regulates hepatic gluconeogenesis in fasting and diabetes. Nature 485, 128-132 (2012).
15. Dentin, R., et al. Insulin modulates gluconeogenesis by inhibition of the coactivator TORC2. Nature 449, 366-369 (2007).
16. Rui, L. Energy metabolism in the liver. Compr Physiol 4, 177-197 (2014).
17. Miller, R. A., et al. Biguanides suppress hepatic glucagon signalling by decreasing production of cyclic AMP. Nature 494, 256-260 (2013).
18. Fumagalli, S., Totty, N. F., Hsuan, J. J. & Courtneidge, S. A. A target for Src in mitosis. Nature 368, 871-874 (1994).
19. Lukong, K. E. & Richard, S. Sam68, the KH domain-containing superSTAR. Biochim Biophys Acta 1653, 73-86 (2003).
20. Bielli, P., Busa, R., Paronetto, M. P. & Sette, C. The RNA-binding protein Sam68 is a multifunctional player in human cancer. Endocr Relat Cancer 18, R91-R102 (2011).
21. lijima, T., et al. SAM68 regulates neuronal activity-dependent alternative splicing of neurexin-1. Cell 147, 1601-1614 (2011).
22. Huot, M. E., Brown, C. M., Lamarche-Vane, N. & Richard, S. An adaptor role for cytoplasmic Sam68 in modulating Src activity during cell polarization. Mol Cell Biol 29, 1933-1943 (2009).
23. Ramakrishnan, P. & Baltimore, D. Sam68 is required for both NF-kappaB activation and apoptosis signaling by the TNF receptor. Mol Cell 43, 167-179 (2011).
24. Fu, K., et al. Sam68 modulates the promoter specificity of NF-kappaB and mediates expression of CD25 in activated T cells. Nat Commun 4, 1909 (2013).
25. Zhou, J., et al. Regulation of vascular contractility and blood pressure by the E2F2 transcription factor. Circulation 120, 1213-1221 (2009).
26. Bielli, P., et al. The transcription factor FBI-1 inhibits SAM68-mediated BCL-X alternative splicing and apoptosis. EMBO Rep 15, 419-427 (2014).
27. Sellier, C., et al. Sam68 sequestration and partial loss of function are associated with splicing alterations in FXTAS patients. EMBO J 29, 1248-1261 (2010).
28. Li, L. J., et al. Decreased expression of SAM68 in human testes with spermatogenic defects. Fertil Steril 102, 61-67 e63 (2014).
29. Paronetto, M. P., et al. Sam68 marks the transcriptionally active stages of spermatogenesis and modulates alternative splicing in male germ cells. Nucleic Acids Res 39, 4961-4974 (2011).
30. Huot, M. E., et al. The Sam68 STAR RNA-binding protein regulates mTOR alternative splicing during adipogenesis. Mol Cell 46, 187-199 (2012).
31. Zhou, J., et al. Inhibition of Sam68 triggers adipose tissue browning. J Endocrinol 225, 181-189 (2015).
32. Sette, C., Messina, V. & Paronetto, M. P. Sam68: a new STAR in the male fertility firmament. J Androl 31, 66-74 (2010).
33. Bell, P., et al. Evaluation of adeno-associated viral vectors for liver-directed gene transfer in dogs. Hum Gene Ther 22, 985-997 (2011).
34. Oh, K. J., Han, H. S., Kim, M. J. & Koo, S. H. Transcriptional regulators of hepatic gluconeogenesis. Arch Pharm Res 36, 189-200 (2013).
35. Kim, C., Cheng, C. Y., Saldanha, S. A. & Taylor, S. S. PKA-I holoenzyme structure reveals a mechanism for cAMP-dependent activation. Cell 130, 1032-1043 (2007).
36. Liu, Y., et al. A fasting inducible switch modulates gluconeogenesis via activator/coactivator exchange. Nature 456, 269-273 (2008).
37. Hogan, M. F., et al. Hepatic Insulin Resistance Following Chronic Activation of the CREB Coactivator CRTC2. J Biol Chem 290, 25997-26006 (2015).
38. Ryu, D., et al. TORC2 regulates hepatic insulin signaling via a mammalian phosphatidic acid phosphatase, LIPIN1. Cell Metab 9, 240-251 (2009).
39. Wang, Y., et al. Targeted disruption of the CREB coactivator Crtc2 increases insulin sensitivity. Proc Natl Acad Sci USA 107, 3087-3092 (2010).
40. Sanchez-Jimenez, F. & Sanchez-Margalet, V. Role of Sam68 in post-transcriptional gene regulation. Int J Mol Sci 14, 23402-23419 (2013).
41. Dikic, I. Proteasomal and Autophagic Degradation Systems. Annu Rev Biochem 86, 193-224 (2017).
42. Rowley, W. R., Bezold, C., Arikan, Y., Byrne, E. & Krohe, S. Diabetes 2030: Insights from Yesterday, Today, and Future Trends. Popul Health Manag 20, 6-12 (2017).
43. Sharabi, K., et al. Selective Chemical Inhibition of PGC-1alpha Gluconeogenic Activity Ameliorates Type 2 Diabetes. Cell 169, 148-160 e115 (2017).
44. Quteineh, L., et al. CRTC2 polymorphism as a risk factor for the incidence of metabolic syndrome in patients with solid organ transplantation. The pharmacogenomics journal 17, 69-75 (2017).
45. Keshavarz, P., et al. Single nucleotide polymorphisms in genes encoding LKB1 (STK11), TORC2 (CRTC2) and AMPK alpha2-subunit (PRKAA2) and risk of type 2 diabetes. Molecular genetics and metabolism 93, 200-209 (2008).
46. Han, H. S., Choi, B. H., Kim, J. S., Kang, G. & Koo, S. H. Hepatic Crtc2 controls whole body energy metabolism via a miR-34a-Fgf21 axis. Nat Commun 8, 1878 (2017).
47. Saberi, M., et al. Novel liver-specific TORC2 siRNA corrects hyperglycemia in rodent models of type 2 diabetes. Am J Physiol Endocrinol Metab 297, E1137-1146 (2009).
48. Dullea, R., et al. Study of CRTC2 pharmacology using antisense oligonuceotides. Nucleic Acid Ther 24, 127-138 (2014).
49. Quintana-Portillo, R., Canfran-Duque, A., Issad, T., Sanchez-Margalet, V. & Gonzalez-Yanes, C. Sam68 interacts with IRS1. Biochem Pharmacol 83, 78-87 (2012).
50. Sanchez-Margalet, V., Gonzalez-Yanes, C., Najib, S., Fernandez-Santos, J. M. & Martin-Lacave, I. The expression of Sam68, a protein involved in insulin signal transduction, is enhanced by insulin stimulation. Cell Mol Life Sci 60, 751-758 (2003).
51. Han, S., et al. Sam68 impedes the recovery of arterial injury by augmenting inflammatory response. J Mol Cell Cardiol 137, 82-92 (2019).
52. Hotamisligil, G. S. Inflammation and metabolic disorders. Nature 444, 860-867 (2006).
53. Sheng, L., et al. NF-kappaB-inducing kinase (NIK) promotes hyperglycemia and glucose intolerance in obesity by augmenting glucagon action. Nat Med 18, 943-949 (2012).
54. Richard, S., et al. Ablation of the Sam68 RNA binding protein protects mice from age-related bone loss. PLoS Genet 1, e74 (2005).
55. Qiao, A., Jin, X., Pang, J., Moskophidis, D. & Mivechi, N. F. The transcriptional regulator of the chaperone response HSF1 controls hepatic bioenergetics and protein homeostasis. J Cell Biol 216, 723-741 (2017).
56. Kim, T., et al. Hepatic Glucagon Receptor Signaling Enhances Insulin-Stimulated Glucose Disposal in Rodents. Diabetes 67, 2157-2166 (2018).
57. Kim, H. J., et al. Differential effects of interleukin-6 and -10 on skeletal muscle and liver insulin action in vivo. Diabetes 53, 1060-1067 (2004).
58. Zhang, Z., et al. Sulforaphane prevents the development of cardiomyopathy in type 2 diabetic mice probably by reversing oxidative stress-induced inhibition of LKB1/AMPK pathway. J Mol Cell Cardiol 77, 42-52 (2014).
59. Altarejos, J. Y. & Montminy, M. CREB and the CRTC co-activators: sensors for hormonal and metabolic signals. Nat Rev Mol Cell Biol 12, 141-151 (2011).
60. Qiao, A., et al. Mouse patatin-like phospholipase domain-containing 3 influences systemic lipid and glucose homeostasis. Hepatology 54, 509-521 (2011).
61. Friedl, J. E. F. Mastering Regular Expressions: Powerful Techniques for Perl and Other Tools (Oreilly & Associates Inc, 1997).
62. Jacchieri, S. G., Torquato, R. & Brentani, R. R. Structural study of binding of flagellin by Toll-like receptor 5. J Bacteriol 185, 4243-4247 (2003).
63. Kyte, J. & Doolittle, R. F. A simple method for displaying the hydropathic character of a protein. J Mol Biol 157, 105-132 (1982)
64. Goldstein J L, Brown M S. A century of cholesterol and coronaries: From plaques to genes to statins. Cell. 2015; 161:161-172.
65. Tang J J, Li J G, Qi W, Qiu VVVV, Li P S, Li B L, Song B L. Inhibition of srebp by a small molecule, betulin, improves hyperlipidemia and insulin resistance and reduces atherosclerotic plaques. Cell Metab. 2011; 13:44-56.
66. Yu H, Rimbert A, Palmer A E, Toyohara T, Xia Y, Xia F, Ferreira L M R, Chen Z, Chen T, Loaiza N, Horwitz N B, Kacergis M C, Zhao L, Consortium B, Soukas A A, Kuivenhoven J A, Kathiresan S, Cowan C A. Gpr146 deficiency protects against hypercholesterolemia and atherosclerosis. Cell. 2019; 179:1276-1288 e1214.

What is claimed:

1. A composition comprising an effective amount of at least one inhibitor of Sam68, or a pharmaceutically acceptable form thereof.

2. The composition of claim 1, wherein the at least one inhibitor is selected from the group consisting of: small molecule inhibitors, peptide inhibitors, antibodies, viral vectors expressing an inhibitor RNA, and inhibitory RNA.

3. The composition of claim 2, wherein the at least one inhibitor of Sam68 comprises inhibitor RNA selected from the group consisting of: shRNA, siRNA, microRNA, and antisense-RNA.

4. The composition of claim 3, wherein the at least one inhibitor RNA comprises a sequence that is specifically hybridisable to a target Sam68 nucleotide sequence.

5. The composition of claim 2, wherein the at least one inhibitor of Sam68 comprises an antibody configured to bind to a target selected from the group consisting of: the N-terminal domain of Sam68 and the C-terminal domain of Sam68.

6. The composition of claim 2, wherein the at least one inhibitor of Sam68 comprises an antibody configured to decrease the association of Sam68 with at least one protein.

7. The composition of claim 6, wherein the at least one protein comprises CRTC2.

8. A method for treating a Sam68-mediated condition in a subject, comprising administering to said subject a therapeutically effective amount of at least one inhibitor of Sam68 that inhibits an activity of Sam68, or a pharmaceutically acceptable form thereof.

9. The method of claim 8, wherein the at least one inhibitor is selected from the group consisting of: small molecule inhibitors, peptide inhibitors, antibodies, viral vectors expressing an inhibitor RNA, and inhibitory RNA.

10. The method of claim 8, wherein the at least one inhibitor of Sam68 comprises inhibitor RNA selected from the group consisting of: shRNA, siRNA, microRNA, and antisense-RNA.

11. The method of claim 10, wherein the at least one inhibitor RNA comprises a sequence that is specifically hybridisable to a target Sam68 nucleotide sequence.

12. The method of claim 8, wherein the at least one inhibitor comprises an antibody configured to bind to a target selected from the group consisting of: the N-terminal domain of Sam68 and the C-terminal domain of Sam68.

13. The method of claim 8, wherein the at least one inhibitor comprises an antibody configured to decrease the association of Sam68 with another protein.

14. The method of claim 8, wherein the Sam68-mediated condition is selected from the group consisting of: diabetes, type 2 diabetes, obesity, cardiovascular disease, kidney disease, hypercholesterolemia, atherosclerosis and hyperglycemia.

15. The method of claim 8, wherein the subject is human.

16. The method of claim 8, wherein the subject is diagnosed with at least one condition selected from the group consisting of: diabetes, type 2 diabetes, obesity, cardiovascular disease, kidney disease, hypercholesterolemia, atherosclerosis and hyperglycemia.

17. The method of claim 8, wherein the subject is suspected of having at least one condition selected from the group consisting of: diabetes, type 2 diabetes, obesity, cardiovascular disease, kidney disease, hypercholesterolemia, atherosclerosis and hyperglycemia.

18. The method of claim 8, wherein the administration of the at least one inhibitor or pharmaceutically acceptable form thereof comprises two or more doses.

19. The method of claim 8, wherein the Sam68-mediated condition is treated or prevented.

20. The method of claim 8, wherein the administration is parenteral, pulmonary, intra-nasal, oral-gastric, buccal, or intravenous.
